(19) United States
(12) Patent Application Publication
(10) Pub. No.: US 2021/0046128 A1
(43) Pub. Date: Feb. 18, 2021

(54) MICROCIN THAT AMPLIFIES SHIGA TOXIN PRODUCTION OF FOODBORNE PATHOGEN E. COLI

(71) Applicant: THE PENN STATE RESEARCH FOUNDATION, University Park, PA (US)

(72) Inventors: HILLARY FIGLER, UNIVERSITY PARK, PA (US); EDWARD G. DUDLEY, UNIVERSITY PARK, PA (US)

(21) Appl. No.: 16/947,509

(22) Filed: Aug. 4, 2020

Related U.S. Application Data
(60) Provisional application No. 62/882,678, filed on Aug. 5, 2019.

(51) Int. Cl. A61K 35/744; A61K 35/761; C12N 15/70; C07K 14/195

(52) U.S. Cl. CPC A61K 35/744; C07K 14/195; C12N 15/70; A61K 35/761

(57) ABSTRACT

Disclosed herein are novel microcins, that inhibit growth of other E. coli and Shigella strains. It is produced by non-pathogenic E. coli strain 0.1229 and is found to be encoded on a 12.8 kilobase plasmid in strain 0.1229. The plasmid was identified in two other strains and was identified bioinformatically in other strains and species. Particular embodiments of the invention include a microcin that causes death of the susceptible cells in at little as two hours, and that can be used for killing pathogenic E. coli in vitro on surfaces and materials of interest, and in vivo, and further can be used prophylactically and therapeutically.

### CROSS REFERECE TO RELATED APPLICATION

This application claims priority under 35 U.S.0 § 119 to Provisional Patent Application Ser. No. 62/882,678, filed Aug. 5, 2019 herein incorporated by reference in its entirety.

### GOVERNMENT SPONSORSHIP

This invention was made with government support under Grant No. AI130856 awarded by the National Institutes of Health, Grant No. 2014-38420-21822 awarded by the United States Department of Agriculture and under Hatch Act Project No. PEN04644 awarded by the United States Deptmenent of Agriculture. The Government has certain rights in the invention

### SEQUENCE LISTING

The instant application contains a Sequence Listing which has been submitted in ASCII format via EFS-Web and is hereby incorporated by reference in its entirety. Said ASCII copy, created on Jul. 21, 2020, is named 2020-08-04_FIGLER_P13001_US01_SEQUENCE_ST25 and is 77 kilobytes in size.

### TECHNICAL FIELD

Aspects of the invention relate generally to bacteria, and microcins and compositions and methods for controlling and/or killing pathogenic bacteria (e.g., enterohemorrhagic and/or enterotoxigenic strains of E. coli), comprising use of a novel microcin which is not toxic to cattle and thus can eliminate E. coli O157 therefrom.

### BACKGROUND

Many strains of E. coli are nonpathogenic, and many strains are present in the human gut. Enterohemorrhagic E. coli (EHEC), however is transferred to humans from cattle, through contaminated meat, vegetables, and water. EHEC has a low infectious dose, and then colonizes the gut. The main symptoms include diarrhea, bloody diarrhea, vomiting and severe complications such as hemolytic uremic syndrome can occur. Hemolytic uremic syndrome or HUS leads to kidney failure and sometimes death. An important serotype of EHEC is O157:H7, the causative agent of recent outbreaks including romaine lettuce.

EHEC is the most common group of Shiga toxin producing E. coli. E. coli producing Shiga toxin are associated with 34 suspected outbreaks, 350 potential illnesses and 115 hospitalizations in 2015. Thus, making it one of the top 3 etiological agents causing foodborne outbreaks in the United States. Shiga toxin is not normally produced by the bacteria. Only after DNA damage, and the induction of the SOS response and phage, are the phage and Shiga toxin genes transcribed, then released by the cell via lysis. While, E. coli O157:H7 has been researched extensively, there is no treatment for these infections and antibiotics can often make matters worse.

### BRIEF SUMMARY OF PREFERRED EMBODIMENTS

Applicants have identified a microcin, that inhibits growth of other E. coli and Shigella strains. It is produced by non-pathogenic E. coli strain 0.1229 and is found to be encoded on a 12.8 kilobase plasmid in strain 0.1229. The plasmid was identified in two other strains and was identified bioinformatically in other strains and species.

This molecule shown to induce the DNA damage response in E. coli and leads to cell death and for E. coli converting phage, it causes phage induction and release of Shiga toxin which inhibits protein synthesis of eukaryotic cells and is associated with bloody diarrhea and hemolytic uremic syndrome (renal failure) in patients.

E. coli 0.1229 encodes three plasmids and plasmid 0.1229_3 encodes the novel microcin. The plasmid 0.1229_3 includes four ORFs that are necessary for Stx2a amplification phenotype these regions have been identified as hp1 (SEQ ID NO: 3), abc (SEQ ID NO: 5), cupin (SEQ ID NO: 6), and hp2 (SEQ ID NO: 10). In certain embodiments the microcin sequences and encoded proteins include one or more modifications so that the sequences do not read on naturally occurring sequences. Particular embodiments of the invention include a microcin that causes death of the susceptible cells in at little as two hours, and that can be used for killing pathogenic E. coli in vitro on surfaces and materials of interest, and in vivo, and further can be used prophylactically and therapeutically.

Additional embodiments of the invention identify the microcin present in E. coli plasmid 0.1229_3, includes ORFs encoding proteins synthesis, immunity, and export.

According to further embodiments of the invention, the novel microcin, designated herein as 0.1229_3 containing microcin, is utilized in a number of different and beneficial applications. In some instances, the use of 0.1229_3 containing microcin and/or bacteria that produce 0.1229_3 containing microcin advantageously replaces the use of antibiotics. According to yet further embodiments of the invention, the ability to inhibit a diversity of E. coli strains indicates that this microcin has utility to influence gut community composition and substantial utility for control of important enteric pathogens.

While multiple embodiments are disclosed, still other embodiments of the inventions will become apparent to those skilled in the art from the following detailed description, which shows and describes illustrative embodiments of the invention. Accordingly, the figures and detailed description are to be regarded as illustrative in nature and not restrictive.

### DECRIPTION OF THE FIGURES

The application file contains at least one drawing executed in color. Copies of this patent application publication with color drawings will be provided by the Office upon request and payment of the necessary fee.

FIG. 1 shows Stx2a levels of PA2 grown with various E. coli strains. Stx2a levels measured using the R-ELISA. LB refers to PA2 grown in monoculture. One-way ANOVA was used, and bars marked with an asterisk were significantly higher than LB (Dunnett's test, p<0.05).

FIG. 2A and FIG. 2B show the Stx2a levels and fluorescence of non-pathogenic E. coli, after PA2 growth in cell-free supernatant or W3110ΔtolC PrecA-gfp co-culture, respectively. Samples were normalized to cell density, OD₆₀₀ or OD₆₂₀, for Stx2a or fluorescence, respectively. One-way ANOVA was used, and levels marked with an asterisk were significantly higher than LB (Dunnett's test, p<0.05). FIG. 2C shows the Stx2a levels of PA2 grown in 0.1229 cell-free supernatant (left) or LB (right) with or without heat and Proteinase K treatments. Two-way ANOVA was used, and bars marked with an asterisk were significantly lower than untreated 0.1229 or LB (Dunnett's test, p<0.05).

FIGS. 3A-C show plasmid comparisons of individual 0.1229 plasmids and publicly available plasmids from NCBI by BLAST and visualized with BRIG. The colored rings indicate sequence similarity and the outer grey ring denotes annotated ORFs. The MccB17 operon is labeled mcbA-mcbG, and five antimicrobial resistance genes, macrolide (mph(A)), tetracycline (tet(A)), sulphonamide (sul1), aminoglycoside (aadA2), and trimethoprim (dfrA12) were identified. Ampicillin resistance gene (blaTEM-1B) and four ORFs, hp1, abc, cupin and hp2 are also labeled. NCBI accession numbers are pUTI89 (CP000244), pRS218 (CP007150), pECO-fce (CP015160), pSF-173-1 (CP012632), pHUSEC41-3 (NC_018997), and pEC16II (KU932034).

FIG. 4A and FIG. 4B show the Stx2a levels and fluorescence of 0.1229, its MccB17 knockouts and ZK1526, after PA2 growth in cell-free supernatant or W3110ΔtolC PrecA-gfp co-culture, respectively. Samples were normalized to cell density, OD₆₀₀ or OD₆₂₀, for Stx2a or fluorescence, respectively. One-way ANOVA was used, and bars marked with an asterisk were significantly lower than 0.1229 (Dunnett's test, p<0.05).

FIG. 5 shows Stx2a levels of PA2 grown in the cell-free supernatant of 0.1229, C600 containing p0.1229_3 and C600. Stx2a levels were measured using the R-ELISA. LB refers to PA2 grown in LB broth. One-way ANOVA was used, and bars marked with an asterisk were significantly higher than LB (Fisher's LSD test, p<0.05).

FIG. 6A shows Stx2a levels of PA2 grown in the cell-free supernatant of 0.1229 knockouts. FIG. 6B depicts the portion of p0.1229_3 with predicted open reading frames (ORFs). The colored triangles indicate the name of the regional knockout. FIG. 6C shows Stx2a levels of PA2 grown in the cell-free supernatant of 0.1229 knockouts. FIG. 6D shows Stx2a levels of PA2 grown in the supernatant of a C600 strain containing a portion of p0.1229_3 (pBR322::p0.1229_3²⁷⁴⁵⁻⁷⁹⁵). Stx2a levels measured using the R-ELISA. LB refers to PA2 grown in LB broth. One-way ANOVA was used, and bars marked with an asterisk were significantly lower than 0.1229 (Fisher's LSD test, p<0.05).

FIG. 7 shows fluorescence of W3110ΔtolC PrecA-gfp grown in co-culture with 0.1229, 0.1229ΔtolC and 0.1229ΔtolC pBAD18 containing strains. Samples were normalized to cell density, OD₆₂₀. One-way ANOVA was used, and bars marked with an asterisk were significantly higher than LB (Dunnett's test, p<0.05).

FIG. 8 shows fluorescence of plasmid expressing PrecA-gfp electroporated into MG1655, MG1655ΔtonB, MG1655ΔtonB pBAD24 and MG1655ΔtonB pBAD24::tonB. These strains were grown in co-culture with 0.1229, or by themselves (LB). Two-way ANOVA was used, and bars marked with an asterisk were significantly higher than their respective monoculture control (Dunnett's test, p<0.05).

FIG. 9A shows Stx2a levels of PA2 grown in the cell-free supernatant of 0.1229 and three human fecal E. coli isolates. Stx2a levels measured using the R-ELISA. LB refers to PA2 grown in LB broth. One-way ANOVA was used and bars marked with an asterisk were significantly higher than LB (Dunnett's test, p<0.05). FIG. 9B shows p0.1229_3 compared to the contigs of 99.0750, 91.0593 and 90.2723 using BLAST and visualized using BRIG.

FIG. 10A shows fluorescence of W3110ΔtolC PrecA-gfp grown with 0.1229 and 0.1229 regional knockouts, or alone indicated by LB. FIG. 10B shows fluorescence of W3110ΔtolC PrecA-gfp grown with 0.1229 and 0.1229 individual ORF knockouts, or alone indicated by LB. One-way ANOVA was used, and levels marked with an asterisk were significantly lower than 0.1229 (Dunnett's test, p<0.05).

FIG. 11 depicts a portion of p0.1229_3 compared to K. pneumoniae TR152 (SAMEA3729690), S. sonnei 143778 (SAMEA2057991), E. coli HUSEC41 (PRJEA73977), E. coli HVH206 (SAMN01885845). * indicates one amino acid (aa) difference in that ORF. # indicates seven aa differences in that ORF. The grey shaded region is >99.6% nucleotide identical between all strains.

FIG. 12 shows the Hp1 protein compared to genomes on Integrated Microbial Genomes & Microbiomes of DOE's Joint Genome Institute. Using BLASTp, isolates were compared, sorted by BIT score, and then one strain from the top ten species were selected. % identity ranged from 32 to 68%. Hp1 homologs are colored in red. 8/10 have ABC transporters adjacent to Hp1, colored in light blue or maroon. 9/10 have an annotated region similar to Cupin, colored in pale yellow, typically adjacent to the ABC transporter. DUF2164 is found in five strains, one in the reverse direction than Hp1. The Burkholderia cepacia strain encodes L-arabinose system upstream of Hp 1. The bracket indicates groupings of Hp1, ABC and Cupin.

FIG. 13 shows florescence of W3110ΔtolC PrecA-gfp grown with 0.1229 or 101 human fecal isolates from the E. coli Reference Center at Penn State. As a control, W3110 PrecA-gfp was grown by itself indicated by LB. One-way ANOVA was used, and levels marked with an asterisk were significantly higher than LB (Dunnett's test, p<0.05).

### DETAILED DESCRIPTION

While most E. coli strains are harmless, some serotypes can cause serious and even deadly diseases in a host, either as the result of exposure to the pathogenic bacteria via direct transmission from another infected host or by ingestion of or exposure to (e.g. handling) contaminated food products or from other sources of the bacteria (e.g. fomites). The methods and compositions are effective for killing (e.g. lysing) or preventing or decreasing the adverse effects of pathogenic Shigella sp. Those of skill in the art will recognize that phylogenetic studies indicate that Shigella is more appropriately treated as a subgenus of Escherichia, and that certain strains generally considered E. coli (e.g. E. coli 0157:H7) could be classified as Shigella. Herein, the phrases “pathogenic bacteria” and “pathogenic E. coli” encompasses both pathogenic E. coli and pathogenic Shigella, although the two may be discussed separately, for clarity and to accord with historic designations.

The term “pathogenic” refers to the ability of the bacterium to cause disease symptoms in one or more hosts. The targeted bacterium need not cause disease in all hosts that is it capable of colonizing. Successful colonization of some hosts by the bacterium may be entirely benign (asymptomatic, harmless, etc.). However, such non-susceptible hosts may serve as reservoirs of the pathogenic bacteria which, when transmitted to a susceptible host, cause disease. Herein, these two genera of hosts may be referred to as “disease susceptible hosts” and “non-disease susceptible hosts”, respectively, or simply as “susceptible hosts” and “non-susceptible hosts”. It will be understood that the methods of treatment described herein may be advantageously applied to both susceptible and non-susceptible hosts. For the susceptible hosts, treatment may prevent, cure (fully or partially) or ameliorate disease symptoms or prevent or decrease adverse effects that would otherwise be caused by pathogenic bacteria. These beneficial effects are brought about by killing and/or damaging established pathogenic bacteria, or by preventing, slowing or minimizing the growth of pathogenic bacteria to which the host is newly exposed. For non-susceptible hosts, treatment may destroy or lessen the number of pathogenic bacteria that can colonize the host or that might otherwise colonize the host, but for intervention using the methods and compositions described herein, thereby lessening or eliminating transmission of the pathogenic bacteria to other disease susceptible and non-susceptible hosts.

Susceptible hosts that may be subject to diseases caused by pathogenic E. coli are usually endotherms and may be mammals. Such mammals include but are not limited to primates (e.g. humans), livestock e.g. cattle, pigs, sheep goats, etc., especially neonates, juveniles, elderly or immune compromised individuals; etc. Alternatively, various avian species may also be subject to such infections, including but not limited to chickens, turkeys, ducks, etc. Non-susceptible hosts that may act as reservoirs of pathogenic bacteria that are passed to susceptible hosts include substantially the same endotherms described above as susceptible hosts.

Particular combinations of susceptible hosts and pathogenic bacteria include the following exemplary animal pathogens of interest: Poultry—avian pathogenic E. coli (APEC), Calves—E. coli K99 (which causes calf diarrhea), Swine—E. coli K88 (which causes post-weaning diarrhea).

For food safety: E. coli 0157:H7 The United States Department of Agriculture (USDA) “Big 6” STEC E. coli pathogens: E. coli serovars O26, O45, O103, O111, O121 and O145.

Diarrhoeagenic E. coli human pathovars: various enteropathogenic E. coli (EPEC) various enterohaemorrhagic E. coli (EHEC) various enterotoxigenic E. coli (ETEC) various enteroinvasive E. coli (EIEC; including Shigella) various enteroaggregative E. coli (EAEC) various so-called diffusely adherent E. coli (DAEC).

Extraintestinal E. coli (ExPEC) human pathovars: uropathogenic E. coli (UPEC) neonatal meningitis E. coli (NMEC).

Exemplary pathogenic Shigella species of interest which may be killed by the compositions and methods of the invention include but are not limited to: Serogroup A: S. dysenteriae, Serogroup B: S. flexneri, and Serogroup D: S. sonnei, and serotypes and serovars thereof.

In addition, contamination with pathogenic bacteria can occur via other routes of transmission such via fomites, (inanimate objects such as countertops, cutting boards, utensils, towels, money, clothing, dishes, toys, dirt, excreted feces, diapers, surfaces in barns and stockyards, etc.), or via unpasteurized milk, dairy products, juices, etc.; or via contaminated water (e.g. drinking water, ponds and lakes, swimming pools, etc.); or via contaminated animals, meat, or produce; or fruits, etc.

In some aspects, the methods of the invention involve contacting pathogenic bacteria with the E. coli plasmid 01229_3 containing microcin. Accordingly, the invention provides i) substantially purified 0.1229_3 containing microcin protein; and ii) substantially pure cultures of bacteria that produce the microcin protein.

Proteins and Nucleic Acids

In some aspects the invention provides 0.1229_3 containing microcin protein and/or a gene that encodes the protein as well as proteins/polypeptides of the operon disclosed herein, and the genes which encode them.

Substantially purified 0.1229_3 containing microcin protein may be produced either recombinantly, or from a native or naturally occurring source such as the bacteria described herein. Those of skill in the art are familiar with techniques for genetically engineering organisms to recombinantly produce or overproduce a protein of interest such as 0.1229_3 containing microcin. Generally, such techniques involve excision of a gene encoding the protein from a natural source e.g. using nucleases or by amplifying the gene e.g. via PCR using primers complementary to sequences that flank the gene of interest. The gene can then be inserted into and positioned within a vector (e.g. an expression vector such as a plasmid or virus) so that it is able to be expressed (transcribed into translatable mRNA). Typically, the gene that is to be transcribed is juxtaposed to one or more suitable control elements such as promoters, enhancers, etc. that drive expression of the gene. Suitable vectors include but are not limited to plasmids, adenoviral vectors, baculovirus vectors (e.g. so-called shuttle or “bacmid” vectors, and the like). Suitable vectors may be chosen or constructed to contain appropriate regulatory sequences, including promoter sequences, terminator sequences, polyadenylation sequences, enhancer sequences, marker genes, and other sequences. The vectors may also contain a plasmid or viral backbone.

Typically, the vector is used to genetically engineer or infect a host organism where the gene is transcribed and translated into protein. In the host, the gene may be expressed from the vector (transcribed extrachromosomally, also called “in trans”) and may be overexpressed, i.e. expressed at a level that is higher than normally occurs in its native bacterial host. Alternatively, the gene may be inserted into the chromosome of the host (“in cis”). Exemplary expression systems that may be utilized include but are not limited to bacteria (such as E. coli), yeast, baculovirus, plant, mammalian, and cell-free systems. Host bacteria may be heterologous, i.e. they may be non-native bacteria in which the gene is not present in nature. Alternatively, they may be native bacteria that are natural hosts, but which are genetically engineered to produce the microcin in greater abundance (at higher levels or concentrations) than in the native, non-engineered host. Exemplary heterologous bacterial hosts include but are not limited to: various Lactobacillus species such as Lactobacillus casei, Lactobacillus acidophilus, Lactobacillus fermentum, Lactobacillus gasseri, Lactobacillus pentosus, Lactobacillus plantarum, Lactobacillus sporogenes, Lactobacillus brevis, Lactobacillus delbrueckii, Lactobacillus salivarius, Lactobacillus hilgardii, Lactobacillus lactis, Lactobacillus rhamnosus, Lactobacillus johnsonii, Lactobacillus leishmanis, Lactobacillus jensenii, Lactobacillus reuteri, Lactobacillus sakei, Lactobacillus cellobiosus, Lactobacillus crispatus, Lactobacillus curvatus, Lactobacillus caucasicus, and Lactobacillus helveticus, and others taught, for example, in United States patent application 20090169582 (Chua), the complete contents of which is hereby incorporated by reference in entirety; and other types of bacterial, fungal and/or viral recombinant hosts. Mammalian cells available in the art for heterologous protein expression include lymphocytic cell lines (e.g., NSO), HEK293 cells, Chinese hamster ovary (CHO) cells, COS cells, HeLa cells, baby hamster kidney cells, oocyte cells, and cells from a transgenic animal, e.g., mammary epithelial cell. For details, see Sambrook et al., Molecular Cloning: A Laboratory Manual, 2nd ed., Cold Spring Harbor Laboratory Press (1989). Many established techniques used with vectors, including the manipulation, preparation, mutagenesis sequencing, and transfection of DNA, are described in Current Protocols in Molecular Biology, Second Edition, Ausubel et al. eds., John Wiley & Sons (1992).

The vector or chromosome from which the microcin is transcribed includes at least a genetic sequence encoding the microcin described herein and may comprise one or more additional genes of the operon described herein, each of which encodes a respective protein or functional variant thereof (see below for explanation of “variant”. The one or more (at least one) gene(s) in the vector or chromosome is/are expressable and are operably (functionally, expressibly) linked to one or more control or expression elements, e.g. promoters, enhancers, etc. in a manner that facilitates, causes or allows expression of the gene(s). In some aspects, the genes are present on a plasmid such as the plasmid with the nucleotide sequence shown herein), or a plasmid with at least about 55, 60, 65, 70, 75, 80, 85, 90, or 95% or more (e.g. 96, 97, 98, 99%) identity

The protein that is produced is the E. coli plasmid 0.1229_3 containing microcin (or another protein encode by the operon as described above) or a physiologically active variant thereof. By “physiologically active variant” or “active variant” or “functional variant”, we mean a protein sequence that is able to kill pathogenic bacteria as described herein. The protein may have the sequence shown herein, or may include this sequence, or a sequence that shares at least about 95% identity to sequences herein (e.g. that is about 95, 96, 97, 98 or 99% identical thereto, as determined by alignment methods that are well-known), but that retains the ability to kill and/or impede growth/reproduction of and/or colonization by pathogenic bacteria. Compared to the wild type microcin, such variants are at least about 50%, and usually about 55, 60, 65, 70, 75, 80, 85, 90, or 95% or more as potent re killing, impeding growth and/or colonization, etc. In some embodiments, the variant may be more potent than the native microcin.

The variants of 0.1229_3 containing microcin that may be used in the practice of the invention may include those in which one or more amino acids are substituted by conservative or non-conservative amino acids, as is understood in the art. Further, deletions or insertions may also be tolerated without impairing the function. In addition, the microcin may be included in a chimeric or fusion protein that includes other useful sequences, e.g. tagging sequences (e.g. histidine tags), various targeting sequences (e.g. sequences that promote secretion or target the protein to a subcellular apartment or to the membrane), other antimicrobial sequences (e.g. other microcins), and the like, as well as spacer or linking sequences. The sequence of the microcin may be altered to prevent or discourage proteolysis, to promote solubility, or in any other suitable manner.

The invention also encompasses nucleic acid sequences that encode the microcin and active variants thereof as described herein. Variants, usually having at least about 95, 96, 97, 98, or 99% identity thereto, are also contemplated. However, those of skill in the art will recognize that the identity may be much lower (e.g. about 50, 55, 60, 65, 70, 75, 80, 85 or 90%) and the sequence may still encode a fully functional microcin, e.g. due to the redundancy of the genetic code.

Calculations of “homology” and/or “sequence identity” between two sequences may be performed as follows: The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference (native) sequence. The amino acid residues or nucleotides at corresponding amino acid positions or nucleotide positions are then compared. When a position the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In an exemplary embodiment, the percent identity between two amino acid sequences is determined using the Needleman and Wunsch (1970, J. Mol. Biol. 48:444-453) algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In an exemplary embodiment, the percent homology/identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that may be used if the practitioner is uncertain about what parameters may be applied to determine if a molecule is within a sequence identity, or homology limitation of the invention) are a Blossum 62 scoring matrix with a gap penalty of 12, a gap extend penalty of 4, and a frame shift gap penalty of 5. The percent identity/homology between two amino acid or nucleotide sequences can also be determined using the algorithm of E. Meyers and W. Miller ((1988) CABIOS, 4:11-17) that has been incorporated into the ALIGN program (version 2:0); using a PAM120 weight residue table, a gap length penalty of 12 and a gap penalty of 4.

The culturing and the maintenance of cultures of microorganisms such as the bacteria of the invention is carried out e.g. as described herein and as generally known in the art. Bacterial preparations may be lyophilized or freeze-dried.

The production of the substantially purified microcin protein is carried out by methods known to those of skill in the art, e.g. by collecting unpurified protein from a source such as the bacteria (or other expression system) that make the protein, and purifying and characterizing the protein using known steps, e.g. various separation techniques and identification techniques which include but are not limited to: centrifugation, column chromatography, affinity chromatography, electrophoresis, precipitation, sequencing, spectroscopy, etc. Preparations may be lyophilized or freeze-dried. By “substantially purified” we mean that the microcin is provided in a form that is at least about 75 wt. %, preferably at least about 80 wt. %, more preferably at least about 90 wt. %, and most preferably at least about 95 wt. % or more free from other macromolecules such as other peptides, proteins, nucleic acids, lipids, membrane fragments, etc., as is understood by those of skill in the art.

Compositions

The microcins and/or bacteria producing microcins (both of which may be referred to herein as “active agent(s) or “active ingredient(s))” of this invention will generally be used as a bactericidal active ingredient in a composition, i.e. a formulation, with at least one additional component such as a surfactant, a solid or liquid diluent, etc., which serves as a carrier. The formulation or composition ingredients are selected to be consistent with the physical properties of the active ingredient, the mode of application and environmental factors at the site of use, e.g. such as surface type, (e.g. soil or solid substrate, etc.), moisture, temperature, etc. If the composition is to be administered to a host, the ingredients are selected so as to be physiologically compatible with the host. Useful formulations include both liquid and solid compositions. Liquid compositions include solutions (including emulsifiable concentrates), suspensions, emulsions (including microemulsions and/or suspoemulsions) and the like, which optionally can be thickened into gels. The general types of aqueous liquid compositions are soluble concentrate, suspension concentrate, capsule suspension, concentrated emulsion, microemulsion and suspoemulsion. The general types of nonaqueous liquid compositions are emulsifiable concentrate, microemulsifiable concentrate, dispersible concentrate and oil dispersion.

The general types of solid compositions are dusts, powders, granules, pellets, pills, pastilles, tablets, films, filled or layered films, coatings, impregnations, gels, cakes, and the like, which can be water-dispersible (“wettable”) or water-soluble. Films and coatings formed from film-forming solutions or flowable suspensions may be useful for some applications. Active ingredients can be (micro) encapsulated and further formed into a suspension or solid formulation; alternatively, the entire formulation of active ingredient can be encapsulated (or “overcoated”). Encapsulation can control or delay release of the active ingredient. An emulsifiable granule combines the advantages of both an emulsifiable concentrate formulation and a dry granular formulation. High-strength compositions may be used as intermediates for further formulation.

Sprayable formulations are typically extended in a suitable medium before spraying. Liquid and solid formulations are formulated to be readily diluted in the spray medium, which may be aqueous based, e.g. water. Spray volumes can range from about one to several thousand liters, sprayable formulations may be tank mixed with water or another suitable medium for treatment by aerial or ground application, e.g. of stockyards, barns, stables, stalls, bins containing produce, etc. Smaller volume spray formulations for use on smaller surfaces (e.g. countertops, for application to small quantities of food stuffs, etc.) are also contemplated.

The formulations will typically contain effective amounts of active ingredient in the range of about 1 to about 99 percent by weight.

Solid diluents include, for example, clays such as bentonite, montmorillonite, attapulgite and kaolin, gypsum, cellulose, titanium dioxide, zinc oxide, starch, dextrin, sugars (e.g., lactose, sucrose), silica, talc, mica, diatomaceous earth, urea, calcium carbonate, sodium carbonate and bicarbonate, and sodium sulfate. Typical solid diluents are described in Watkins et al., Handbook of Insecticide Dust Diluents and Carriers, 2nd Ed., Dorland Books, Caldwell, N.J., the complete contents of which is hereby incorporated by reference in entirety.

Liquid diluents include, for example, water, N,N-dimethylalkanamides (e.g., N,N-dimethylformamide), limonene, dimethyl sulfoxide, N-alkylpyrrolidones (e.g., N-methylpyrrolidinone), ethylene glycol, triethylene glycol, propylene glycol, dipropylene glycol, polypropylene glycol, propylene carbonate, butylene carbonate, paraffins (e.g., white mineral oils, normal paraffins, isoparaffins), alkylbenzenes, alkylnaphthalenes, glycerine, glycerol triacetate, sorbitol, aromatic hydrocarbons, dearomatized aliphatics, alkylbenzenes, alkylnaphthalenes, ketones such as cyclohexanone, 2-heptanone, isophorone and 4-hydroxy-4-methyl-2-pentanone, acetates such as isoamyl acetate, hexyl acetate, heptyl acetate, octyl acetate, nonyl acetate, tridecyl acetate and isobornyl acetate, other esters such as alkylated lactate esters, dibasic esters and .gamma.-butyrolactone, and alcohols, which can be linear, branched, saturated or unsaturated, such as methanol, ethanol, n-propanol, isopropyl alcohol, n-butanol, isobutyl alcohol, n-hexanol, 2-ethylhexanol, n-octanol, decanol, isodecyl alcohol, isooctadecanol, cetyl alcohol, lauryl alcohol, tridecyl alcohol, oleyl alcohol, cyclohexanol, tetrahydrofurfuryl alcohol, diacetone alcohol and benzyl alcohol. Liquid diluents also include glycerol esters of saturated and unsaturated fatty acids (typically C₆-C₂₂), such as plant seed and fruit oils (e.g., oils of olive, castor, linseed, sesame, corn (maize), peanut, sunflower, grapeseed, safflower, cottonseed, soybean, rapeseed, coconut and palm kernel), animal-sourced fats (e.g., beef tallow, pork tallow, lard, cod liver oil, fish oil), and mixtures thereof Liquid diluents also include alkylated fatty acids (e.g., methylated, ethylated, butylated) wherein the fatty acids may be obtained by hydrolysis of glycerol esters from plant and animal sources, and can be purified by distillation. Typical liquid diluents are described in Marsden, Solvents Guide, 2nd Ed., Interscience, New York, 1950, the complete contents of which is hereby incorporated by reference in entirety.

The solid and liquid compositions of the present invention may include one or more surfactants. When added to a liquid, surfactants (also known as “surface-active agents”) generally modify, most often reduce, the surface tension of the liquid. Depending on the nature of the hydrophilic and lipophilic groups in a surfactant molecule, surfactants can be useful as wetting agents, dispersants, emulsifiers or defoaming agents. Surfactants can be classified as nonionic, anionic or cationic. Exemplary suitable surfactants can be found, for example, in United States patent application 20130143940 to Long, the entire contents of which is hereby incorporated by reference. Also useful for the present compositions are mixtures of nonionic and anionic surfactants or mixtures of nonionic and cationic surfactants. Nonionic, anionic and cationic surfactants and their recommended uses are disclosed in a variety of published references including McCutcheon's Emulsifiers and Detergents, annual American and International Editions published by McCutcheon's Division, The Manufacturing Confectioner Publishing Co.; Sisely and Wood, Encyclopedia of Surface Active Agents, Chemical Publ. Co., Inc., New York, 1964; and A. S. Davidson and B. Milwidsky, Synthetic Detergents, Seventh Edition, John Wiley and Sons, New York, 1987, the complete contents of each of which is hereby incorporated by reference in entirety.

Compositions of this invention may also contain formulation auxiliaries and additives, known to those skilled in the art as formulation aids (some of which may be considered to also function as solid diluents, liquid diluents or surfactants). Such formulation auxiliaries and additives may control: pH (buffers), foaming during processing (antifoams such polyorganosiloxanes), sedimentation of active ingredients (suspending agents), viscosity (thixotropic thickeners), in-container microbial growth (antimicrobials), product freezing (antifreezes), color (dyes/pigment dispersions), wash-off (film formers or stickers), evaporation (evaporation retardants), and other formulation attributes. Film formers include, for example, polyvinyl acetates, polyvinyl acetate copolymers, polyvinylpyrrolidone-vinyl acetate copolymer, polyvinyl alcohols, polyvinyl alcohol copolymers and waxes. Examples of formulation auxiliaries and additives include those listed in McCutcheon's Volume 2: Functional Materials, annual International and North American editions published by McCutcheon's Division, The Manufacturing Confectioner Publishing Co., the complete contents of which is hereby incorporated by reference in entirety.

The active agents described herein, and any other active ingredients are typically incorporated into the present compositions by dissolving or suspending the active ingredient in a solvent or by grinding in a liquid or dry diluent. Solutions, including emulsifiable concentrates, can be prepared by simply mixing the ingredients. The preparation may be lyophilized (freeze dried). If the solvent of a liquid composition intended for use as an emulsifiable concentrate is water-immiscible, an emulsifier is typically added to emulsify the active-containing solvent upon dilution with water. Active ingredient slurries, with particle diameters of up to 2,000 μm can be wet milled using media mills to obtain particles with average diameters below 3 μm. Aqueous slurries can be made into finished suspension concentrates (see, for example, U.S. Pat. No. 3,060,084, the complete contents of which is hereby incorporated by reference in entirety) or further processed by spray drying to form water-dispersible granules. Dry formulations usually require dry milling processes, which produce average particle diameters in the 2 to 10 μm range. Dusts and powders can be prepared by blending and usually grinding (such as with a hammer mill or fluid-energy mill). Granules and pellets can be prepared by spraying the active material upon preformed granular carriers or by agglomeration techniques. See Browning, “Agglomeration”, Chemical Engineering, Dec. 4, 1967, pp 147-48, Perry's Chemical Engineer's Handbook, 4th Ed., McGraw-Hill, New York, 1963, pages 8-57 and following, and WO 91/13546. Pellets can be prepared as described in U.S. Pat. No. 4,172,714. Water-dispersible and water-soluble granules can be prepared as taught in U.S. Pat. Nos. 4,144,050, 3,920,442 and DE 3,246,493. Tablets can be prepared as taught in U.S. Pat. Nos. 5,180,587, 5,232,701 and 5,208,030. Films can be prepared as taught in GB 2,095,558 and U.S. Pat. No. 3,299,566. For further information regarding the art of formulation, see T. S. Woods, “The Formulator's Toolbox-Product Forms for Modern Agriculture” in Pesticide Chemistry and Bioscience, The Food-Environment Challenge, T. Brooks and T. R. Roberts, Eds., Proceedings of the 9th International Congress on Pesticide Chemistry, The Royal Society of Chemistry, Cambridge, 1999, pp. 120-133. See also U.S. Pat. No. 3,235,361, Col. 6, line 16 through Col. 7, line 19 and Examples 10-41; U.S. Pat. No. 3,309,192, Col. 5, line 43 through Col. 7, line 62 and Examples 8, 12, 15, 39, 41, 52, 53, 58, 132, 138-140, 162-164, 166, 167 and 169-182; U.S. Pat. No. 2,891,855, Col. 3, line 66 through Col. 5, line 17 and Examples 1-4; Klingman, Weed Control as a Science, John Wiley and Sons, Inc., New York, 1961, pp 81-96; Hance et al., Weed Control Handbook, 8th Ed., Blackwell Scientific Publications, Oxford, 1989; and Developments in formulation technology, PJB Publications, Richmond, U K, 2000. The complete contents of each of these references is hereby incorporated by reference in entirety.

In addition, the formulations may include other suitable active agents, e.g. other antimicrobial agents such as other microcins, antibiotics, etc.; or broadly defined antimicrobials such as antiseptics or heavy metals, etc.

Incorporation into Various Products

The active agents described herein may be incorporated into and/or used as an amendment to many different products, e.g. substrates and media which include but are not limited to: so-called “hand-sanitizing” preparations and soaps, gels, etc.; various sprays and washes; detergents and various cleaning agents; fabrics e.g. linings for materials such as diapers and other garments that may be contacted by feces; “booties” that are used to cover and protect shoes; disposable or non-disposable gloves; disposable or non-disposable food preparation surfaces, e.g. as sheets of material that can be placed on a cutting surface, or in a cutting surface itself; in storage apparatuses for implements used in food preparation (e.g. knife blocks, or holders, etc.); and others.

In some aspects, the active agents described herein are incorporated into packaging materials, e.g. packaging materials designed to contain meat or meat products or produce. For example, the packaging material may be impregnated with the active agent either during or after manufacture or may be coated onto one or more surfaces of the material. The packaging material may be a film e.g. formed from a flexible polymer that may be transparent, or may be a rigid or semi-rigid container formed from e.g. plastic resin, styrofoam, wood, cardboard or pasteboard or other molded cellulose product, or made from some other so-called “natural” material. The packaging material may be in the form of “peanuts”. The material may be biodegradable. United States patent applications 20120259295 (Bonutti) and 20030234466 (Rasmussen) and references cited therein, the complete contents of all of which are hereby incorporated by reference in entirety, discuss the preparation of various types of packaging materials.

### Methods and Uses

In some aspects, the invention provides methods of using the microcins and bacteria that produce the microcins described herein, for preventing or decreasing the transmission of pathogenic Escherichia coli (E. coli) bacteria from a first location to a second location, e.g. from a first host (that may or not be a susceptible host) or first contaminated area, to a second host or previously uncontaminated area. The second host may or may not be susceptible. The first location may be a “reservoir” host or area/location that is already colonized by the pathogenic bacteria. Alternatively, the first host or location may be likely to be colonized or possible to colonize.

### Compositions and Pharmaceutical Compositions

Antibodies

A plasmid 0.1229_3 containing microcin immunogen typically is used to prepare antibodies by immunizing a suitable subject, (e.g., rabbit, goat, mouse or other mammal) with the immunogen. An appropriate immunogenic preparation can contain, for example, a naturally occurring or recombinantly expressed plasmid 0.1229_3 containing microcin polypeptide or a chemically synthesized plasmid 0.1229_3 containing microcin peptide. The preparation can further include an adjuvant, such as Freund's complete or incomplete adjuvant, or similar immunostimulatory agent. Immunization of a suitable subject with an immunogenic plasmid 0.1229_3 containing microcin preparation induces a polyclonal anti-plasmid 0.1229_3 containing microcin antibody response. Such methods are known in the art.

Hence, polyclonal anti-plasmid 0.1229_3 containing microcin antibodies can be prepared as described above by immunizing a suitable subject with a plasmid 0.1229_3 containing microcin immunogen. If desired, the antibody molecules directed against the plasmid 0.1229_3 containing microcin polypeptide can be isolated from a mammal (e.g., from the blood) and further purified by well-known techniques, such as protein A chromatography to obtain the IgG fraction. At an appropriate time after immunization, e.g., when the anti-plasmid 0.1229_3 containing microcin antibody titers are highest, antibody-producing cells can be obtained from the subject and used to prepare monoclonal antibodies by standard techniques, such as the hybridoma technique originally described by Kohler and Milstein (1975) Nature 256:495-497 (see also, Brown et al. (1981) J. Immunol 127:539-46; Brown et al. (1980) J. Biol. Chem. 255:4980-83; Yeh et al. (1976) Proc. Natl. Acad. Sci. USA 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, N.Y. (1980); Lerner, E. A. (1981) Yale J. Biol. Med. 54:387-402; Gefter, M. L. et al. (1977) Somatic Cell Genet., 3:231-36). Briefly, an immortal cell line (typically a myeloma) is fused to lymphocytes (typically splenocytes) from a mammal immunized with a plasmid 0.1229_3 containing microcin immunogen as described above, and the culture supernatants of the resulting hybridoma cells are screened to identify a hybridoma producing a monoclonal antibody that binds specifically to a plasmid 0.1229_3 containing microcin polypeptide.

Accordingly, in embodiments, the present invention is also drawn to antibodies which selectively bind to a polypeptide that is a plasmid 0.1229_3 containing microcin polypeptide. In view of the well-established principle of immunologic cross-reactivity, the present invention therefore contemplates antigenically related variants of the polypeptide. An “antigenically related variant” is a polypeptide that includes at least a six amino acid residue sequence portion of a polypeptide and which is capable of inducing antibody molecules that immunoreact with a polypeptide of antibodies can be synthetic, monoclonal, or polyclonal and can be made by techniques well known in the art.

The plasmid 0.1229_3 containing microcin polypeptide or even a cell of the invention can be incorporated into a composition or a pharmaceutical composition which are suitable for administration. Accordingly, the present invention contemplates a composition comprising a polypeptide according to the invention or a cell according to the invention.

In a further preferred embodiment, the composition is an aqueous composition.

In a further embodiment, the composition has antimicrobial, antibacterial or antitumoral activity. Then, the composition inhibits bacterial adherence to human intestinal epithelial cells.

In a preferred embodiment, the composition is a probiotic composition. Preferably, the probiotic composition comprises a mixture of probiotic microorganisms, preferably of E. coli DSM 17252. For example, the composition comprises cells and/or autolysates of at least one of E. coli G1/2 (DSM 16441), G3/10 (DSM 16443), G4/9 (DSM 16444), G5 (DSM 16445), G6/7 (DSM 16446) and G8 (DSM 16448) or a mixture thereof. A composition may contain autolysates as well as cells in an amount of 3.00×10⁶ to 2.25×10⁸ cells per 1 ml, preferably 1.5-4.5×10⁷ cells per 1 ml. Preferably, the composition comprises autolysates as well as cells of E. coli bacteria and further additives. Accordingly, the invention provides a probiotic composition comprising at least one of the bacterial strains E. coli G1/2 (DSM 16441), G3/10 (DSM 16443), G4/9 (DSM 16444), G5 (DSM 16445), G6/7 (DSM 16446) and G8 (DSM 16448), i.e., one of the strains mentioned above or any bacterial strain selected by the method of the invention, where the composition comprises at least 1 strain, preferably from 2 to 3 strains, more preferably from 2 to 4 strains, even more preferred from 2 to 5 strains and most preferred from 2 to 6 strains, and where each of the strains is present in the composition in a proportion from 0.1% to 99.9%, preferably from 1% to 99%, more preferably from 10% to 90%. In a preferred embodiment, the composition comprises at least one of the bacterial strains of the invention together with another strain or mixture of strains where the mixture comprises preferably from 2 to 6 strains, more preferably from 2 to 4 strains, most preferably from 2 to 3 strains and where each of the strains is present in the composition in a proportion from 0.1% to 99.9%, preferably from 1% to 99%, more preferably from 10% to 90%. The probiotic compositions of the invention are preferably in a lyophilized form, in a frost form or even dead. In a preferred embodiment, a probiotic composition comprises one or more probiotic microorganisms and a carrier which functions to transport the one or more probiotic microorganisms to the gastrointestinal tract, the carrier may comprise modified or unmodified resistant starch in the form of high amylose starches or mixtures thereof The carrier acts as a growth or maintenance medium for microorganisms in the gastrointestinal tract such that the probiotic microorganisms are protected during passage to the large bowel or other regions of the gastrointestinal tract.

In a preferred embodiment, the composition may further be a pharmaceutical composition. Then, the pharmaceutical composition further comprises a pharmaceutically acceptable carrier. The pharmaceutical composition may contain a therapeutically effective amount of the polypeptide or the cell and one or more adjuvants, excipients, carriers, and/or diluents. Acceptable diluents, carriers and excipients typically do not adversely affect a recipient's homeostasis (e.g., electrolyte balance). Acceptable carriers include biocompatible, inert or bioabsorbable salts, buffering agents, oligo- or polysaccharides, polymers, viscosity-improving agents, preservatives and the like. Further details on techniques for formulation and administration of pharmaceutical compositions can be found in, e.g., REMINGTON′S PHARMACEUTICAL SCIENCES (Maack Publishing Co., Easton, Pa.).

Example of additives include glucose, lactose, sucrose, mannitol, starch, cellulose or cellulose derivatives, magnesium stearate, stearic acid, sodium saccharin, talcum, magnesium carbonate and the like. Examples of additives that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets.

In embodiments, supplementary active compounds can also be incorporated into the compositions.

Pharmaceutical compositions of the invention are formulated to be compatible with its intended route of administration. Examples of routes of administration include parenteral, e.g., intravenous, intradermal, subcutaneous, oral, transdermal (topical), transmucosal, and rectal administration.

Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Oral administration may be applied in the form of a capsule, liquid, tablet, pill, or prolonged release formulation.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier, wherein the compound in the fluid carrier is applied orally and swished and expectorated or swallowed. Pharmaceutically compatible binding agents, and/or adjuvant materials can be included as part of the composition. The oral composition can contain any of the following ingredients, or compounds of a similar nature: a salt such as sodium chloride or magnesium sulfate, such as magnesium sulfate.? H₂O, potassium chloride, calcium chloride, such as calcium chloride.2 H₂O, magnesium chloride, such as magnesium chloride.6 H₂O, purified water, a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The polypeptide; the cell or the composition, may be administered to a subject in a pharmaceutically effective amount. Administration of a pharmaceutically effective amount of the polypeptide; the cell or the compositions of the present invention is defined as an amount effective, at dosages and for periods of time necessary to achieve the desired result. For example, a pharmaceutically effective amount of a polypeptide; a cell or a composition may vary according to factors such as the disease state, age, sex, and weight of the individual, and the ability of the polypeptide; the cell or the compositions to elicit a desired response in the individual. Dosage regime may be adjusted to provide the optimum therapeutic response. For example, several divided doses may be administered daily, or the dose may be proportionally reduced as indicated by the exigencies of the therapeutic situation.

In order to optimize therapeutic efficacy, a the microcin is administered at different dosing regimens at different points of time.

The subject can be administered a single pharmaceutically effective dose or multiple pharmaceutically effective doses, e.g., 2, 3, 4, 5, 6, 7, or more. Specifically, the subject may be administered a single pharmaceutically effective dose 2, 3, 4, 5, 6, 7, or more times a day.

Specifically, the subject can be administered a dose of 5-15 droplets of an aqueous composition. More preferably a dose of 10 droplets are to be administered. In such an embodiment, 1 ml contains about 14 droplets.

A pharmaceutically effective amount (i.e., a pharmaceutically effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

Administrations of multiple doses can be separated by intervals of hours, days, weeks, or months. In further embodiments, they are administered at least one time, two times or three times a day with meals within water, preferably three times a day with meals within water.

They can optionally be administered to the subject for a limited period of time and/or in a limited number of doses. For example, in some embodiments administration to the subject can be terminated (i.e., no further administrations provided) within, e.g., one year, six months, one month, or two weeks. For example, the provided administration may be terminated after six months. In chronic diseases, it may be necessary to increase the period to up to six months.

In some embodiments, the dose may be increased after 2 days, 3 days, 4 days, 5 days, 6 days, 1 week, 2 weeks, three weeks, four weeks or more. The dose to be administered to a subject may be increased to 15-25 droplets, more preferably to 20 droplets, of an aqueous composition.

In children, they may be administered in an oral form of 5-15 droplets of an aqueous composition. More preferably 10 droplets are to be administered to a child. In further embodiments, they are administered to a child at least one time, two times or three times a day with meals within water, preferably one time a day with meals within water.

In all medical use embodiments, the polypeptide; the cell; the composition or the kit of the invention, are to be administered at the beginning of the treatment to an adult 10 droplets three times a day with meals within water and the dose is increased after 1 week to 20 droplets and to a child 10 droplets once per day with meals within water.

### Application to Surfaces

Those of skill in the art will recognize that it is also beneficial to prevent (discourage, impede, lessen, decrease, etc.) transmission of pathogenic bacteria from non-host sources to possible hosts, e.g. to prevent transmission from surfaces or areas which harbor the pathogens. The invention also comprises methods of doing so by applying the microcin of the invention and/or bacteria encoding the microcin, to surfaces which harbor the pathogens, or which are suspected of harboring the pathogens, or which could become contaminated with pathogens. Applying or treating such surfaces may be accomplished by any of many methods, e.g. by spraying a preparation of the microcin or bacteria, by applying a composition comprising a powder or granules, etc. Suitable compositions are described above. In general, the amount of microcin that is applied to a surface in order to be effective is in the range of from between about 1 ug and 100 mg; and the amount of bacteria that is applied is in the range of from about 10³ to about 10¹², and is preferably in the range of from about 10⁶ to about 10⁹.

Areas that are particularly prone to contamination with pathogenic bacteria include those which house of livestock or fowl. Such areas, especially commercial areas, may be treated using the compositions of the invention, especially spray formulations. The areas may or may not be associated with a commercial enterprise, e.g. they may be associated with for profit or non-profit farms, stables, etc. The areas may also be set aside for animals e.g. as reserves, zoos, stockyards etc., or may be located at veterinary facilities. The compositions of the invention may be applied to any suitable surface where the microcin may be useful to kill pathogenic bacteria, e.g. soil or grass, flooring, stalls, pens, milking carousels, feed lot surfaces, drinking and/or feeding containers, cages, crates, truck beds, etc. Exemplary animals which are housed in such areas and are potential hosts of pathogenic bacteria include but are not limited to: livestock e.g. horses, mares, mules, jacks, jennies, colts, cows, calves, yearlings, bulls, oxen, sheep, goats, lambs, kids, hogs, shoats, pigs, bison, and others; and avian species such as land and water fowl e.g. chickens, turkeys, ducks, geese, ostriches, guinea fowl, etc. The preparations of the invention may be applied to the animals themselves, or to specific areas of the animals, e.g. to feet, the anal area, etc.

In addition, the preparations of the invention may be applied to various products, especially products derived from animals that are susceptible to infection with and/or to disease caused by pathogenic bacteria. The preparations may be applied to or included in (mixed into), for example, meats or meat products (including both raw and so-called “ready to eat” meat and poultry products), eggs, hides, carcasses, horns, hooves, feathers, etc.

### Diseases Prevented or Treated

The types of diseases and conditions that may be prevented or treated using the methods and compositions disclosed herein include any of those which are caused by pathogenic E. coli, including but are not limited to: food poisoning (e.g. in humans), gastroenteritis, diarrhea, urinary tract infections, neonatal meningitis, hemolytic-uremic syndrome, peritonitis, mastitis, septicemia and Gram-negative pneumonia, shigellosis, dysentery, etc. In some aspects, probiotic preparations are contemplated, e.g. liquid or solid preparations that are taken prophylactically to prevent or treat disease symptoms or so-called Traveler's diarrhea prior to or during travel.

Herein, where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

It is understood that modifications which do not substantially affect the activity the various embodiments of this invention are also provided within the definition of the invention provided herein. Accordingly, the following examples are intended to illustrate but not limit the present invention.

### EXAMPLES

The inventions being thus described, it will be obvious that the same may be varied in many ways. Such variations are not to be regarded as a departure from the spirit and scope of the inventions and all such modifications are intended to be included within the scope of the following claims. The above specification provides a description of the manufacture and use of the disclosed compositions and methods. Since many embodiments can be made without departing from the spirit and scope of the invention, the invention resides in the claims. The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilized for realizing the invention in diverse forms thereof.

A microcin amplifies Shiga toxin 2a (Stx2a) production of Escherichia coli O157:H7

Escherichia coli O157:H7 is a foodborne pathogen, implicated in various multi-state outbreaks. It encodes Shiga toxin on a prophage, and Shiga toxin production is linked to phage induction. An E. coli strain, designated 0.1229, was identified that amplified Stx2a production when co-cultured with E. coli O157:H7 strain PA2. Growth of PA2 in 0.1229 cell-free supernatants had a similar effect, even when supernatants were heated to 100° C. for 10 min, but not after treatment with Proteinase K. The secreted molecule was shown to use TolC for export and the TonB system for import. The genes sufficient for production of this molecule were localized to a 5.2 kb region of a 12.8 kb plasmid. This region was annotated, identifying hypothetical proteins, a predicted ABC transporter, and a cupin superfamily protein. These genes were identified and shown to be functional in two other E. coli strains, and bioinformatic analyses identified related gene clusters in similar and distinct bacterial species. These data collectively suggest E. coli 0.1229 and other E. coli produce a microcin that induces the SOS response in target bacteria

### Introduction

E. coli O157:H7 is a notorious member of the enterohemorrhagic E. coli (EHEC) pathotype, which causes hemolytic colitis and hemolytic uremic syndrome (HUS) through production of virulence factors including the locus of enterocyte effacement (LEE) and Shiga toxins (Stx) (1, 2). Stx is encoded on a lambdoid prophage (3). Induction of the prophage and subsequent upregulation of stx is tied to activation of the bacterial SOS response (4). Therefore, DNA damaging agents including certain antibiotics increase Stx synthesis, and are typically counter indicated during treatment (5). There are two Stx types, referred to as Stx1 and Stx2 (6). Stx1 is further divided into three subtypes, Stx1a, Stx1c and Stx1d (7). Stx2 also has multiple subtypes, designated Stx2a, Stx2b, Stx2c, Stx2d, Stx2e, Stx2f, Stx2g (7), Stx2h (8) and Stx2i (9). In general, infections caused by Stx1, and interestingly, even those with both Stx1 and Stx2 (such as strains EDL933 (10) and Sakai (11)) are associated with less severe disease symptoms than Stx2-only producing E. coli (12-14). Of the Stx2 subtypes, Stx2a is more commonly associated with clinical cases and instances of HUS (14-17). Indeed, the FAO and WHO considers STEC carrying stx2a to be of greatest concern (18).

Stx2a levels can be affected in vitro and in vivo when E. coli O157:H7 is cultured along with other bacteria. Indeed, it was found that stx2a expression is downregulated by various probiotic species (19, 20) or in a media conditioned with human microbiota (21). Conversely, non-pathogenic E. coli that are susceptible to infection by the stx2a-converting phage were reported to increase Stx2a levels (22, 23). This mechanism is O157:H7 strain-dependent (23), and requires expression of the E. coli BamA, which is the phage receptor (24, 25).

Production of Stx2a by 0157:H7 can also increase in response to molecules secreted by other members of the gut microbiota (24, 26), such as bacteriocins and microcins. Bacteriocins are proteinaceous toxins produced by bacteria that inhibit the growth of closely related bacteria. For example, a colicin E9 (ColE9) producing strain amplified Stx2a when grown together with Sakai to higher levels than a colicin E3 (ColE3) producing strain (26). ColE9 is a DNase, while ColE3 has RNase activity, and this may explain the differences in SOS induction and Stx2a levels. In support of this, the addition of extracted DNase colicins to various E. coli O157:H7 strains increased Stx2a production, but not Stx1 (26). Additionally, microcin B17 (MccB17), a DNA gyrase inhibitor, was shown to amplify Stx2a production (24).

### Results

### 0.1229 Amplifies Stx2a Production in a Cell Independent Manner

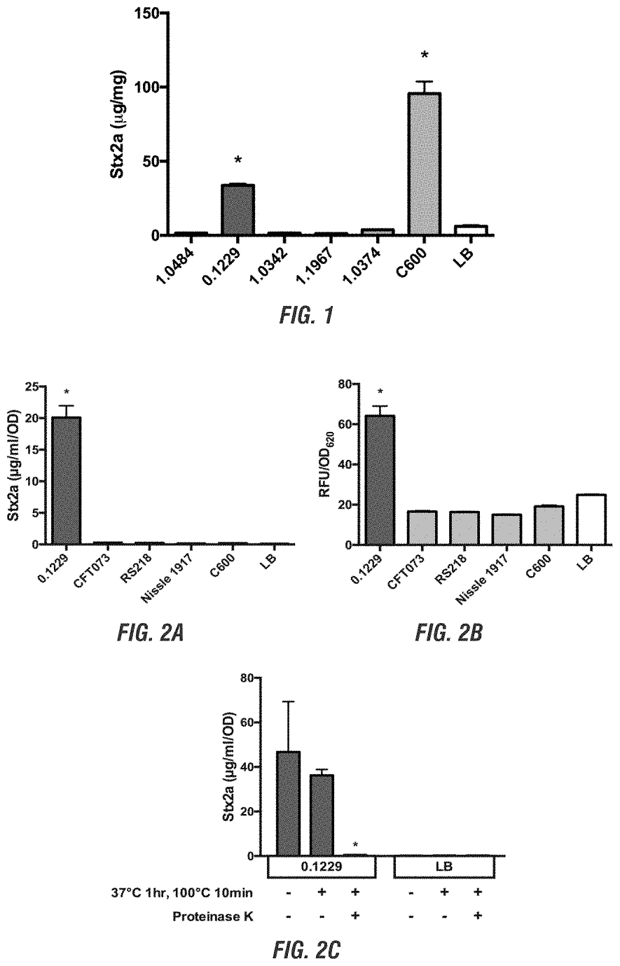

Human-associated E. coli isolates were tested for their ability to enhance Stx2a production in co-culture with O157:H7. Strain 0.1229 significantly increased Stx2a production of PA2, compared to PA2 alone (FIG. 1). C600 was included as a positive control, as it was previously shown to increase Stx2a production when co-cultured with O157:H7 (22, 23).

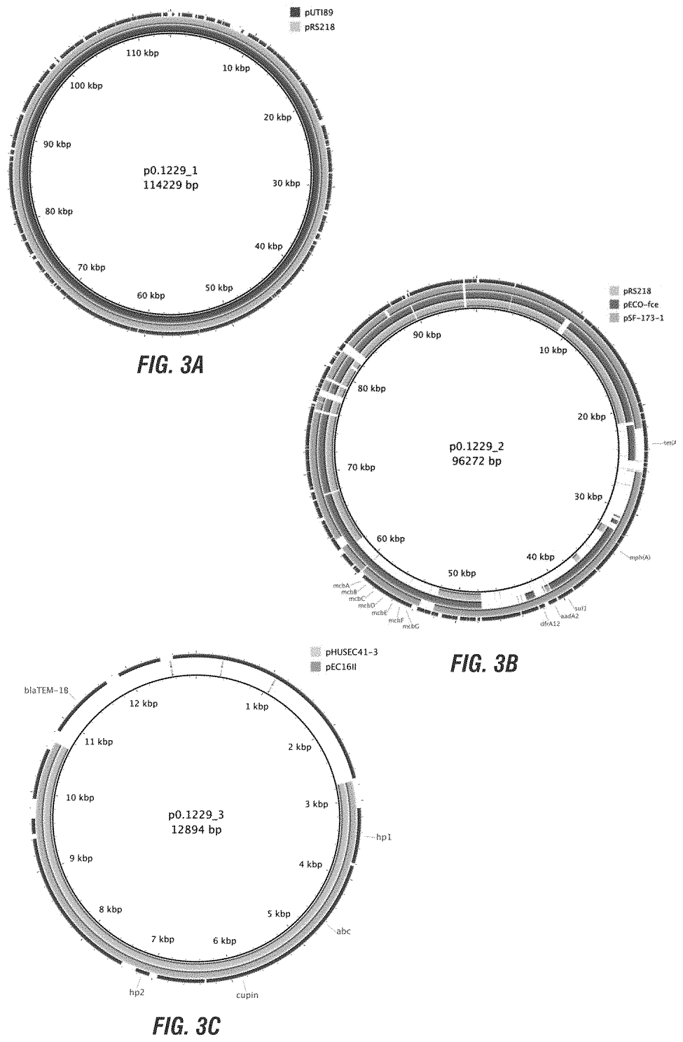

Growth of PA2 in cell-free supernatants of 0.1229 also amplified Stx2a production, indicating this phenomenon does not require whole cells (FIG. 2A). Sequencing of the genome of 0.1229 using Illumina technology revealed that it belonged to the same sequence type (ST73) as E. coli strains CFT073 (27) and Nissle 1917 (28), and carried a plasmid similar to pRS218 in E. coli RS218 (29). However, supernatants harvested after growth of these strains failed to increase Stx2a production by PA2 (FIG. 2A). To test whether increased Stx2a production was dependent on recA, 0.1229 was co-cultured with the W3110ΔtolC PrecA-gfp reporter strain. As anticipated, we found that among this collection, only strain 0.1229 increased GFP expression as a co-culture (FIG. 2B). Treatment of 0.1229 supernatants revealed this bioactivity was resistant to boiling, and sensitive to Proteinase K (FIG. 2C).

### The Plasmids of 0.1229 may Play a Role in Stx2a Amplification

Further analysis of the Illumina sequence data revealed high sequence identity between the chromosomes of 0.1229, CFT073, Nissle 1917 and RS218 (data not shown). The most notable differences were in predicted plasmid content. To obtain a more complete picture, PacBio long read technology was used to sequence the genome of 0.1229.

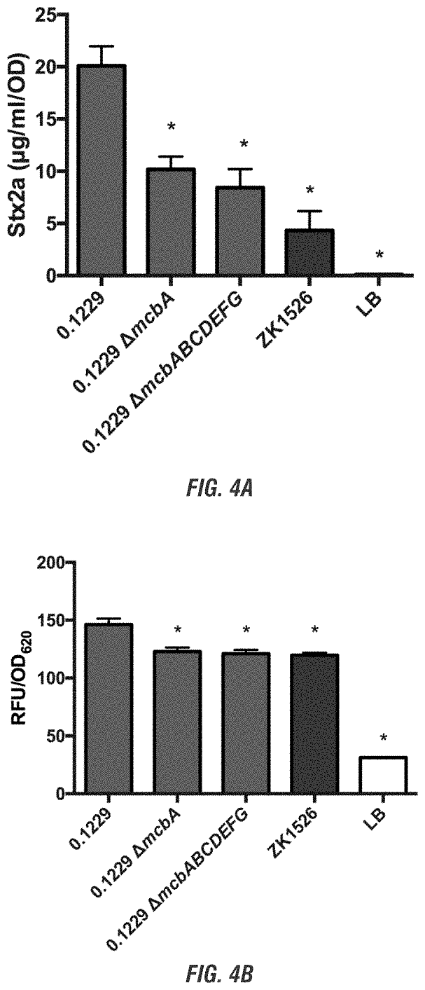

The largest plasmid of 0.1229, designated p0.1229_1, was 114,229 bp, and 99.99% identical with 100% query coverage to pUTI89 (30) and pRS218 (29) (FIG. 3A). A second plasmid designated p0.1229_2 had five identifiable antimicrobial resistance genes, was 96,272 bp, and encoded the operon for microcin B17 (MccB17), a microcin that inhibits DNA gyrase (31). The plasmid p0.1229_2 shared high sequence identity with other known plasmids, being 99.96% identical with 57% query to pRS218, 97.81% identical with 82% query to pECO-fce (NCBI accession CP015160) and 100% identical with 89% query to pSF-173-1 (32) (FIG. 3B). A third plasmid was smaller than the cutoff for size selection used during PacBio library preparation but was closed by Illumina sequencing. This plasmid, designated p0.1229_3, was 12,894 bp and encoded ampicillin resistance (blaTEM-1b) (FIG. 3C). It was similar to pEC16II (NCBI accession KU932034), 99.85% identical with 61% query, and to pHUSEC41-3 (33), 98.89% identical with 61% query.

As RS218 did not amplify Stx2a production (FIG. 2A), it was assumed that p0.1229_1 did not encode the genes responsible for this phenotype. Similarly, E. coli strain SF-173 did not increase GFP when co-cultured with W3110ΔtolC PrecA-gfp, suggesting genes encoded on p0.1229_2 were also not required (data not shown).

Strain 0.1229 Encodes Microcin B17, which is Partially Responsible for Stx2a Amplification

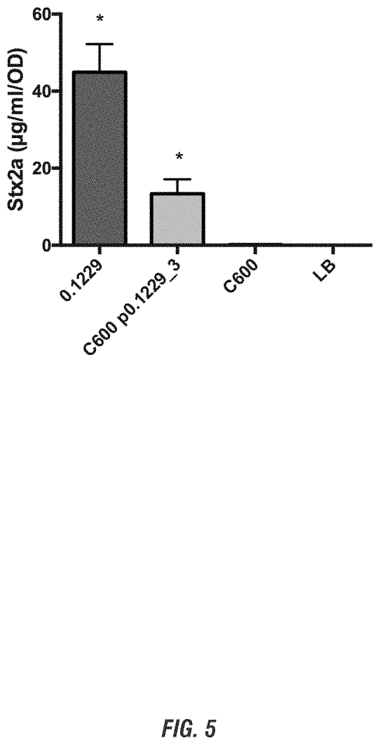

Microcin B17 (MccB17) is a 3.1 kDa (43 amino acid) DNA gyrase inhibitor that is found on a seven gene operon, with mcbA encoding the 69 amino acid microcin precursor (31). Although pSF-173-1 encodes this operon, there was a three-nucleotide deletion observed in mcbA in pSF-173-1, compared to an earlier published sequence (31). This deletion is predicted to shorten a ten Gly homopolymeric stretch by one amino acid residue. Although this Gly rich region is not important for interaction with the gyrase-DNA complex (34), it seemed prudent to confirm that the results reported above with strain SF-173 were not due to production of a non-functional McbA. Therefore, knockouts of mcbA (AmcbA), and the entire operon (AmcbABCDEFG) were constructed in 0.1229. These mutations decreased Stx2a amplification by 0157:H7 compared to wildtype 0.1229 (FIG. 4A) however they did not ablate the Stx2a levels back to mono-culture levels. Similar results were seen with the PrecA-gfp strain (FIG. 4B), although differences were less pronounced than those seen with the Stx assays.

### Four ORFs Encoded by p0.1229_3 are Necessary for Stx2a Amplification Phenotype

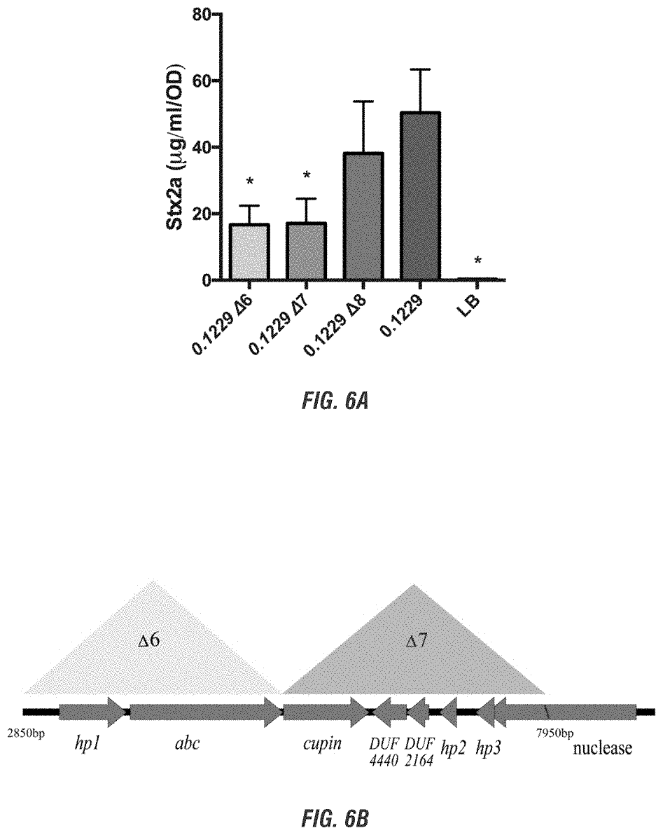

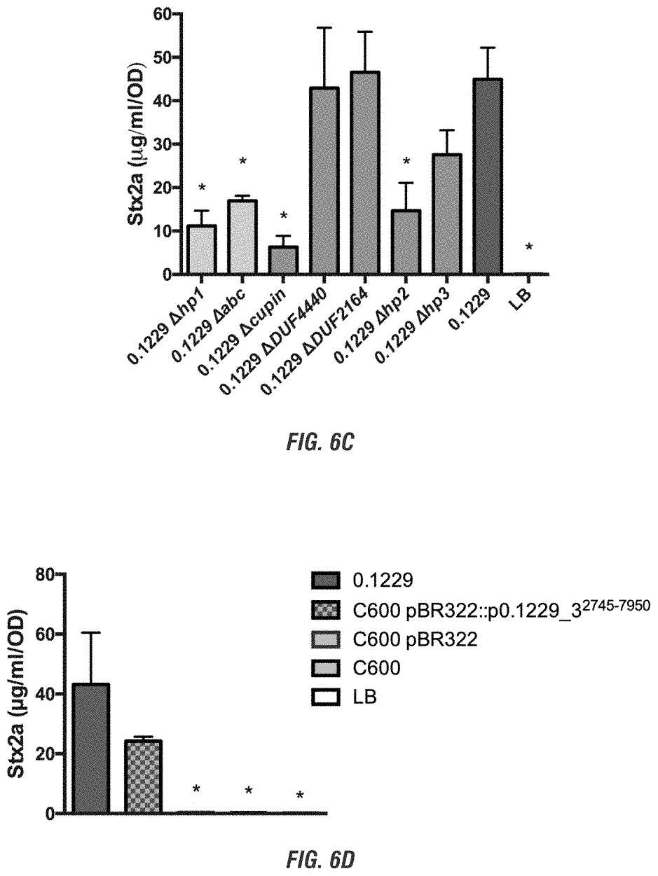

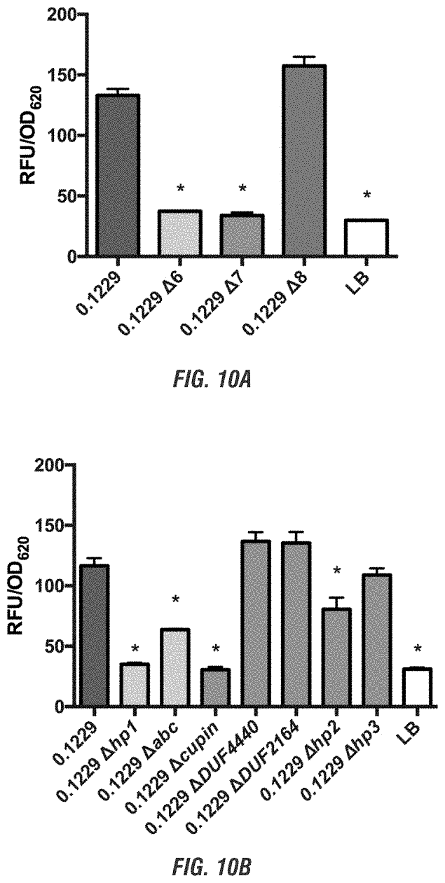

It was next hypothesized that p0.1229_3 encoded the activity responsible for Stx2a amplification by 0.1229ΔmcbA and 0.1229ΔmcbABCDEFG. A C600 strain transformed with p0.1229_3 amplified Stx2a production of PA2 (FIG. 5), confirming the importance of this plasmid. By systematically deleting portions of p0.1229_3, two regions were identified as essential for increased Stx2a production (FIG. 6A). The genes annotated in these regions are referred to as hypothetic proteins (hp), domains of unknown (DUF), an ATP-binding cassette (ABC)-type transporter and a member of the cupin superfamily of conserved barrel domains. The mutant, 0.1229 Δ6 (2850-5473 bp), deleted two open reading frames (ORFs), referred to as hp1 and ABC, and 0.1229 Δ7 (5426-7950 bp) deleted cupin, DUF4440, DUF2164, hp2, hp3 and a portion of a nuclease (FIG. 6B). These results were confirmed using co-culture assays with the PrecA-gfp reporter (FIG. 10). Insertional inactivation of individual ORFs in these regions identified four, hp1, abc, cupin, and hp2, that were necessary for enhanced Stx2a production (FIG. 6C). Similar results were shown in co-culture with PrecA-gfp, although 0.1229Δhp2 showed only a moderate decrease in GFP expression (FIG. 10). Cloning of a 5.2kb region of the plasmid, spanning upstream of hp1 through the beginning of the nuclease-encoding gene, confirmed this activity is encoded within this region (FIG. 6D). Cloning of a similar region that ended after abc did not provide C600 the ability to increase GFP (data not shown).

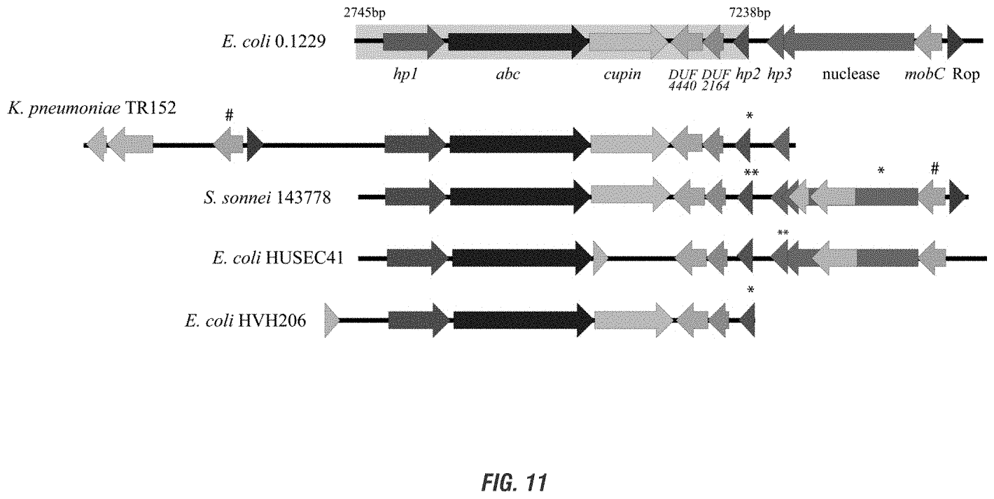

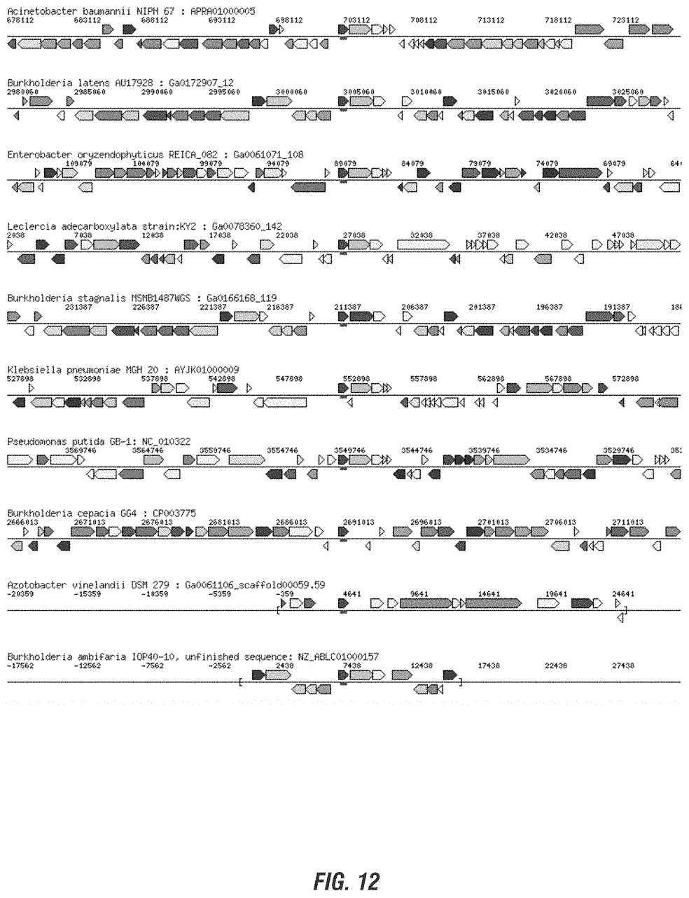

In silico comparisons identified a nearly identical gene cluster in other species, including Shigella sonnei and Klebsiella pneuomoniae (FIG. 11). The region of p0.1229_3 spanning nucleotides 2745 to 7238bp was greater than 99.6% identical on the nucleotide level, when comparing all the strains in FIG. 11. Similar gene clusters containing Hp1 at 36 to 68% amino acid identity, were found in other species as well (FIG. 12). In these clusters, orthologs to hp1, abc, and cupin were commonly co-localized and the genes were found in the same order.

The Secreted Molecule Requires tolC for Secretion, and tonB for Import into Target Strains

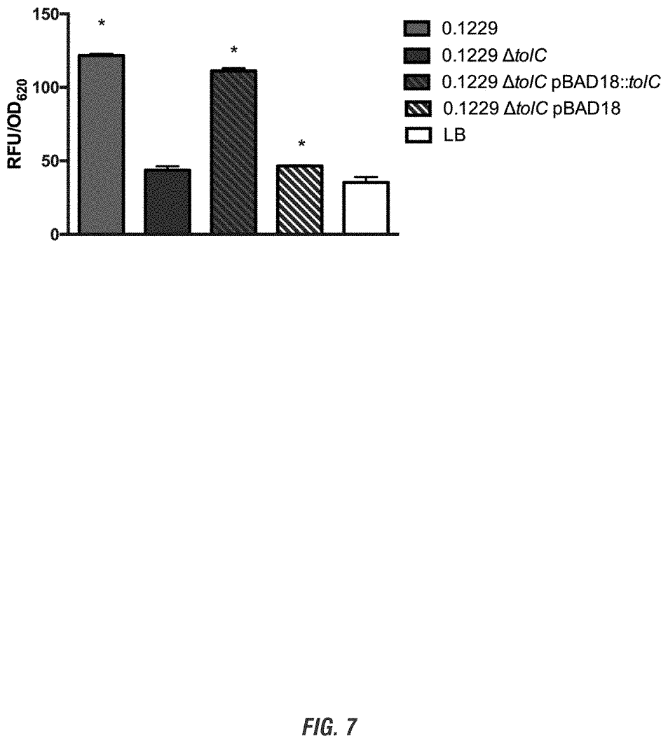

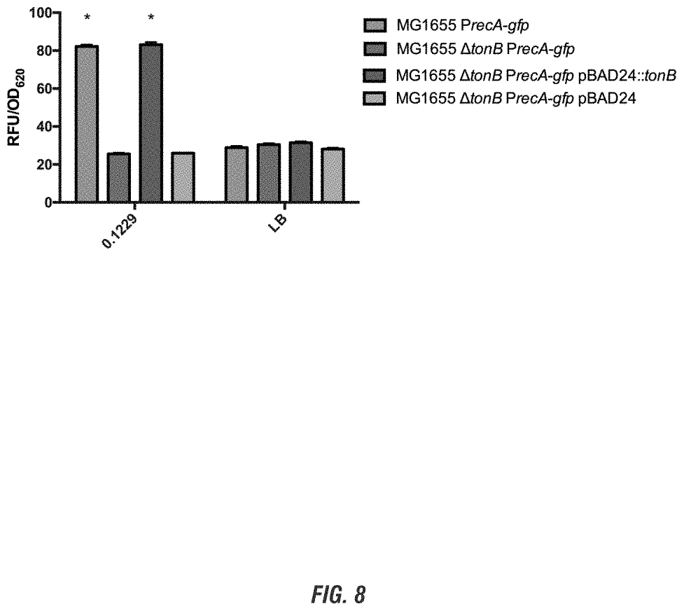

Some bacteriocins and microcins require genes encoded outside of the main operon for secretion, such as the efflux protein TolC (35). The supernatant of 0.1229Δto/C did not increase Stx2a expression by strain PA2 to levels seen with wildtype 0.1229 supernatants (data not shown). Similar results were observed in co-culture experiments using the PrecA-gfp carrying strain (FIG. 8). The phenotype was restored when to/C was complemented on a plasmid, but only when tested with the PrecA-gfp strain (FIG. 7). Similarly, numerous bacteriocins are translocated into target cells using the TonB system (36). A tonB knockout was constructed in the PrecA-gfp reporter strain, as we were unsuccessful generating this in a O157:H7 background. In co-culture with 0.1229, the MG1655ΔtonB PrecA-gfp strain produced lower GFP levels than the MG1655 PrecA-gfp strain (FIG. 8). This phenotype was restored when pBAD24::tonB, but not pBAD24, was transformed into the mutant strain (FIG. 8).

### The Gene Cluster was Identified in Additional Strains

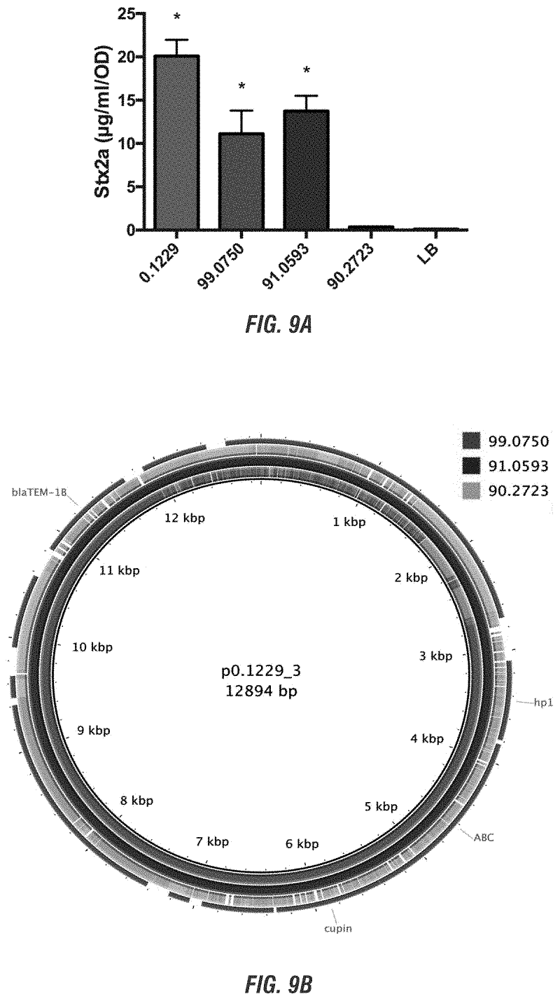

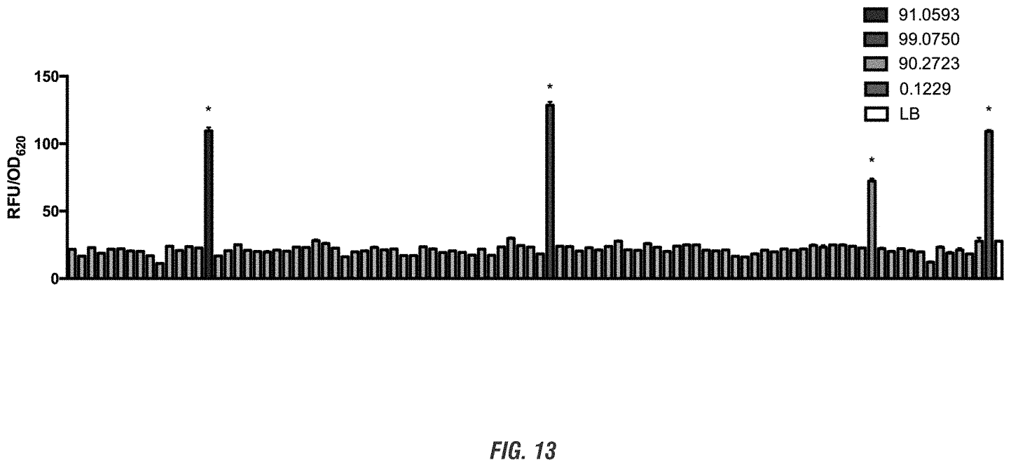

Lastly, it was hypothesized that E. coli isolated from human feces would encode the similar molecules identified here. A total of 101 human fecal E. coli isolates were obtained from Penn State's E. coli Reference Center, and three of these were found to induce GFP production in the PrecA-gfp reporter assay (FIG. 13). Furthermore, the supernatants of two of these isolates, designated 91.0593 and 99.0750, increased Stx2a to levels similar to 0.1229, however 90.2723 did not (FIG. 9A). Genome sequencing of these three organisms revealed that 91.0593 and 99.0750 carried plasmids similar to p0.1229_3, however the latter plasmid had a deletion in the recombinase and transposon regions (FIG. 9B). Strain 99.0750 was molecular serotype O36:H39, while 91.0593 could not be O typed but was identified as H10.

### Discussion

The concentration of E. coli in human feces ranges from 10⁷ to 10⁹ colony forming units (CFU) (37, 38). Typically, there are up to five commensal E. coli strains colonizing the human gut at a given time (39, 40). As the human microbiota affects O157:H7 colonization and virulence gene expression (41-44), it is thought that community differences in the gut microflora may explain, in part, individual differences in disease symptoms (45). Indeed, commensal E. coli that are susceptible to stx2-converting phage can increase phage and Stx production (22, 23). In mice given a co-culture of 0157:H7 and phage-resistant E. coli, minimal toxin was recovered in the feces, but with E. coli that were phage-susceptible, higher levels of toxin were found (46). However, it is clear that phage infection of susceptible bacteria is not the only mechanism by which the gut microflora affects Stx2 levels during infection (19, 20, 24, 26).

In this study, both whole cells and spent supernatants of E. coli 0.1229 enhanced Stx2a production by E. coli O157:H7 strain PA2. This latter strain is a member of the hypervirulent clade 8 (47) and was previously found to be a high Stx2a producer in co-culture with E. coli C600 (23). E. coli 0.1229 produces at least two molecules capable of increasing Stx2a. The first is MccB17, a DNA gyrase inhibitor, shown to activate Stx2a production in an earlier study (24). This current study identified a second molecule localized to a 12.8 kb plasmid, and all genes necessary for production are found within a 5.2 kb region. Furthermore, gene knockouts identified four potential ORFs within this region, hp1, abc, cupin and hp2, that are required for 0.1229 mediated Stx2a amplification. This gene cluster was also identified on pB51 (48), a similar plasmid to p0.1229_3, however limited characterization was reported.

Oxidizing agents, such as hydrogen peroxide (H₂O₂), and antibiotics targeting DNA replication, such as ciprofloxacin, mitomycin C and norfloxacin, are known to induce stx-converting phage (5, 49, 50) and subsequently Stx2 production (5, 49). However, the Stx2 amplifying activity of the 0.1229 supernatant was abolished by Proteinase K, suggesting the inducing molecule is proteinaceous in nature. Colicins are bacteriocins found in E. coli (51), are generally greater than 30 kDa in size, and at least one member has been previously shown to enhance O157:H7 Stx2 production (26). While some colicins utilize TonB for translocation, they are not expected to be heat stable. The molecule produced by 0.1229 was resistant to 100° C. for 10 minutes, strongly suggesting it is not a colicin.

Microcins are bacteriocins that are generally smaller than 10 kDa. Their size and lack of secondary and tertiary structure make them more heat stable than colicins. Microcins are divided into three classes; class I and class IIa are plasmid encoded, while class IIb are chromosomally encoded. Class I and IIb are post-translationally modified (52, 53), while class IIa are not. To date, all class II but only one member of class I (microcin J25) use an ATP-binding cassette (ABC)-type transporter in complex with TolC for export (35), and the TonB system for import into target cells (36). The microcin produced by 0.1229 is plasmid encoded, along with a predicted ABC transporter and is TolC and TonB dependent. Therefore, this microcin appears to be more closely related to class IIa microcins. However, purification of the microcin to identify possible post translational modifications is necessary to confirm whether designating as class I or IIa is more appropriate.

There are four known class IIa microcins, microcin V (MccV, previously named colicin V) (54, 55), microcin N (MccN, previously named Mcc24) (56), microcin L (MccL) (57), and microcin PDI (0.1229 3 containing microcin) (58, 59). The operons encoding these microcins contain four or five genes, including the microcin precursor, immunity and export genes. MccN also encodes a regulator, with a histone-like nucleoid domain (56). The microcin precursor genes possess leader sequences of approximately 15 amino acids, containing the signature sequence MRXI/LX(9)GG/A (X=any amino acid), and are typically cleaved by the ABC transporters during export (60). A potential leader sequence with the double glycine was found in hp2. Additionally, a small peptide (DHGSR) was identified in the supernatants of 0.1229 by mass spectroscopy (data not shown) corresponding to an ORF internal to hp2 encoded in the opposite direction. Future experiments will determine if one of these, or another region, encodes a secreted microcin.

One argument against designation as a class IIa microcin, is the lack of an identifiable N-terminal proteolytic domain (61) in the predicted ABC transporter encoded on p0.1229_3. This domain is found in all other members of class IIa. Interestingly, the class I microcin J25 (MccJ25) also encodes an ABC transporter lacking this domain. Unlike the other class I microcins, MccJ25 is TolC and TonB dependent for export and import, respectively. While the possibility cannot be excluded that the system identified here is a class I microcin, if so, it is more similar to MccJ25 than to other members of this group.

While the current mechanism of action is unknown, it is theorized that the microcin causes DNA damage, through double strand breaks, depurination, or inhibition of DNA replication. Such actions would lead to RecA-dependent phage induction and Stx2 production. The suspected mode of action would be divergent from the known class IIa microcins, which target the inner membrane (62) and MccJ25 which inhibits the RNA polymerase (63). Besides the predicted ABC transporter, the functions of the other ORFs is unclear. We anticipate one of these may encode an ABC accessory protein, known to be essential for these export complexes (64). One ORF encodes a cupin domain found in a functionally diverse set of proteins. An immunity gene protecting the host may also be expected in this region.

The genes encoding the microcin were additionally found in E. coli strains 99.0750 and 91.0593. Genome sequencing of these strains failed to identify genes encoding MccB17, which may explain the lower levels of Stx2a production seen in co-culture with PA2 compared to those seen with 0.1229. Bioinformatic analyses also identified other E. coli that encode nearly identical regions. Interestingly, one of these was E. coli O104:H4 HUS, isolated in 2001 (33), and responsible for a large 2011 outbreak in Germany. However, a premature stop codon identified in cupin suggests it is non-functional. Homologs of hp1, ABC and cupin were identified together in several other organisms distantly related to E. coli, suggesting these encode a functional unit. The absence of hp2 in most of these genetic clusters argues against this ORF encoding the anti-bacterial activity or may suggest that these organisms encode microcins distinct from hp2.

In conclusion, a microcin was identified in E. coli, expanding our knowledge of this small group of antimicrobial peptides. This study also identifies another mechanism by which E. coli may enhance Stx2a production by E. coli O157:H7. Further studies may also provide new insights into the diverse genetic structure and functions of microcin-encoding systems.

### Materials & Methods

### Bacterial Strains, Media and Growth Conditions

E. coli strains were grown in Lysogeny Broth (LB) at 37° C. unless otherwise indicated, and culture stocks were maintained in 20% glycerol at −80° C. Antibiotics were used at the following concentrations; ampicillin (100 μg/ml), chloramphenicol (25 μg/ml), kanamycin (50 μg/ml), and tetracycline (10 μg/ml). All bacterial isolates, plasmids and primers used in this study can be found in Table 1. E. coli SF-173-1 was provided by Dr. Craig Stephens, Santa Clara University.

Co-Culture with PA2

Co-culture with E. coli O157:H7 PA2 was performed similar to previously described (23). PA2 and commensal E. coli strains were grown overnight at 37° C. (with shaking at 250 rpm). LB agar (2.5 ml) was added to 6-well plates (BD Biosciences Inc., Franklin Lakes, N.J.), and allowed to solidify. PA2 and commensal strains were each diluted to an OD₆₀₀ of 0.05 in 1 ml of LB broth and added to the 6-well plates. A monoculture of PA2 (at 0.05 OD₆₀₀ in 1 ml) served as a negative control. The plates were incubated without shaking at 37° C. After 16 hr, cultures were collected, cells were lysed with 6 mg/ml polymyxin B at 37° C. for 5 min, and supernatants were collected. Samples were immediately tested with the receptor-based enzyme-linked immunosorbent assay (R-ELISA), as described below, or stored at −80° C. Total protein was calculated using the Bradford assay (VMR Life Science, Philadelphia, Pa.), and used to calculate μg/mg Stx2.

### R-ELISA for Stx2a Detection

Detection of Shiga toxin was performed using a sandwich ELISA approach, previously described by Xiaoli et al., 2018 (24). Briefly, 25 μg/ml of ceramide trihexosides (bottom spot) (Matreya Biosciences, Pleasant Gap, Pa.) dissolved in methanol was used for coating of the plate. Washes were performed between each step using PBS and 0.05% Tween-20. Stx2a-containing samples were diluted in PBS as necessary to obtain final readings in the linear range. Samples were added to the wells in duplicate and incubated with shaking for 1 hr at room temperature. Supernatants of E. coli PA11, a high Stx2a producer (65), were used as a positive control. Anti-Stx2 monoclonal mouse antibody (Santa Cruz Biotech, Santa Cruz Calif.) was added to the plate at a concentration of 1 μg/ml, then incubated for 1 hr. Anti-mouse secondary antibody (MilliporeSigma, Burlington Mass.) conjugated to horseradish peroxidase (1 μg/ml) was added to the plate, and incubated for 1 hr. For detection, 1 step Ultra-TMB (Thermo-Fischer, Waltham, Mass.) was used, and 2M H₂SO₄ was added to the wells to stop the reaction. The plate was read at 450 nm using a DU®730 spectrophotometer (Beckman Coulter, Atlanta, Ga.). A standard curve was generated from two-fold serially diluted PA11 samples and used to quantify the μg/ml of Stx2a present in each sample.

Cell-Free Supernatant Assay with PA2

E. coli O157:H7 strain PA2 and non-pathogenic E. coli strains were individually grown with shaking at 37° C. for 16 hr. Overnight culture of the non-pathogenic strains were centrifuged, and supernatants were filtered through 0.2 μm cellulose filters (VWR International, Radnor, Pa.). LB agar (2.5 ml) was added to the wells of 6-well plates (BD Biosciences Inc., Franklin Lakes, N.J.) and allowed to solidify. PA2 was added to wells at a final density of 0.05 OD₆₀₀ in 1 ml of spent supernatant. For the negative control, PA2 was resuspended in fresh LB broth to the same cell density, and 1 ml was added to a well. The plates were statically incubated at 37° C. for 8 hr, after which the cell density (OD₆₀₀) was recorded. Cells were lysed with 6 mg/ml Polymyxin B at 37° C. for 5 min and supernatant recovered. Samples were immediately tested for Stx2a by R-ELISA or stored at −80° C. Data reported as μg/ml/OD₆₀₀.

Detection of SOS Inducing Agents using PrecA-gfp

E. coli expressing PrecA-gfp, which encodes green fluorescent protein (gfp) under control of the recA promoter (66), was purchased from Dharmacon (Lafayette, Colo.). The plasmid was transformed into E. coli W3110ΔtolC. The tolC deletion reduces the potential efflux of recA-activating molecules. W3110ΔtolC PrecA-gfp and commensal strains were individually grown overnight with shaking at 37° C. LB agar (2.5 ml) was added to 6-well plates and allowed to solidify. W3110ΔtolC PrecA-gfp and one commensal strain were each diluted to a final OD₆₀₀ of 0.05 in LB broth, and 1 ml was added to the 6-well plates. The negative control included only W3110ΔtolC PrecA-gfp at a final OD of 0.05 in 1 ml LB broth. The plates were statically incubated at 37° C. After 16 hr, 100 μl was removed from each well, added to black 96 well clear bottom plates (Dot Scientific Inc., Burton, Mich.) and optical density (OD₆₂₀) was read using a DU®730 spectrophotometer. Relative fluorescence units (RFU) were measured at an excitation of 485 nm and emission of 538 nm on a Fluoroskan Ascent FL (Thermo Fisher Scientific, Waltham, Mass.) (67). RFU values were normalized to cell density.

One Step Recombination for E. coli Knockouts

Mutants of 0.1229 and MG1655 were constructed using one-step recombination (68). Primers contained either 50 bp upstream or downstream of the gene of interest, followed by sequences annealing to the P1 and P2 priming sites from pKD3. PCR was performed at the following settings: initial denaturation at 95° C. for 30s; 10 cycles of 95° C. 30 s, 49° C. 60 s, 68° C. 100 s; 24 cycles of 95° C. 30 s, Tₐ 60 s, 68° at variable time, and a final extension at 68° C. for 5 min. Tₐ and variable times for each set of primers are reported in Table 1. A derivative of pKD46-Kan^{g} was used as 0.1229 is resistant to Amp^{R}. Electroporation was used to construct E. coli 0.1229(pKD46) and MG1655(pKD46), using a Bio-Rad Gene Pulser II and following protocols recommended by the manufacturer. Colonies containing pKD46-Kan^{R} were selected on LB plates with kanamycin. Strains containing pKD46 were grown to an OD₆₀₀ of 0.3, and L-arabinose was added to a final concentration of 0.2M. After incubation for 1 hr, cells were washed and electroporated with the pKD3-derived PCR product. Transformants were selected on LB plates with chloramphenicol. Knockouts were confirmed by PCR using primers ˜200 bp upstream and downstream of the gene, using standard PCR settings (initial denaturation at 95° C. for 30 s; 35 cycles of 95° C. 30 s, variable amplification temperature (Tₐ) 60 s, 68° C. at variable time; and a final extension at 68° C. for 5 min). This strategy was followed for all the knockouts, including primers and temperatures specific for each gene (Table 1).

### Gibson Cloning

The 2745-7950 bp region of p0.1229_3 was cloned into pBR322 (pBR322:: p0.1229_3²⁷⁴⁵⁻⁷⁹⁵⁰), using Gibson cloning as previously described (69). Briefly, primer pairs were constructed containing 30 bp annealing to the pBR322 insert site and 30 bp that would anneal to p0.1229_3. DNA from 0.1229 and pBR322 was amplified at these sites using standard PCR settings, amplicons were cleaned up using a PCR purification kit (Qiagen, Germantown, Md.) and subjected to assembly at 50° C. using the Gibson cloning kit (New England Biosciences, Ipswich, Mass.). Assembled plasmids were propagated in DH5α competent cells (New England Biosciences, Ipswich, Mass.). Verification PCR was performed using primers 200 bp upstream and downstream of the insert site (Table 1) and confirmed using Sanger sequencing. Successful constructs were transformed into C600 electrocompetent cells. A similar process was used to clone tolC in pBAD18 (Kan^{R}).

### Whole Genome Sequencing and Bioinformatics

For the whole genome sequencing of 0.1229, genomic DNA was isolated using the Wizard Genomic DNA purification kit (Promega, Madison, Wis.). Whole genome sequencing was performed at the Penn State Genomics Core facility using the Illumina MiSeq platform. A PCR-free DNA kit was used for library preparation. The sequencing run produced 2×150 bp reads.

For the whole genome sequencing of 99.0750, 91.0593, and 90.2723, genomic DNA was isolated using Qiagen DNeasy Blood and Tissue Kit (Qiagen Inc., Germantown, Md.). Whole genome sequencing was performed using the NexTera XT DNA library prep kit and run on an Illumina MiSeq platform. The sequencing run produced 2×250 bp reads.

After Illumina sequencing, Fastq files were checked using Fastqc v0.11.5 (70) and assembled using SPAdes v3.10 (71). SPAdes assemblies were subjected to the Quality Assessment Tool for Genome Assemblies v4.5 (QUAST) (72), and contig number, genome size, N50 and GC % were noted.

Strain 0.1229 was also sequenced at the Center for Food Safety and Nutrition, Food and Drug Administration using the Pacific Biosciences (PacBio) RS II sequencing platform, as previously reported (73). For library preparation, 10 μg genomic DNA was sheared to 20 kb fragments by g-tubes (Covaris, Inc., Woburn, Mass., USA) according to the manufacturer's instructions. The SMRTbell 20 kb template library was constructed using DNA Template Prep kit 1.0 (Pacific Biosciences, Menlo Park, Calif., USA). BluePippin (Sage Science, Beverly, Mass., USA) was used for size selection, and sequencing was performed using the P6/C4 chemistry on two single-molecule real-time (SMRT) cells with a 240 min collection protocol along with stage start. SMRT Analysis 2.3.0 was used for read analysis, and de novo assembly using the PacBio Hierarchical Genome Assembly Process (HGAP3.0) program. The assembly output from HGAP contained overlapping regions at the end which can be identified using dot plots in Gepard (74). The genome was checked manually for even sequencing coverage. Afterwards, the improved consensus sequence was uploaded in SMRT Analysis 2.3.0 to determine the final consensus and accuracy scores using Quiver consensus algorithm (75). The assembled genome was annotated using the NCBI's Prokaryotic Genomes Automatic Annotation Pipeline (PGAAP) (76).

Plasmid sequences were visualized using Blast Ring Image Generator v0.95 (BRIG) (77). The Center for Genomic Epidemiology website was used for ResFinder v3.1.0 (90% identity, 60% length) (78), SerotypeFinder v2.0.1 (85% identity, 60% length) (79) and MLSTFinder v2.0.1 (80) using the Achtman multi-locus sequence typing (MLST) scheme (81). The Integrated Microbial Genomics & Microbiomes website of DOE's Joint Genome Institute was utilized to BLAST the amino acid sequence of Hp1 against other genomes, matches that were between 36 and 68% identical from varying species were selected, then visualized using the gene neighborhoods function (82).

### Data Analysis

MS Excel (Microsoft Corporation, Albuquerque N. Mex.) was used to calculate the mean, standard deviation, and standard error; and GraphPad Prism 6 (GraphPad Software, San Diego Calif.) was used for generating figures. Error bars report standard error of the mean from at least three biological replicates.

### Data Availability

Nucleotide and SRA files for the 0.1229 can be found on NCBI under Biosample SAMN08737532. SRA files for 99.0750 (SAMN11457477), 91.0593 (SAMN11457478), 90.2723 (SAMN11457479) can be found under their respective accession numbers.

### REFERENCES

1. Griffin PM, Tauxe R V. 1991. The Epidemiology of Infections Caused by Escherichia coli O157:H7, Other Enterohemorrhagic E. coli, and the Associated Hemolytic Uremic Syndrome. Epidemiol Rev 13:60-98.
2. Nguyen Y, Sperandio V, Padola N L, Starai V J. 2012. Enterohemorrhagic E. coli (EHEC) pathogenesis. Front Cell Infect Microbiol 2:1-7.
3. Hayashi T, Makino K, Ohnishi M, Kurokawa K, Ishii K, Yokoyama K, Han C-G, Ohtsubo E, Nakayama K, Murata T, Tanaka M, Tobe T, lida T, Takami H, Honda T, Sasakawa C, Ogasawara N, Yasunaga T, Kuhara S, Shiba T, Hattori M, Shinagawa H. 2001. Complete Genome Sequence of Enterohemorrhagic Eschelichia coli O157:H7 and Genomic Comparison with a Laboratory Strain K-12. DNA Res 8:11-22.
4. Waldor M K, Friedman D I. 2005. Phage Regulatory Circuits and Virulence Gene Expression. Curr Opin Microbiol 8:459-465.
5. Zhang X, McDaniel A D, Wolf L E, Keusch G T, Waldor M K, Acheson D W. 2000. Quinolone Antibiotics induce Shiga toxin-encoding Bacteriophages, Toxin production, and Death in Mice. J Infect Dis 181:664-670.
6. Scotland S, Smith H R, Rowe B. 1985. Two Distinct Toxins active on Vero cells from Escherichia coli O157. Lancet 2:885-886.
7. Scheutz F, Teel L D, Beutin L, Piérard D, Buvens G, Karch H, Mellmann A, Caprioli A, Tozzoli R, Morabito S, Strockbine N A, Melton-Celsa AR, Sanchez M, Persson S, O'Brien A D. 2012. Multicenter Evaluation of a Sequence-based Protocol for Subtyping Shiga toxins and Standardizing Stx Nomenclature. J Clin Microbiol 50:2951-2963.
8. Bai X, Fu S, Zhang J, Fan R, Xu Y, Sun H, He X, Xu J, Xiong Y. 2018. Identification and Pathogenomic Analysis of an Escherichia coli Strain Producing a Novel Shiga toxin 2 Subtype. Sci Rep 8:1-11.
9. FAO/WHO STEC Expert Group. 2018. Hazard Identification and Characterization: Criteria for Categorizing Shiga Toxin-Producing Escherichia coli on a Risk Basis. J Food Prot 82:7-21.
10. Strockbine N A, Marques L R, Newland J W, Smith H W, Holmes R K, O'Brien A D. 1986. Two toxin-converting Phages from Escherichia coli O157:H7 strain 933 encode Antigenically Distinct Toxins with Similar Biologic Activities. Infect Immun 53:135-140.
11. Matsushiro A, Sato K, Miyamoto H, Yamamura T, Honda T. 1999. Induction of Prophages of Enterohemorrhagic Escherichia coli O157:H7 with Norfloxacin. J Bacteriol 181:2257-2260.
12. Ostroff S, Tarr P, Neill M A, Lewi J H, Hargrett-Bean N, Ostroff S, Tarr P, Neill M A, Lewi J H, Kobayashi J M. 1989. Toxin Genotypes and Plasmid Profiles as Determinants of Systemic Sequelae in Escherichia coli O157:H7 Infections. J Infect Dis 160:994-998.
13. Donohue-Rolfe A, Kondova I, Oswald S, Hutto D, Tzipori S. 2000. Escherichia coli O157:H7 Strains That Express Shiga Toxin (Stx) 2 Alone Are More Neurotropic for Gnotobiotic Piglets Than Are Isotypes Producing Only Stx1 or Both Stx1 and Stx2. J Infect Dis 181:1825-1829.
14. Orth D, Grif K, Khan A B, Naim A, Dierich M P, Würzner R. 2007. The Shiga toxin Genotype Rather than the Amount of Shiga toxin or the Cytotoxicity of Shiga toxin in vitro Correlates with the Appearance of the Hemolytic Uremic Syndrome. Diagn Microbiol Infect Dis 59:235-242.
15. Persson S, Olsen K E P, Ethelberg S, Scheutz F. 2007. Subtyping Method for Escherichia coli Shiga Toxin (Verocytotoxin) 2 Variants and Correlations to Clinical Manifestations. J Clin Microbiol 45:2020-2024.
16. Shringi S, Schmidt C, Katherine K, Brayton K A, Hancock D D, Besser T E. 2012. Carriage of stx2a Differentiates Clinical and Bovine-Biased Strains of Escherichia coli O157. PLoS One 7:e51572.
17. Kawano K, Okada M, Haga T, Maeda K, Goto Y. 2008. Relationship between Pathogenicity for Humans and stx Genotype in Shiga toxin-Producing Escherichia coli Serotype O157. Eur J Clin Microbiol Infect Dis 27:227-232.
18. FAO/WHO. 2018. Shiga toxin-producing Escherichia coli (STEC) and Food: Attribution, Characterization, and Monitoring. Rome.
19. Carey C M, Kostrzynska M, Ojha S, Thompson S. 2008. The Effect of Probiotics and Organic Acids on Shiga-toxin 2 Gene Expression in Enterohemorrhagic Escherichia coli O157:H7. J Microbiol Methods 73:125-132.
20. Thévenot J, Cordonnier C, Rougeron A, Le Goff O, Nguyen H T T, Denis S, Alric M, Livrelli V, Blanquet-Diot S. 2015. Enterohemorrhagic Escherichia coli Infection has Donor-dependent Effect on Human Gut Microbiota and May be Antagonized by Probiotic Yeast during Interaction with Peyer's Patches. Appl Microb Cell Physiol 99:9097-9110.
21. de Sablet T, Chassard C, Bernalier-Donadille A, Vareille M, Gobert A P, Martin C. 2009. Human Microbiota-Secreted Factors Inhibit Shiga Toxin synthesis by Enterohemorrhagic Escherichia coli O157:H7. Infect Immun 77:783-790.
22. Gamage S D, Strasser J E, Chalk C L, Weiss A A. 2003. Nonpathogenic Escherichia coli Can Contribute to the Production of Shiga Toxin. Infect Immun 71:3107-3115.
23. Goswami K, Chen C, Xiaoli L, Eaton K A, Dudley E G. 2015. Coculture of Escherichia coli O157:H7 with a Nonpathogenic E . coli Strain Increases Toxin Production and Virulence in a Germfree Mouse Model. Infect Immun 83:4185-4193.
24. Xiaoli L, Figler H M, Goswami K, Dudley E G. 2018. Nonpathogenic E. coli Enhance Stx2a Production of E. coli O157:H7 through bamA-Dependent and Independent Mechanisms. Front Microbiol 9:1-13.
25. Smith D L, James C E, Sergeant M J, Yaxian Y, Saunders J R, McCarthy A J, Allison H E. 2007. Short-tailed stx phages Exploit the Conserved YaeT Protein to Disseminate Shiga Toxin Genes Among Enterobacteria. J Bacteriol 189:7223-7233.
26. Toshima H, Yoshimura A, Arikawa K, Hidaka A, Ogasawara J, Hase A, Masaki H, Nishikawa Y. 2007. Enhancement of Shiga Toxin Production in Enterohemorrhagic Escherichia coli Serotype O157:H7 by DNase Colicins. Appl Environ Microbiol 73:7582-7588.
27. Mobley H L T, Green D M, Trifillis A L, Johnson D E, Chippendale G R, Lockatell C V, Jones B D, Warren J W. 1990. Pyelonephritogenic Escherichia coli and Killing of Cultured Human Renal Proximal Tubular Epithelial Cells: Role of Hemolysin in Some Strains. Infect Immun 58:1281-1289.
28. Nissle A. 1919. Weiteres über die Mutaflorbehandlung unter besonderer Berü cksichtigung der chronischen Ruhr. Münchener Medizinische Wochenschrift 25:678-681.
29. Wijetunge D S S, Karunathilake K H E M, Chaudhari A, Katani R, Dudley E G, Kapur V, DebRoy C, Kariyawasam S. 2014. Complete Nucleotide Sequence of pRS218, a Large Virulence Plasmid, that Augments Pathogenic Potential of Meningitis-associated Escherichia coli Strain RS218. BMC Microbiol 14:1-16.
30. Chen S L, Hung C-S, Xu J, Reigstad C S, Magrini V, Sabo A, Blasiar D, Bieri T, Meye R R, Ozersky P, Armstrong J R, Fulton R S, Latreille J P, Spieth J, Hooton T M, Mardis E R, Hultgren S J, Gordon J I. 2006. Identification of Genes Subject to Positive Selection in Uropathogenic Strains of Escherichia coli: A Comparative Genomics Approach. PNAS 103:5977-5982.
31. Davagnino J, Herrero M, Furlong D, Moreno F, Kolter R. 1986. The DNA Replication Inhibitor Microcin B17 is a Forty-three-amino-acid Protein Containing Sixty Percent Glycine. Proteins Struct Funct Genet 1:230-238.
32. Stephens C M, Skerker C M, Sekhon J M, Arkin M S, Riley A P. 2015. Complete Genome Sequences of Four Escherichia coli ST95 Isolates from Bloodstream Infections. Genome Announc 3:1241-1256.
33. Künne C, Billion A, Mshana S E, Schmiedel J, Domann E, Hossain H, Hain T, Imirzalioglu C, Chakraborty T. 2011. Complete Sequences of Plasmids from the Hemolytic-uremic Syndrome-associated Escherichia coli strain HUSEC41. J Bacteriol 194:532-533.
34. Thompson R E, Collin F, Maxwell A, Jolliffe K A, Payne R J. 2014. Synthesis of Full Length and Truncated Microcin B17 Analogues as DNA Gyrase Poisons. Org Biomol Chem 12:1570-1578.
35. Delgado M A, Solbiati J O, Chiuchiolo M J, Farias R N, Salomon R A. 1999. Escherichia coli Outer Membrane Protein TolC is Involved in Production of the Peptide Antibiotic Microcin J25. J Bacteriol 181:1968-1970.
36. Braun V, Patzer S I, Hantke K. 2002. Ton-dependent Colicins and Microcins: Modular Design and Evolution. Biochimie 84:365-380.
37. Penders J, Thijs C, Vink C, Stelma F F, Snijders B, Kummeling I, van den Brandt P A, Stobberingh E E. 2006. Factors Influencing the Composition of the Intestinal Microbiota in Early Infancy. Pediatrics 118:511-521.
38. Slanetz L W, Bartley C H. 1957. Numbers of Enterococci in Water, Sewage, and Feces determined by the Membrane Filter Technique with an improved medium. J Bacteriol 74:591-595.
39. Apperloo-renkema H Z, Van Der Waaij B D, Van Der Waaij D. 1990. Determination of Colonization Resistance of the Digestive Tract by Biotyping of Enterobacteriaceae. Epidemiol Infect 105:355-361.
40. Johnson J R, Owens K, Gajewski A, Clabots C. 2008. Escherichia coli Colonization Patterns among Human Household Members and Pets, with Attention to Acute Urinary Tract Infection. J Infect Dis 197:218-224.
41. Leatham M P, Banerjee S, Autieri S M, Conway T, Cohen P S, Mercado-lubo R. 2009. Precolonized Human Commensal Escherichia coli Strains Serve as a Barrier to E. coli O157: H7 Growth in the Streptomycin-Treated Mouse Intestine. Infect Immun 77:2876-2886.
42. Sperandio V, Mellies J L, Nguyen W, Shin S, Kaper J B. 1999. Quorum Sensing Controls Expression of the Type III Secretion Gene Transcription and Protein Secretion in Enterohemorrhagic and Enteropathogenic Escherichia coli. Proc Natl Acad Sci USA 96:15196-15201.
43. Sperandio V, Tones A G, Gir N J A, Kaper J B. 2001. Quorum Sensing Is a Global Regulatory Mechanism in Enterohemorrhagic Escherichia coli O157:H7. J Bacteriol 183:5187-5197.
44. Zhao T, Doyle M P, Harmon B G, Brown C A, Mueller P O, Parks A H. 1998. Reduction of Carriage of Enterohemorrhagic Escherichia coli O157:H7 in Cattle by Inoculation with Probiotic Bacteria. J Clin Microbiol 36:641-647.
45. Bell B P, Griffin P M, Lozano P, Christie D L, Kobayashi J M, Tarr P I. 1997. Predictors of Hemolytic Uremic Syndrome in Children During a Large Outbreak of Escherichia coli O157:H7 Infections. Pediatrics 100:1-6.
46. Gamage S D, Patton A K, Strasser J E, Chalk C L, Weiss A A. 2006. Commensal Bacteria Influence Escherichia coli O157:H7 Persistence and Shiga toxin Production in the Mouse Intestine. Infect Immun 74:1977-1983.
47. Amigo N, Mercado E, Bentancor A, Singh P, Vilte D, Gerhardt E, Zotta E, Ibarra C, Manning S D, Larzábal M, Cataldi A. 2015. Clade 8 and Clade 6 Strains of Escherichia coli O157:H7 from Cattle in Argentina have Hypervirulent-Like Phenotypes. PLoS One 10:e0127710.
48. Micenková L. 2016. PhD Thesis. Bacteriocinogeny in pathogenic and commensal Escherichia coli strains. Masarykova Univerzita Léka\check{u}ská Fakulta Biologický Ú stay.
49. Bielaszewska M, Idelevich E A, Zhang W, Bauwens A, Schaumburg F, Mellmann A, Peters G, Karch H. 2012. Effects of Antibiotics on Shiga toxin 2 Production and Bacteriophage Induction by Epidemic Escherichia coli O104:H4 Strain. Antimicrob Agents Chemother 56:3277-3282.
50. Łoś J M, Łoś M, Węgrzyn A, Wegrzyn G. 2010. Hydrogen Peroxide-mediated Induction of the Shiga Toxin Converting Lambdoid Prophage ST2-8624 in Escherichia coli O157:H7. FEMS Immunol Med Microbiol 58:322-329.
51. Cascales E, Buchanan S K, Duche D, Kleanthous C, Lloubes R, Postle K, Riley M, Slatin S, Cavard D. 2007. Colicin Biology. Microbiol Mol Biol Rev 71:158-229.
52. Duquesne S, Destoumieux-Garzón D, Peduzzi J, Rebuffat S. 2007. Microcins, Gene-encoded Antibacterial Peptides from Enterobacteria. R Soc Chem 24:708-734.
53. Patzer S I, Baquero M R, Bravo D, Moreno F, Hantke K. 2003. The Colicin G, H and X Determinants Encode Microcins M and H47, which Might Utilize the Catecholate Siderophore Receptors FepA, Cir, Fiu and IroN. Microbiology 149:2557-2570.
54. Gilson L, Mahanty K, Kolter R. 1987. Four Plasmid Genes Are Required for Colicin V Synthesis, Export, and Immunity. J Bacteriol 169:2466-2470.
55. Chehade H, Braun V. 1988. Iron-regulated Synthesis and Uptake of Colicin V. FEMS Microbiol Lett 52:177-181.
56. Corsini G, Karahanian E, Tello M, Fernandez K, Rivero D, Saavedra J M, Ferrer A. 2010. Purification and Characterization of the Antimicrobial Peptide Microcin N. FEMS Microbiol Lett 312:119-125.
57. Morin N, Lanneluc I, Connil N, Cottenceau M, Pons A M, Sablé S. 2011. Mechanism of Bactericidal Activity of Microcin L in Escherichia coli and Salmonella enterica. Antimicrob Agents Chemother 55:997-1007.
58. Eberhart L J, Deringer J R, Brayton K a., Sawant A a., Besser T E, Call DR. 2012. Characterization of a Novel Microcin that Kills Enterohemorrhagic Escherichia coli O157:H7 and O26. Appl Environ Microbiol 78:6592-6599.
59. Zhao Z, Orfe L H, Liu J, Lu S-Y, Besser T E, Call D R. 2017. Microcin PDI Regulation and Proteolytic Cleavage are Unique Among Known Microcins. Nat Publ Gr 7:1-14.
60. Havarstein L S, Holo H, Nes I F. 1994. The Leader Peptide of Colicin V Shares Consensus Sequences with Leader Peptides that are Common Among Peptide Bacteriocins Produced by Gram-positive Bacteria. Microbiology 140:2383-2389.
61. Havarstein L S, Diep D B, Nes I F. 1995. A Family of Bacteriocin ABC transporters Carry out Proteolytic Processing of their Substrates Concomitant with Export. Mol Microbiol 16:229-240.
62. Yang C C, Konisky J. 1984. Colicin V-treated Escherichia coli Does Not Generate Membrane Potential. J Bacteriol 158:757-759.
63. Yuzenkova J, Delgado M, Nechaev S, Savalia D, Epshtein V, Artsimovitch I, Mooney R A, Landick R, Farias R N, Salomon R, Severinov K. 2002. Mutations of Bacterial RNA Polymerase Leading to Resistance to Microcin J25. J Biol Chem 277:50867-50875.
64. Gilson L, Mahanty H K, Kolter R. 1990. Genetic Analysis of an MDR-like Export System: The Secretion of Colicin V. EMBO J 9:3875-3884.
65. Hartzell A, Chen C, Lewis C, Liu K, Reynolds S, Dudley EG. 2011. Escherichia coli O157:H7 of Genotype Lineage-Specific Polymorphism Assay 211111 and Clade 8 Are Common Clinical Isolates Within Pennsylvania. Foodborne Pathog Dis 8:763-768.
66. Zaslaver A, Bren A, Ronen M, Itzkovitz S, Kikoin I, Shavit S, Liebermeister W, Surette M G, Alon U. 2006. A Comprehensive Library of Fluorescent Transcriptional Reporters for Escherichia coli. Nat Methods 3:623-628.
67. Fan J, de Jonge B L M, MacCormack K, Sriram S, McLaughlin R E, Plant H, Preston M, Fleming P R, Albert R, Foulk M, Mills S D. 2014. A Novel High-throughput Cell-based Assay Aimed at Identifying Inhibitors of DNA Metabolism in Bacteria. Antimicrob Agents Chemother 58:7264-7272.
68. Datsenko K, Wanner B L. 2000. One-step inactivation of chromosomal genes in Escherichia coli K-12 using PCR products. Proc Natl Acad Sci USA 97:6640-6645.
69. Gibson D G, Young L, Chuang R-Y, Venter J C, Hutchison C A, Smith H O. 2009. Enzymatic Assembly of DNA Molecules up to Several Hundred Kilobases. Nat Methods 6:343-345.
70. Andrews S. 2010. FastQC: A Quality Control Tool for High Throughput Sequence Data. Babraham Bioinformatics. https://www.bioinformatics.babraham.ac.uk/projects/fastqc/
71. Bankevich A, Nurk S, Antipov D, Gurevich A A, Dvorkin M, Kulikov A S, Lesin V M, Nikolenko S I, Pham S, Prjibelski A D, Pyshkin A V., Sirotkin A V., Vyahhi N, Tesler G, Alekseyev M A, Pevzner P A. 2012. SPAdes: A New Genome Assembly Algorithm and Its Applications to Single-Cell Sequencing. J Comput Biol 19:455-477.
72. Gurevich A, Saveliev V, Vyahhi N, Tesler G. 2013. QUAST: Quality Assessment Tool for Genome Assemblies. Bioinformatics 29:1072-1075.
73. Yao K, Roberts R J, Allard M W, Hoffmann M. 2017. Complete Genome and Methylome Sequences of Salmonella enterica subsp. enterica Serovars Typhimurium, Saintpaul, and Stanleyville from the SARA/SARB Collection. Genome Announc 5:e00031-17.
74. Krumsiek J, Arnold R, Rattei T. 2007. Gepard: A Rapid and Sensitive Tool for Creating Dotplots on Genome Scale. Bioinformatics 23:1026-1028.
75. Chin C-S, Alexander D H, Marks P, Klammer A A, Drake J, Heiner C, Clum A, Copeland A, Huddleston J, Eichler E E, Turner S W, Korlach J. 2013. Nonhybrid, Finished Microbial Genome Assemblies from Long-read SMRT Sequencing Data. Nat Methods 10:563-569.
76. Klimke W, Agarwala R, Badretdin A, Chetvernin S, Ciufo S, Fedorov B, Kiryutin B, O'Neill K, Resch W, Resenchuk S, Schafer S, Tolstoy I, Tatusova T. 2009. The National Center for Biotechnology Information's Protein Clusters Database. Nucleic Acids Res 37:D216-D223.
77. Alikhan N-F, Petty N K, Ben Zakour N L, Beatson S A. 2011. BLAST Ring Image Generator (BRIG): Simple Prokaryote Genome Comparisons. BMC Genomics 12:1-10.
78. Zankari E, Hasman H, Cosentino S, Vestergaard M, Rasmussen S, Lund O, Aarestrup F M, Larsen M V. 2012. Identification of Acquired Antimicrobial Resistance Genes. J Antimicrob Chemother 67:2640-2644.
79. Joensen K G, Tetzschner A M M, Iguchi A, Aarestrup F M, Scheutz F. 2015. Rapid and Easy In Silico Serotyping of Escherichia coli Isolates by Use of Whole-Genome Sequencing Data. J Clin Microbiol 53:2410-2426.
80. Larsen M V, Cosentino S, Rasmussen S, Friis C, Hasman H, Marvig R L, Jelsbak L, Sicheritz-Pontén T, Ussery D W, Aarestrup F M, Lund O. 2012. Multilocus Sequence Typing of Total-genome-sequenced Bacteria. J Clin Microbiol 50:1355-1361.
81. Wirth T, Falush D, Lan R, Colles F, Mensa P, Wieler L H, Karch H, Reeves P R, Maiden M C J, Ochman H, Achtman M. 2006. Sex and Virulence in Escherichia coli: An Evolutionary Perspective. Mol Microbiol 60:1136-1151.
82. Chen I-MA, Chu K, Palaniappan K, Pillay M, Ratner A, Huang J, Huntemann M, Varghese N, White JR, Seshadri R, Smirnova T, Kirton E, Jungbluth S P, Woyke T, Eloe-Fadrosh E A, Ivanova N N, Kyrpides N C. 2019. IMG/M v.5.0: An Integrated Data Management and Comparative Analysis System for Microbial Genomes and Microbiomes. Nucleic Acids Res 47:D666-D677.
83. Riley L W, Remis R S, Helgerson S D, McGee H B, Wells J G, Davis B R, Hebert R J, Olcott E S, Johnson L M, Hargrett N T, Blake P A, Cohen M L. 1983. Hemorrhagic Colitis Associated with a Rare Escherichia coli Serotype. N Engl J Med 308:681-685.
84. Appleyard R K. 1954. Segregation of New Lysogenic Types During Growth of a Doubly Lysogenic Strain Derived from Escherichia coli K12. Genetics 39:440-452.
85. Blattner F R, Plunkett G, Bloch C A, Perna N T, Burland V, Riley M, Collado-Vides J, Glasner J D, Rode C K, Mayhew G F, Gregor J, Davis N W, Kirkpatrick H A, Goeden M A, Rose D J, Mau B, Shao Y. 1997. The complete genome sequence of Escherichia coli K-12. Science (80) 277:1453-1462.
86. Achtman M, Mercer A, Kusecek B, Pohl A, Heuzenroeder M, Aaronson W, Sutton A, Silver R P. 1983. Six widespread bacterial clones among Escherichia coli K1 isolates. Infect Immun 39:315-35.
87. Yorgey P, Lee J, Kordel J, Vivas E, Warner P, Jebaratnam D, Kolter R. 1994. Posttranslational modifications in microcin B17 define an additional class of DNA gyrase inhibitor. Proc Natl Acad Sci USA 91:4519-4523.
88. Guzman L-M, Belin D, Carson M J, Beckwith J. 1995. Tight Regulation, Modulation, and High-Level Expression by Vectors Containing the Arabinose pBAD Promoter. J Bacteriol. 177(14):4121-4130.
89. Larsen R A, Thomas M G, Postle K. 1999. Protonmotive force, ExbB and ligand-bound FepA drive conformational changes in TonB. Mol Microbiol 31:1809-1824.
90. Bolivar F, Rodriguez R L, Greene P J, Betlach M C, Heyneker H L, Boyer H W, Crosa J H, Falkow S. 1977. Construction and characterization of new cloning vehicle. II. A multipurpose cloning system. Gene 2:95-113.

TABLE 1
Bacterial isolates, plasmids and primers used in this study

| E. coli strains | Characteristic(s) | Reference |
|---|---|---|
| PA2 | stx2a; O157:H7; Pennsylvania | (65) |
| PA8 | stx2a; O157:H7; Pennsylvania | (65) |
| EDL933 | stx2a, stx1a; O157:H7 | (83) |
| C600 | K12 derivative | (84) |
| MG1655 | K12 derivative | (85) |
| 1.0484 | A phylogroup; O147; Minnesota | ECRC |
| 0.1229 | B2 phylogroup; O18:H1; AmpR TetR; ST73; California | ECRC |
| 1.0342 | D phylogroup; O11; Minnesota | ECRC |
| 1.1967 | B2 phylogroup; O21; Minnesota | ECRC |
| 1.0374 | D phylogroup; O77; Minnesota | ECRC |
| Nissle 1917 | Mutaflor; O6:H1; ST73 | (28) |
| CFT073 | UPEC; O6:H1; ST73 | (27) |
| RS218 | NMEC; O18:H7; ST95 | (86) |
| 99.0750 | 036:H39; Brazil | ECRC |
| 91.0593 | O?:H10; Mexico | ECRC |
| 90.2723 | O?:H12; New York | ECRC |
| ZK1526 | microcin B17 producing strain; W3110 ΔlacU169 tna-2 pPY113; Amp^{R} | (87) |

| Derivatives | Characteristic(s) | Antibiotic resistance | Reference |
|---|---|---|---|
| 0.1229ΔmcbA | 0.1229ΔmcbA::cat | Cat^{R} Amp^{R} Tet^{R} | This study |
| 0.122ΔmcbABCDEFG | 0.1229ΔmcbABCDEFG::cat | Cat^{R} Amp^{R} Tet^{R} | This study |
| 0.1229Δ6 | 0.1229Δp0.1229_3²⁸⁵⁰⁻⁵⁴⁷³::Cat | Cat^{R} Amp^{R} Tet^{R} | This study |
| 0.1229Δ7 | 0.1229Δp0.1229_3⁵⁴²⁶⁻⁷⁹⁵⁰::Cat | Cat^{R} Amp^{R} Tet^{R} | This study |
| 0.1229Δ8 | 0.1229Δp0.1229_3⁸⁰⁰¹⁻⁹⁹⁵⁰::cat | Cat^{R} Amp^{R} Tet^{R} | This study |
| 0.1229Δhp1 | 0.1229Δp0.1229_3³⁰⁸⁴⁻³⁷⁹²::Cat | Cat^{R} Amp^{R} Tet^{R} | This study |
| 0.1229ΔABC | 0.1229Δp0.1229_3³⁸³¹⁻⁵⁴²³::Cat | Cat^{R} Amp^{R} Tet^{R} | This study |
| 0.1229Δcupin | 0.1229Δp0.1229_3⁵⁴²⁶⁻⁶³¹⁹::cat | Cat^{R} Amp^{R} Tet^{R} | This study |
| 0.1229ΔDUF4440 | 0.1229Δp0.1229_3⁶⁷⁰⁶⁻⁶³⁴⁴::cat | Cat^{R} Amp^{R} Tet^{R} | This study |
| 0.1229ΔDUF2164 | 0.1229Δp0.1229_3⁶⁹⁴²⁻⁶⁷⁰³::cat | Cat^{R} Amp^{R} Tet^{R} | This study |
| 0.1229Δhp2 | 0.1229Δp0.1229_3⁷²²⁷⁻⁷⁰⁴⁸::cat | Cat^{R} Amp^{R} Tet^{R} | This study |
| 0.1229Δhp3 | 0.1229Δp0.1229_3⁹⁰⁹⁹⁻⁷⁵⁴⁶::cat | Cat^{R} Amp^{R} Tet^{R} | This study |
| 0.1229ΔtolC | 0.1229ΔtolC::cat | Cat^{R} Amp^{R} Tet^{R} | This study |
| 0.1229ΔtolC pBAD18:: | 0.1229ΔtolC::cat + pBAD18::tolC | Cat^{R} Amp^{R} Tet^{R} Kan^{R} | This study |
| tolC | | | |
| 0.1229ΔtolC pBAD18 | 0.1229ΔtolC::cat + pBAD18 | Cat^{R} Amp^{R} Tet^{R} Kan^{R} | This study |
| W3110ΔtolC PrecA-GFP | W3110ΔtolC::tet + PrecA-GFP | Tet^{R} Kan^{R} | (66) |
| MG1655 PrecA-GFP | MG1655 PrecA-GFP | Kan^{R} | This study |
| MG1655ΔtonB PrecA-GFP | MG1655ΔtonB::cat + PrecA-GFP | Cat^{R} Kan^{R} | This study |
| MG1655ΔtonB PrecA-GFP | MG1655ΔtonB::cat + PrecA-GFP + pKP215 | Cat^{R} Kan^{R} Amp^{R} | This study |
| pKP315 | | | |
| MG1655ΔtonB PrecA-GFP | MG1655ΔtonB::cat + PrecA-GFP + pBAD24 | Cat^{R} Kan^{R} Amp^{R} | This study |
| pBAD24 | | | |
| C600 pBR322:: | C600 + pBR322::p0.1229_3²⁷⁴⁵⁻⁷⁹⁵⁰ | Tet^{R} | This study |
| p0.1229_³²⁷⁴⁵⁻⁷⁹⁵⁰ | | | |
| C600 pBR322 | C600 + pBR322 | Amp^{R} Tet^{R} | This study |
| C600 p0.1229_3 | C600 + p0.1229_3 | Amp^{R} | This study |
| Plasmids | Characteristic(s) | Antibiotic resistance | Reference |
| p0.1229_1 | 114 kb plasmid of 0.1229 | None | This study |
| p0.1229_2 | 96 kb plasmid of 0.1229 | Tet^{R} | This study |
| p0.1229_3 | 13 kb plasmid of 0.1229 | Amp^{R} | This study |
| pKD3 | pKD3 | Cat^{R}, Amp^{R} | (68) |
| pKD46-Kan^{R} | pKD46; pCRISPR, P_{araC}-λ,red recombinase | Kan^{R} | Nikki Shariat |
| PrecA-GFP | pMSs201 + PrecA-GFP | Kan^{R} | (66) |
| pBAD24 | pBAD24; araC | Amp^{R} | (88) |
| pKP315 | pBAD24::tonB; P_{araC} | Amp^{R} | (89) |
| pBAD18 | pBAD18; P_{araC} | Kan^{R} | (88) |
| pBAD18::tolC | pBAD18::tolC; P_{araC} | Kan^{R} | This study |
| pBR322 | pBR322 | Amp^{R}, Tet^{R} | (90) |
| pBR322::hplend7 | pBR322::p0.1229_3²⁸⁵⁰⁻⁷⁹⁵⁰ | Tet^{R} | This study |

| Primers | Primer | | Ta, variable |
|---|---|---|---|
| Experiment | Name | Sequence | time |
| ΔmcbA & | mcbA-KF | atactattcagatgtcataagcattaatttcccttaaaaaaggagtccttGTGTAGGCTGGAGC | 62° C., 100s |
| ΔmcbABCDEFG | | TGCTTC SEQ ID NO: 17 | |
| | mcbA-KR | ttattaatatcagggagcaccatgctccctgaacggttaattcaacgtaCATATGAATATCCT | |
| | | CCTTAG SEQ ID NO: 18 | |
| ΔmcbA | mcbA-VF | GGGGCTTAAAGGGGTAGTGT SEQ ID NO: 19 | 49° C., 45s |
| | mcbA-VR | AAGCGATTCGTCCAGTAGTTT SEQ ID NO: 20 | |
| ΔmcbABCDEFG | mcbG-KR | gtccggttctgaggaggggcccgtccgggcaaccggcgggtctactcaccCATATGAATAT | 70°.9 C., 2min |
| | | CCTCCTTAG SEQ ID NO: 21 | |
| | mcbG-VR | CCTAACAACGCCACGACTTT SEQ ID NO: 22 | 49° C., 2min |
| Δ6 | 6-KF | acacatttcgtacagcctttacactcggtgaattagggccctagatgcaGTGTAGGCTGGAG | 67° C., 3min |
| | | CTGCTTC SEQ ID NO: 23 | |
| | 6-KR | ttaaacctcatgttttgtgatatctataatctgtgctttaggtatattatCATATGAATATCCTCC | |
| | | TTAG SEQ ID NO: 24 | |
| | 6-VF | GAAGATATCGCACGCCTCTC SEQ ID NO: 25 | 54.5° C., 3min |
| | 6-VR | CGCCTGTTTGGCTATATGTG SEQ ID NO: 26 | |
| Δ7 | 7-KF | aatatacctaaagcacagattatagatatcacaaaacatgaggtttaaaaGTGTAGGCTGGAG | 70° C., 90s |
| | | CTGCTTC SEQ ID NO: 27 | |
| | 7-KR | tggagtttgtgcaggacgggagaaggaaatttctggttatcccgcaggggCATATGAATATC | |
| | | CTCCTTAG SEQ ID NO: 28 | |
| | 7-VF | TTCGATGAACCGACAAAAGG SEQ ID NO: 29 | 54° C., 2.5min |
| | 7-VR | GGGTGAAAGAGGCGATGAT SEQ ID NO: 30 | |
| Δ8 | 8-KF | tttaccgcagctgcctcgcacgcttcggggatgacggtgaaaacctctgaGTGTAGGCTGGA | 68° C., 90s |
| | | GCTGCTTC SEQ ID NO: 31 | |
| | 8-KR | agcagacaccgctcgccgcagccgaacgaccgagtgtagctagtcagtgaCATATGAATAT | |
| | | CCTCCTTAG SEQ ID NO: 32 | |
| | 8-VF | CACGGAGGCATCAGTGACTA SEQ ID NO: 33 | 54.9° C., 2.5min |
| | 8-VR | CAGCCTTTTCCTGGTTCTTG SEQ ID NO: 34 | |
| ΔABC | ABC-KF | aattctagataacataaagcccgtaatatacgggctttaaggattataaaGTGTAGGCTGGAG | 64.5° C., 90s |
| | | CTGCTTC SEQ ID NO: 35 | |
| | ABC-KR | atatttcttaaatttactatgatttcctffittataagattattcatttCATATGAATATCCTCCTT | |
| | | AG SEQ ID NO: 36 | |
| | ABC-VF | GCGAAAAGATGTTTGGAATGA SEQ ID NO: 37 | 52.7° C., 90s |
| | ABC-VR | TCGGGAAAGTTGTCATTTGC SEQ ID NO: 38 | |
| Δhp1 | hp1-KF | ataaatgataactattctcatctacattcaaatatataattgggggtgttGTGTAGGCTGGAGC | 65.7° C., 90s |
| | | TGCTTC SEQ ID NO: 39 | |
| | hp1-KR | aataaaattcaatttataatccttaaagcccgtatattacgggctttatgCATATGAATATCCTC | |
| | | CTTAG SEQ ID NO: 40 | |
| | hp1-VF | ACTGGCTGCAAAAACCTTGT SEQ ID NO: 41 | 53.2° C., 75s |
| | hp1-VR | TTTCTCCTATTGAATCTTTATTGTCA SEQ ID NO: 42 | |
| Δcupin | cupin KF | aatatacctaaagcacagattatagatatcacaaaacatgaggtttaaaaGTGTAGGCTGGAG | 66.5° C., 3min |
| | | CTGCTTC SEQ ID NO: 43 | |
| | cupin KR | agtttatatcgtatgaaaaaatctaaggggaagcccccttagattaatggCATATGAATATCC | |
| | | TCCTTAG SEQ ID NO: 44 | |
| | cupin VF | AAAGAGGAAAACAAGGAAAAGCA SEQ ID NO: 45 | 54° C., 2min |
| | cupin VR | GCATTGCTTGTGTTTCAGGG SEQ ID NO: 46 | |
| ΔDUF4440 | DUF4440 | aaaaaataaaacttgaacatatataaccattaatctaagggggcttccccGTGTAGGCTGGAG | 68° C., 3min |
| | KF | CTGCTTC SEQ ID NO: 47 | |
| | DUF4440 | aggaatgttggggatagattagaggaggaattagatatgaggaaggtagtCATATGAATATC | |
| | KR | CTCCTTAG SEQ ID NO. 48 | |
| ΔDUF2164 | DUF2164 | tgcattataccattcttttcttattagatttaagtctgatttaaaattagGTGTAGGCTGGAGCTG | 68° C., 3min |
| | KF | CTTC SEQ ID NO: 49 | |
| | DUF2164 | tgataggaaaaatgttatattattaatttatttgtgaggcttcataaagaCATATGAATATCCTC | |
| | KR | CTTAG SEQ ID NO: 50 | |
| ΔDUF4440 & | DUF4440/ | GGCACAATGTTACGACTCAGA SEQ ID NO: 51 | 55° C., 90s |
| ΔDUF2I64 | 2164 VF | | |
| | DUF4440/ | GTTTCAGCGGTGCGTACAAT SEQ ID NO: 52 | |
| | 2164 VR | | |
| Δh2 | hp2 KF | aaattacaactcaaccatactgcaacctggaatttcccaagcaagcatatGTGTAGGCTGGAG | 68° C., 3min |
| | | CTGCTTC SEQ ID NO: 53 | |
| | hp2 KR | tgtctctggctggcaattcctgcgtgattcacatggctgcatagctatgcCATATGAATATCCT | |
| | | CCTTAG SEQ ID NO: 54 | |
| | hp2 VF | TCCTCTGATTCAAACTGTCCAAG SEQ ID NO: 55 | 55° C., 90s |
| | hp2 VR | TGTTGCTGTGTTTTGCCTCT SEQ ID NO: 56 | |
| Δhp3 | hp3 KF | aggcaaaacacagcaacaaaagacacaccagaatcgcgcccgtatgcgttGTGTAGGCTGG | 68° C., 3min |
| | | AGCTGCTTC SEQ ID NO: 57 | |
| | hp3 KR | acagcgagaacaggagataagggatgaacggctgatacaggaacgcgaacCATATGAATA | |
| | | TCCTCCTTAG SEQ ID NO: 58 | |
| | hp3 VF | GAATTGCCAGCCAGAGACAG SEQ ID NO: 59 | 55 C., 90s |
| | hp3 VR | GGTCATGCAGTTGAGTCAGC SEQ ID NO: 60 | |
| ΔtonB | tonB-KF | tgcatttaaaatcgagacctggtttttctactgaaatgattatgacttcaGTGTAGGCTGGAGC | 68.6 C., 90s |
| | | TGCTTC SEQ ID NO: 61 | |
| | tonB-KR | ctgttgagtaatagtcaaaagcctccggtcggaggcttttgactttctgcCATATGAATATCCT | |
| | | CCTTAG SEQ ID NO: 62 | |
| | tonB-VF | AACATACAACACGGGCACAA SEQ ID NO: 63 | 54.9° C., 75s |
| | tonB-VR | GACGACATCGGTCAGCATTA SEQ ID NO: 64 | |
| ΔtolC | tolC-KF | aattttacagtttgatcgcgctaaatactgcttcaccacaaggaatgcaaGTGTAGGCTGGAG | 64.5° C., 90s |
| | | CTGCTTC SEQ ID NO: 65 | |
| | tolC-KR | atctttacgttgccttacgttcagacggggccgaagccccgtcgtcgtcaCATATGAATATCC | |
| | | TCCTTAG SEQ ID NO: 66 | |
| | tolC-VF | CCAAATGTAACGGGCAGGTT SEQ ID NO: 67 | 56° C., 2.5min |
| | tolC-VR | GCGTGGCGTATGGATTTTGT SEQ ID NO: 68 | |
| pBAD18::tolC | pBAD18 | GCTAGCGAATTCGAGCTCGGTACCCGGGGGAATCCGCAATAAT | 62° C., 2.5min |
| | tolC L | TTTACAGTTTGATCGCG SEQ ID NO: 69 | |
| | insert | | |
| | pBAD18 | GCTTGCATGCCTGCAGGTCGACTCTAGAGGATAACCCGTATCT | |
| | tolC R | TTACGTTGCCTTACG SEQ ID NO: 70 | |
| | insert | | |
| | pBAD18 | CGTAAGGCAACGTAAAGATACGGGTTATCCTCTAGAGTCGACC | 62° C., 5min |
| | tolC R | TGCAGGCATGCAAGC SEQ ID NO: 71 | |
| | plasmid | | |
| | pBAD18 | CGCGATCAAACTGTAAAATTATTGCGGATTCCCCCGGGTACCG | |
| | tolC L | AGCTCGAATTCGCTAGC SEQ ID NO: 72 | |
| | plasmid | | |
| | pBAD18 F | CTGTTTCTCCATACCCGTT SEQ ID NO: 73 | 45° C., 2.25min |
| | pBAD18 R | CTCATCCGCCAAAACAG SEQ ID NO: 74 | |
| pBR322:: | pBR322 | GTATATATGAGTAAACTTGGTCTGACAGCATTAAAAGAGGCGT | 56° C., 4min |
| p0.1229_3²⁷⁴⁵⁻⁷⁹⁵⁰ | hp1 | CAGAGGCAGAAAACG SEQ ID NO: 75 | |
| | upstream L | | |
| | insert | | |
| | pBR322 | GCGGCATTTTGCCTTCCTGTTTTTGCGAAATCGGCAACGGTGAT | |
| | end 7 R | TCCCTATCAGGG SEQ ID NO: 76 | |
| | insert | | |
| | pBR322 | CCCTGATAGGGAATCACCGTTGCCGATTTCGCAAAAACAGGAA | 62° C., 4min |
| | end 7 R | GGCAAAATGCCGC SEQ ID NO: 77 | |
| | plasmid | | |
| | pBR322 | CGTTTTCTGCCTCTGACGCCTCTTTTAATGCTGTCAGACCAAGT | |
| | hp1 | TTACTCATATATAC SEQ ID NO: 78 and 79 | |
| | upstream L | | |
| | plasmid | | |
| | pBR322-F | TTTGCAAGCAGCAGATTACG SEQ ID NO: 80 | 54.4° C., 2min |
| | pBR322-R | GCCTCGTGATACGCCTATTT SEQ ID NO: 81 | |

ECRC, Penn State E. coli Reference Center; Amp^{R}, ampicillin resistant; Cat^{R}, chloramphenicol resistant; KanR, kanamycin resistant; TetR, tetracycline resistant; stx2a, Shiga toxin 2a; stx1a, Shiga toxin 1a; P_{araC}, arabinose inducible promoter; Tₐ, amplification temperature; KF, knockout forward; KR, knockout reverse; VF, verification forward; VR, verification reverse. Note: For the KF or KR primers, the lower-case letters indicate homologous regions to the target gene and the upper-case letters indicate the primer for the antibiotic resistant cassette. Superscript numbers indicate regions knocked out.

### EXAMPLE 2

>NODE_34_length_12971_cov_948.013
SEQ ID NO: 1
AAAATACCCGCCGTGAGCATGCAGCGCTGATACGTCAGCA
CTATCAGTATCGTGAATTTGCCTGGCCCTGGACATTTCGC
CTTACCCGTCTTTTATATACCCGGAGCTGGATAAGCAACG
AACGTCCTGGCCTGCTTTTCGATCTGGCGACAGGGTGGCT
TATGCAACATCGTATTATTCTCCCCGGAGCCACTACGCTG
ACCCGGTTGATTTCAGAGGTAAGGGAAAAGGCGACGTTGC
GCCTGTGGAACAAACTGGCACTGATACCGTCAGCCGAACA
GCGTTCACAGCTGGAGATGCTGCTGGGGCCAACTGATTGC
AGCCGCCTGTCTTTACTGGAATCACTGAAAAAGGGCCCTG
TGACCATCAGTGGTCCGGCGTTTAATGAAGCAATTGAACG
CTGGAAAACTCTGAACGATTTTGGCCTGCATGCTGAAAAC
CTGAGTACACTCCCGGCTGTGCGCCTGAAAAATCTCGCAC
GTTATGCTGGTATGACTTCGGTGTTCAATATTGCCAGGAT
GTCACCGCAGAAAAGGATGGCGGTTCTGGTTGCCTTTGTC
CTTGCATGGGAAACGCTGGCGCTGGATGATGCATTGGACG
TTCTGGACGCCATGCTGGCCGTTATCATCCGTGACGCCAG
AAAGATTGGGCAGAAAAAACGGCTCCGCTCGCTGAAGGAT
CTGGATAAATCTGCATTGGCGCTCGCCAGCGCATGTTCGT
ACCTGCTGAAAGAAGAAACACCGGACGAATCGATTCGTGC
TGAGGTGTTCAGCTACATCCCAAGGCAAAAGCTGGCTGAA
ATCATCACGCTTGTCCGTGAAATTGCCCGGCCCTCAGACG
ATAATTTTCATGAAGAAATGGTGGAGCAGTACGGGCGCGT
TCGTCGTTTCCTGCCCCATCTGCTGAATACCGTTAAATTT
TCATCCGCACCTGCCGGGGTTACCACTCTGAATGCCTGTG
ACTACCTCAGCCGGGAGTTCAGCTCACGGCGGCAGTTTTT
TGACGACGCACCAACGGAAATTATCAGTCGGTCATGGAAA
CGGCTGGTGATTAACAAGGAAAAACATATCACCCGCAGGG
GATACACGCTCTGCTTTCTCAGTAAACTGCAGGATAGTCT
GAGGCGGAGGGATGTCTACGTTACCGGCAGTAACCGGTGG
GGAGATCCTCGTGCAAGATTACTACAGGGTGCTGACTGGC
AGGCAAACCGGATTAAGGTTTATCGTTCTTTGGGGCACCC
GACAGACCCGCAGGAAGCAATAAAATCTCTGGGTCATCAG
CTTGATAGTCGTTACAGACAGGTTGCTGCACGTCTTTGCG
AAAATGAGGCTGTCGAACTCGATGTTTCTGGCCCGAAGCC
CCGGTTGACAATTTCTCCCCTCGCCAGTCTTGATGAGCCG
GACAGTCTGAAACGACTGAGCAAAATGATCAGTGATCTAC
TCCCTCCGGTGGATTTAACGGAGTTGCTGCTCGAAATTAA
CGCCCATACCGGATTTGCTGATGAGTTTTTCCATGCTAGT
GAAGCCAGTGCCAGAGTTGATGATCTGCCCGTCAGCATCA
GCGCCGTGCTGATGGCTGAAGCCTGCAATATCGGTCTGGA
ACCACTGATCAGATCAAATGTTCCTGCACTGACCCGACAC
CGGCTGAACTGGACAAAAGCGAACTATCTGCGGGCTGAAA
CTATCACCAGCGCTAATGCCAGACTGGTTGATTTTCAGGC
AACGCTGCCACTGGCACAGATATGGGGTGGAGGAGAAGTG
GCATCTGCAGATGGAATGCGCTTTGTTACGCCAGTCAGAA
CAATCAATGCCGGACCGAACCGCAAATACTTTGGTAATAA
CAGAGGGATCACCTGGTACAACTTTGTGTCCGATCAGTAT
TCCGGCTTTCATGGCATCGTTATACCGGGGACGCTGAGGG
ACTCTATCTTTGTGCTGGAAGGTCTTCTGGAACAGGAGAC
CGGGCTGAATCCAACCGAAATTATGACCGATACAGCAGGT
GCCAGCGAACTTGTCTTTGGCCTTTTCTGGCTGCTGGGAT
ACCAGTTTTCTCCACGCCTGGCTGATGCCGGTGCTTCGGT
TTTCTGGCGAATGGACCATGATGCCGACTATGGCGTGCTG
AATGATATTGCCAGAGGGCAATCAGATCCCCGAAAAATAG
TCCTTCAGTGGGACGAAATGATCCGGACCGCTGGCTCCCT
GAAGCTGGGCAAAGTACAGGTTTCAGTGCTGGTCCGTTCA
TTGCTGAAAAGTGAACGTCCTTCCGGACTGACTCAGGCAA
TCATTGAAGTGGGGCGCATCAACAAAACGCTGTATCTGCT
TAATTATATTGATGATGAAGATTACCGCCGGCGCATTCTG
ACCCAGCTTAATCGGGGAGAAAGTCGCCATGCCGTTGCCA
GAGCCATCTGTCACGGTCAAAAAGGTGAGATAAGAAAACG
ATATACCGACGGTCAGGAAGATCAACTGGGCACACTGGGG
CTGGTCACTAACGCCGTCGTGTTATGGAACACTATTTATA
TGCAGGCAGCCCTGGATCATCTCCGGGCGCAGGGTGAAAC
ACTGAATGATGAAGATATCGCACGCCTCTCCCCGCTTTGC
CACGGACATATCAATATGCTCGGCCATTATTCCTTCACGC
TGGCAGAACTGGTGACCAAAGGACATCTGAGACCATTAAA
AGAGGCGTCAGAGGCAGAAAACGTTGCTTAACGTGAGTTT
TCGTTCCACTGAGCGTCAGACCCCGGAACCTACAACACAT
GTGTAAAACGTCAATGGAGGGGGCTATTATCGGACTGCAA
CACATTTCGTACAGCCTTTACACTCGGTGAATTAGCGGCC
CTAGATGCATCCACTCATTCAACCAACTCAATTCTCTTCT
CTTAGAGTGGAAAAATTTTGTTTTCCTTGCAGCCCCTACC
CGTAAAAACTGGCTGCAAAAACCTTGTATTACTAATGGAT
CCTATAACCATTAAAAAACTTGATTACTATCTCATTTATA
GAATTAGGCATATTGACACAAGCACAAATTTAACAATACT
ATAATAAATGATAACTATTCTCATCTACATTCAAATATAT
AATTGGGGGTGTTATGCTACAACATAAGATGAACAGCAGT
TCTTATGCAAAAGTTCATAATGTTAGCTCATTAGAAGATA
TCATGAGTTATCACAATGATGATGTTCTTCTAAAATTTCG
TAAAGAATGGAATGTAACACCAGAAGAAGCTGATGATATC
TTTAATGAAACAAAGAAATTCATCTGGCTAGCCTCAACAT
GCCTAACGGAATGCTACAATATAAAAGTTCACGAGCAATT
ACAAATTATAGATGAAATGTGGCATACGTTTATTCAATTT
ACAGATGCTTATACCAGCTTTTGTGAAAAATATCTTGGTG
CTTACCTCCACCATTATCCAAATACAAATGACATGCTAAA
AAATGAGATAAGGCATGTTAATGAGCATGGTATAACATTC
CAAGAATATCGTTTTAACGAATATAAAAATCAAATTGAAA
AAATCGCTTTTTACCTGGGTCATGAAACTGTCGCAAAGTG
GTATGGTGATTATGCTGTAAGATACAGCATAAAGAACATT
AATACTATAAGAATTCCAAAAGAATCCATATCCAGTGATT
CTTACATCGaaaAAGTAAAGAGTATAACTCACCTTCCAGC
CGCAGAATTTGTAAAAATAATAATGCGAAAAGATGTTTGG
AATGATAATGGTTCTGTTTGTGGTTGCAGCGGTAAAGGAT
GTGGCGCTGGGTGCTCATGTAATTCTAGATAACATAAAGC
CCGTAATATACGGGCTTTAAGGATTATAAATTGAATTTTA
TTGaaaAGTACATCATCACATTTAATAAATGTAATTTGTT
TTTTATTATATTATTATCAACTATAAGTAGTTTTCTTTTC
GTTCTATTCGGATATTTAATAAAAACAGTTATTGACAATA
AAGATTCAATAGGAGAAATAAACTCATTATTAATATTTAT
AGCATGTTTCTTATCAATTAGATTCCTTATGCCTGCTGGA
TATAGCATATCGGAATACTTGACTCAAAAAACAAATATAG
AATTATCTGTAAAGCTTAGAGAGCAAGTTATTGATAATAT
ACAAAATTCTCACCAAGAGCATTTTTTAAAGAAAAATAAA
GGAGAACTAAATAAGGTTATAGAAAGCATGCTTTCTTCTG
CATCATCATTATTTTATACAATATGTTCTGATGTAGTACC
ATTATTAATACAAATGATTGGAATAATCATAACAATTTGT
ATAAGTGTTAATACATTAATAGCCATTGAGTTTATAATAA
TAATGGCAATCTATATAATATTTGTCATAAAAATGACACA
ACGTAGATTTCCAATGATGAAATCAGTTGCACTTAGCTCC
AAATTCGCATCTGGAAGAATGTTTGATATGATGCACATGT
ATCCTATGGATAAAGCTTTCCATACCACGGATAAAAGTCG
TAAACGAGTAATTCAAGCTGTAAATACACATTCAGAAAAA
CAAAGAAAAGTAAATAATGAATTTTTCCTGTTTGGTATCA
GCTCCGCTTTTCTTTCAGTCATTTTTAGTAGTCTGATTAT
CCTATCAGCATACTGGATGTTTCTTCATGGTAGAGCAAGC
CGTGGTAGTATTATTATGCTTGCCACTTTTTTATTCCAAG
TTTTTCTCCCATTAAATCGCATTGGTTATCTATTTAGACA
AATAAAAATGGCAAGAACAGAAATTGATTTATACTGTGCC
GAAATAAACGACATAAAAAAAACAAATTACACAGACAAGC
AATATCTTCATGTAAAAGACAATATTTGCAATATTGAAAT
ATACAACAAATCATTCAACCATAAATTAATATTAACAAAA
GGCGTTGTTACCTTTATAACCGGTGAGAATGGTTCGGGAA
AAACAACTATTGCAAAAATTCTCTCTGGCAATATAACAAC
AGAAGAAAACACAATAAAAATCAATGGGATACAACAAGAA
AAAGCAAATTCTCCTTTTGTTAATGTCTTATACGTTCCTC
AAGATCTAGATCTCATGCCTGGAACTATAGAGGAAAATAT
ATTACATTATTCCGGCATTAATAATTTAAATACAATTAAA
AAGCTGCTACACAGATTTAAGTTCGACAAACCACTAGATT
ATGAGATCAAAGGTTACGGACATAATTTATCTGGTGGACA
AAAACAAAAATTAGGAGTATCTCTTACATCTGGAAAAAAC
GTAGACTTGATTATTTTCGATGAACCGACAAAAGGGTTTG
ATTCACTTGGTATAAAAATAATCAGTGATTATATAAAAGA
GGAAAACAAGGAAAAGCATATCATTGTTATATCTCATGAT
GAACAACTTATAAATAATATACCTAAAGCACAGATTATAG
ATATCACAAAACATGAGGTTTAAAAATGAATAATCTTATA
AAAAAGGAAATCATAGAAAAATTTAAGAAATATAATTTCC
AGAAATATCCGTTTGTATTCACGGATGTTAATTATAAAAA
CTTAATCAACTGGAATGATCTTAATAAATTGCTTGAAAAA
GATATATTGCACTATCCTAGAGTTAGAATGGCAAATGACA
ACTTTCCCGAAATTAGGGGGTATAAAGGATTTATAAGATA
CACATATAGCCAAACAGGCGATAGAACACCACATATAAAT
CGCCATCAGCTATATAAATGCTTACGTGATGGCGCAACCC
TTATAGTAGATCGATGCCAGTCATTCTTTGAATCTGTAGA
TGAAAGCAGACTATGGTTATCTAAAGAGTTAGAATGTACA
TGTAGTGCTAATCTATATGCTGCATTTACAGCAACACCAA
GCTTTGGGCTTCATTTTGACAATCATGATGTAATAGCTGT
TCAAATTGAGGGAATAAAAAAATGGAAAGTTTACAACCCT
ACCTATTCATACCCTCTCGAAGATGAAAGAAGCTTCGATT
ATCTACCACCTAATACCTCCCCAGATTATGAGTTTGATAT
AACCCCAGGTCAGGCCATATATCTTCCTGCTGGATACTGG
CACAATGTTACGACTCAGAGCAAACACTCTCTTCATATAT
CTTTTACAGTTATAAGACCTCGTCGATTAGAACTATTTAA
AACATTATTTGATGAATTAAAAAACAATCCATATATGCGG
GAACCTATAGAGCATGGTGATTCATTATCTGATAAAGAAA
AAATAAAAACTATAATAACTAATGCTATAAATAGTTTTGA
TATTCAGTTTGCAGAAAACATGCTTAAAGCTCAAAGCAGA
ACTTATCGCTATAAAAAAATAAAACTTGAACATATATAAC
CATTAATCTAAGGGGGCTTCCCCTTAGATTTTTTCATACG
ATATAAACTGCATTTTCCATCCACCATTAACCTTTATCCA
ATTCTCAATAAATGCCATTAATATATTTCCATCATTTTGC
TTTATTTCAGCTTTCCCTGAAACACAAGCAATGCCTTTAA
ACTCACGCATTACGACATCATATTCATATCTTTCTATGCT
AGGTAAAATCCTCGCTGGACGCATATCAATCTTTCGTAAC
TGATGTTTTTTATAGATAACTTTTTGACCATTTGTTGAAA
TAAACCAATCAGCTTCCAAGTAATCTAGCATTTCAGTATT
ACCTGAATAAAATGCATTATACCATTCTTTTCTTATTAGA
TTTAAGTCTGATTTAAAATTAGTCACACTACCTTCCTCAT
ATCTAATTCCTCCTCTAATCTATCCCCAACATTCCTTACT
GATTTTATAGCATCATCAATAGCCACATTATATATGACTG
GGATTACTCTTTTAAGAATCTCATCAAGTAATTCCTCTGA
TTCAAACTGTCCAAGGCATATATCATGTTCATCTCTGATT
TTTTGTGAAATAAATTCAGCTAGTCCTTTATATATGTTTT
TTTCAATATCAGAAATTTTCATTCTTTATGAAGCCTCACA
AATAAATTAATAATATAACATTTTTCCTATCATAGGAAAA
TTACAACTCAACCATACTGCAACCTGGAATTTCCCAAGCA
AGCATATTTAAATAAGTGCAGGATACCACCTCGAATCATA
CTCTAAGGAACAAGATAATTTACCATAATCCCTTATTGTA
CGCACCGCTGAAACGCGTTCGGCGCGATCACGGCAGCAGA
CAGGTAAAAATGGCAACAAACCACCCGAAAAACTGCCGCG
ATCGCACCCGGTAAATTTTAACCACATGCATAGCTATGCA
GCCATGTGAATCACGCAGGAATTGCCAGCCAGAGACAGCT
GAAACGGATTTTTTTCATGCTTTCGGAAGCGAAGAAGACG
GGGACCGGAGCGGGAAAACAGAAGTTCAGGGGAATGAGCG
CGATCTGGCAATAGAGGCAAAACACAGCAACAAAAGACAC
ACCAGAATCGCGCCCGTATGCGTTTTAACGCGTTCCTGTG
TTTTCAGCAAGGTCTGACTGAAGCGCGTTCCCGGTGCCCC
CGAACCAACCATTATCCCAAAAAATGCACCGGAAACGCGC
TGACAACATTTTAGCCTGTCTCTGATTACCATCCCAGCGA
AGGGCCATCCTGTGTCCGTTCCTGTATTTCCAGCTGGCGC
TCACGCTCCAGGGATAACTCATGTTCGCGTTCCTGTATCA
GCCGTTCATCCCTTATCTCCTGTTCTCGCTGTATAACTGG
CTCAAGCGTTCTGTCTGCTCGCTCAAGTGCTGCACCTGCT
GACTCAACTGCATGACCCGCTCGTTCAGCATCGCGTTGTC
CCGTTGCGTAAGCGAAAACATTTTCTGCAATTCCACGAAG
GCGCTCTCCCATTCGTTCAGCCGCTGCATATAGTCCTGTT
GCAGTTGCTCTAAGGCGTTCAGCAAATGTCTTTCCAGCTC
CGTCACTCTGTGTCACTCCGTCAGTTGTACCCAGTCCTTC
CCCTGATAGGGAATCACCGTTGCCGATTTCCCCTGCGGGA
TAACCAGAAATTTCCTTCTCCCGTCCTGCACAAACTCCAC
GCCCCATGTCTTCGCGTTCAGTTTCTGCAATGTTTCTTCC
TGTATCCTGATTTCTTCCAGGTTCGCCTGTATCATCCCGC
CAAGATACCAGAGCGTCCCGCCACTCACGGTAAACAGGAA
AAGGACCATCCCCAGTAACATCATGCCCGTATTCCCTGCC
AGCTTTAACACGTCCTTCCTGTGCTGCATCATCGCCTCTT
TCACCCCTTCCCGGTGTTTTTTCAGTGATTCCTCTGTCGA
AGCTGTGAACAGGGCTATAGCGTCTTTGATTTTCGTCTCG
TTTGATGTCACAGCCTTGCTTACAGATTTTTCGAGCTTGC
TGAACTCGTCGTTCAGCATTGTCTCTGTAGATTCGGCTCT
CTCTTTCAGCTTTTTCTCGAACTCCGCGCCCGTCTGTAAA
AGATTGCTCATAAAATGCTCCTTTCAGCCTGATATTCTTC
CCACCGTTCGGGTCTGCAATGCTGATACTGCTTCGCATCA
CCCTGACCACTTCAAGCCCTGCCTCTGTGAGCGCCTGAAT
CACATCCTGACGGCCTTTTATCTCCCCGACATGATAAAGA
GCGTCTATCCCGCGTGTGACGCTCTCTGCAAGCGCCTGTT
TCGTTTCCGGCAGGTTATCAGGGAGAGTCAGTATCCGGCG
GTTCTCCGGGGCGTTGGGGTCATGCAGCCCGTAATGGTGA
TTAACCAGCGTCTGCCAGGCATCAATTCTCGGCCTGTCTG
CCCGGTCGTAGTACGGCTGGAGGCGTTTTCCGGTCTGTAG
CTCCATGTTCGGGATGACAAAATTCAGCTCAAGCCGTCCC
TTGTCCTGGTGCTCCACCCACAGGATGCTGTACTGATTCT
TTTCGAGACCGGGCATCAGTACACGCTCAAAGCTCGCCAT
CACCTTTTCACGTCCTCCCGGCGGCAGCTCCTTCTCCGCG
AACGACAGAACACCTGACGTGTATTTCTTCGCAAATGGCG
TGGCATCGATGAGTTCCCGAACTTCTTCCGGATTACCCTG
AAGCACCGTTGCCCCTTCCCGGTTTCGCTCCCTTCCCAGC
AGGTAATCAACCGGACCACTGCCACCGCCTTTTCCCCTGG
CATGAAATTTAACAATCATTCCGCGCTCCCTGTTCCCTGA
CGACCTGCCGTAAGCTGCACAACTCTCTCTCGATGGCCAT
CAGTGCGGCCACCACCTGAACCCGGTCACTGGAAGACCAC
TGCCCGCTATTCACCTTCCTCGCTGTCTGATTCAGGTTAT
TCCCGATGGCGGCCAGCTGACGCAGTAACGGCGGTGCCAG
TGTCGGCAGCTTCCCGGAGCGTGCAACCGGCTCACCCAGG
CAGACCCGCCGCATCCATACCGCCAGCTGTTTACCCTCAC
AGCGTTCCAGTAACCGGGCATGTTCATCATCAGTAACCCG
TATGGTGAGCATCCTCTCGCGTTTCATCGGTATCATTACC
CCATAAAACAGAAATCCCCCTTACACGGAGGCATCAGTGA
CTAAACAGGAAAAAACCGCCCTTAACATGGCCCGTTTTAT
CAGAAGCCAGACATTAACGCTGCTGGAGAAACTCAACGAA
CTGGACGCAGATGAACAGGCCGATATTTGTGAATCACTTC
ACGACCACGCCGACGAGCTTTACCGCAGCTGCCTCGCACG
CTTCGGGGATGACGGTGAAAACCTCTGACACATACAGCTC
CCGGAGACGGTCACAGCTTGTCTGTGAGCGGATGCCGGGA
GCAGACAAGCCCGTCAGGGCGCGTCAGGGGGTTTCGGGGC
GAAGCCCTGAACCAGTCACGTAGCGATAGCGGAGTGTATA
CTGGCTTAACTATGCGGCATCAGTGCGAATTGTATGGAAA
GCGCACCATGTCCGATGTGAAATGCCGCACAGATGCGTAA
GGAGAAAATGCACGTTCCGGCGCTCTTCCGCTTCCTCGCT
CACTGACTAGCTACACTCGGTCGTTCGGCTGCGGCGAGCG
GTGTCTGCTCACTCAAAAGCGGAGGTGCGGTTATCCACAG
AATCAGGGGATAACGCCAAAAGAAACATGTGAGCAAAAAA
CAAGAACCAGGAAAAGGCTGCGCCGTTGGCGTTTTTCCAT
AGGCTCCGCCCCCCTGACGAGCATCATAAAAATCGACGCT
CAAGTCAGAGGTGGCGAAACCCGACAGGACTATAAAGATA
CCAGGCGTTTCCCCCTGGAAGCTCCCTCGTGCGCTCTCCT
GTTCCGACCCTGCCGCTTACCGGATACCTGTCCGCCTTTC
TCCCTTCGGGAAGCGTGGCGCTTTCTCATAGCTCACGCTG
TAGGTATCTCGGCTCGGTGTAGGTCGTTCGCTCCAAGCTG
GGCTGTGTGCACGAACCCCCCGTTCAGCCCGACCGCTGCG
CCTTATCCGGTAACTATCGTCTTGAGTCCAACCCGGTAAG
ACACGACAAAGCGCCACTGGCAGCAGCCACTGGTAACTGG
ACAAGGTGGATTGAGATATTAAGAGTTCTTGAAGTGGTGG
CCTAACTGCGGCTACACTAGAAGGACAGTATTTGGTATCT
GTGCTCCACCAAGCCAGTTACCCGGTTAAGCAGTCCCCAA
CTGACTTAACCTTCGACTAAACCGCCTCCCCAGGCGGTTT
TTTCGTTTACTGGCAGCAGATTACGCGCAGAAAAAAAGGA
TCTCAAGAAGATCCTTTGATCTTTTTGAGATCCGCCCACT
CCTGGAACGGGGTCTGACGCTCAGTGGAACGAAAACTCAC
GTTAAGGGATTTTGGTCATGAGATTATCAAAAAGGATCTT
CACCTAGATCCTTTTAAATTAAAAATGAAGTTTTAAATCA
ATCTAAAGTATATATGAGTAAACTTGGTCTGACAGTTACC
AATGCTTAATCAGTGAGGCACCTATCTCAGCGATCTGTCT
ATTTCGTTCATCCATAGTTGCCTGACTCCCCGTCGTGTAG
ATAACTACGATACGGGAGGGCTTACCATCTGGCCCCAGTG
CTGCAATGATACCGCGAGACCCACGCTCACCGGCTCCAGA
TTTATCAGCAATAAACCAGCCAGCCGGAAGGGCCGAGCGC
AGAAGTGGTCCTGCAACTTTATCCGCCTCCATCCAGTCTA
TTAATTGTTGCCGGGAAGCTAGAGTAAGTAGTTCGCCAGT
TAATAGTTTGCGCAACGTTGTTGCCATTGCTGCAGGCATC
GTGGTGTCACGCTCGTCGTTTGGTATGGCTTCATTCAGCT
CCGGTTCCCAACGATCAAGGCGAGTTACATGATCCCCCAT
GTTGTGCAAAAAAGCGGTTAGCTCCTTCGGTCCTCCGATC
GTTGTCAGAAGTAAGTTGGCAGCAGTGTTATCACTCATGG
TTATGGCAGCACTGCATAATTCTCTTACTGTCATGCCATC
CGTAAGATGCTTTTCTGTGACTGGTGAGTACTCAACCAAG
TCATTCTGAGAATAGTGTATGCGGCGACCGAGTTGCTCTT
GCCCGGCGTCAACACGGGATAATACCGCACCACATAGCAG
AACTTTAAAAGTGCTCATCATTGGAAAACGTTCTTCGGGG
CGAAAACTCTCAAGGATCTTACCGCTGTTGAGATCCAGTT
CGATGTAACCCACTCGTGCACCCAACTGATCTTCAGCATC
TTTTACTTTCACCAGCGTTTCTGGGTGAGCAAAAACAGGA
AGGCAAAATGCCGCAAAAAAGGGAATAAGGGCGACACGAA
AATGTTGAATACTCATACTCTTCCTTTTTCAATATTATTG
AAGCATTTACCAGGGTTATTGTCTCATGAGCGGATACATA
TTTGAATGTATTTAGAAAAATAAACAAATAGGGGTTCCGC
GCACATTTCCCCGAAAAGTGCCACCTGACGTCTAAGAAAC
CATTATTATCATGACATTAACCTATAAAAATAGGCGTATC
ACGAGGCCCTTTCGTCTTCAAGAATTTTATAAACCGTGGA
GCGGGCAATACTGAGCTGATGAGCAATTTCCGTTGCACCA
GTGCCCTTCTGATGAAGCGTCAGCACGACGTTCCTGTCCA
CGGTACGCCTGCGGCCAAATTTGATTCCTTTCAGCTTTGC
TTCCTGTCGGCCCTCATTCGTGCGTTCTAGGATCCTCCGG
CGTTCAGCCTGTGCCACAGCCGACAGGATGGTGACCACCA
TTTGCCCCATATCACCGTCGGTACTGATCCCGTCATCAAT
GAACCGGACTGCCACGCCCTGAGCGTCAAATTCCTTTATC
AGTTGGATCATATCGGCAGTGTCGCGGCCAAGACGGTCGA
GCTTCTTAACCAGAATGACATCACCTTCCTCCACCTTCAT
CCTCAGCAAATCCAGCCCTTCCCGGTCTGTTGAACTGCCG
GATGCCTTATCGGTAAATATACGGTTTGCTTTCACACCTG
CGTCTTTGAGTGCTCTGACCTGAAGATCAAGAGACTGCTG
ACTGGTTGAGACCCGAGCGTAACCAAAAAGTCGCATAAAA
ATGTACCTTAAATCGAATATCGGACAACTCATGTCTATTA
TTACAAATTTACGATTTAATAGACATATTAATGTAACAGT
TTTACGATGTCCGATAATTTATAACATTTCGTACGGTTGG
AAAAATGTTACTAAATGCCCGTCAGGCAGGGAGGCCGATA
TGCCCGTTGACTTTCTGACCACTGAGCAGACTGAAAGCTA
TGGCAGATTCACCGGTGAACCGGATGAGCTTCAGCTGGCA
CGATATTTTCACCTTGATGAAGCAGACAAGGAATTTATCG
GAAAAAGCAGAGGTGATCACAACCGTCTGGGCATTGCCCT
GCAAATTGGATGTGTCCGTTTTCTGGGCACCTTCCTCACC
GATATGAATCATATTCCTTCCGGCGTCCGGCATTTTACCG
CCAGACAGCTCGGGATTCGTGATATCACCGTTCTTGCAGA
ATACGGTCAGAGGG

### EXAMPLE 3

GenBank CP028323J

LOCUS CP028323 12894 bp DNcircular BCT 11-APR-2019

| | |
|---|---|
| DEFINITION | Escherichia coli O18:H1 strain CFSAN067215 plasmid p0.1229_3, |
| | complete sequence. |
| ACCESSION | CP028323 |
| VERSION | CP028323.1 |
| DBLINK | BioProject: PRJNA230969 |
| | BioSample: SAMN08737532 |
| KEYWORDS | . |
| SOURCE | Escherichia coli O18:H1 |
| ORGANISM | Escherichia coli O18:H1 |
| | Bacteria; Proteobacteria; Gammaproteobacteria; |
| | Enterobacterales; Enterobacteriaceae; Escherichia. |
| REFERENCE | 1 (bases 1 to 12894) |
| | ##Genome-Assembly-Data-START## |
| | Assembly Method:: HGAP v. 3.0 |
| | Genome Representation:: Full |
| | Expected Final Version:: Yes |
| | Genome Coverage:: 405.0x |
| | Sequencing Technology:: PacBio |
| | ##Genome-Assembly-Data-END## |
| | ##Genome-Annotation-Data-START## |
| | Annotation Provider:: NCBI |
| | Annotation Date:: 03/27/2018 11:22:31 |
| | Annotation Pipeline:: NCBI Prokaryotic Genome Annotation Pipeline |
| | Annotation Method:: Best-placedreference protein set; GeneMarkS |
| | Annotation Software revision:: 4.4 |
| | Features Annotated:: Gene; CDS; rRNA; tRNA; ncRNA; repeat_region |
| | Genes (total):: 5,487 |
| | CDS (total):: 5,362 |
| | Genes (coding):: 5,099 |
| | CDS (coding):: 5,099 |
| | Genes (RNA):: 125 |
| | rRNAs:: 8, 7, 7 (5S, 16S, 23S) |
| | complete rRNAs:: 8, 7, 7 (5S, 16S, 23S) |
| | tRNAs:: 97 |
| | ncRNAs:: 6 |
| | Pseudo Genes (total):: 263 |
| | Pseudo Genes (ambiguous residues):: 0 of 263 |
| | Pseudo Genes (frameshifted):: 134 of 263 |
| | Pseudo Genes (incomplete):: 97 of 263 |
| | Pseudo Genes (internal stop):: 67 of 263 |
| | Pseudo Genes (multiple problems):: 32 of 263 |
| | ##Genome-Annotation-Data-END## |
| FEATURES | Location/Qualifiers |
| source | 1..12894 |
| | /organism=″Escherichia coli O18:H1″ |
| | /mol_type=″genomic DNA″ |
| | /strain=″CFSAN067215″ |
| | /host=″Homo sapiens″ |
| | /db_xref=″taxon: 2126982″ |
| | /plasmid=″p0.1229_3″ |
| | /country=″USA:CA″ |
| | /collection_date=″2000″ |
| | /collected_by=″Dudley Lab/Penn State″ |
| gene | join(12600..12894,1..2711) |
| | /locus_tag=″C7X15_27360″ |
| CDS | join(12600..12894,1..2711) |
| | /locus_tag=″C7X15_27360″ |
| | /inference=″COORDINATES: similar to AA |
| | sequence:RefSeq: WP_001496645.1″ |
| | /note=″Derived by automated computational analysis |
| | using |
| | gene prediction method: Protein Homology.″ |
| | /codon_start=1 |
| | /transl_table=11 |
| | /product=″Tn3-like element Tn3 family transposase″ |
| | /protein_id=″QBZ10882,1″ |
| | SEQ ID NO: 3 |

/translation=″MPVDFLTTEQTESYGRFTGEPDELQLARYFHLDEADKEFIGKSR
GDHNRLGIALQIGCVRFLGTFLTDMNHIPSGVRHFTARQLGIRDITVLAEYGQRENTR
REHAALIRQHYQYREFAWPWTFRLTRLLYTRSWISNERPGLLFDLATGWLMQHRIILP
GATTLTRLISEVREKATLRLWNKLALIPSAEQRSQLEMLLGPTDCSRLSLLESLKKGP
VTISGPAFNEAIERWKTLNDFGLHAENLSTLPAVRLKNLARYAGMTSVFNIARMSPQK
RMAVLVAFVLAWETLALDDALDVLDAMLAVIIRDARKIGQKKRLRSLKDLDKSALALA
SACSYLLKEETPDESIRAEVFSYIPRQKLAEIITLVREIARPSDDNFHEEMVEQYGRV
RRFLPHLLNTVKFSSAPAGVTTLNACDYLSREFSSRRQFFDDAPTEIISRSWKRLVIN
KEKHITRRGYTLCFLSKLQDSLRRRDVYVTGSNRWGDPRARLLQGADWQANRIKVYRS
LGHPTDPQEAIKSLGHQLDSRYRQVAARLCENEAVELDVSGPKPRLTISPLASLDEPD
SLKRLSKMISDLLPPVDLTELLLEINAHTGFADEFFHASEASARVDDLPVSISAVLMA
EACNIGLEPLIRSNVPALTRHRLNWTKANYLRAETITSANARLVDFQATLPLAQIWGG
GEVASADGMRFVTPVRTINAGPNRKYFGNNRGITWYNFVSDQYSGFHGIVIPGTLRDS
IFVLEGLLEQETGLNPTEIMTDTAGASELVFGLFWLLGYQFSPRLADAGASVFWRMDH
DADYGVLNDIARGQSDPRKIVLQWDEMIRTAGSLKLGKVQVSVLVRSLLKSERPSGLT
QAIIEVGRINKTLYLLNYIDDEDYRRRILTQLNRGESRHAVARAICHGQKGEIRKRYT
DGQEDQLGTLGLVTNAVVLWNTIYMQAALDHLRAQGETLNDEDIARLSPLCHGHINML
GHYSFTLAELVTKGHLRPLKEASEAENVA″

| | |
|---|---|
| gene | 3094..3792 |
| | /locus_tag=″C7X15_27365″ |
| CD3 | 3094..3792 |
| | /locus_tag=″C7X15_27365″ |
| | /inference=″COORDINATES: similar to AA |
| | sequence:RefSeq: WP_000939065.1″ |
| | /note=″Derived by automated computational analysis |
| | using |
| | gene prediction method: Protein Homology.″ |
| | /codon_start=1 |
| | /transl_table=11 |
| | /product=″hypothetical protein″ |
| | /protein_id=″QBZ10883.1″ SEQ ID NO: 4 |

/translation=″MLQHKMNSSSYAKVHNVSSLEDIMSYHNDDVLLKFRKEWNVTPE
EADDIFNETKKFIWLASTCLTECYNIKVHEQLQIIDEMWHTFIQFTDAYTSFCEKYLG
AYLHHYPNTNDMLKNEIRHVNEHGITFQEYRFNEYKNQIEKIAFYLGHETVAKWYGDY
AVRYSIKNINTIRIPKESISSDSYIEKVKSITHLPAAEFVKIIMRKDVWNDNGSVCGC
SGKGCGAGCSCNSR″

| | |
|---|---|
| gene | 3831..5423 |
| | /locus_tag=″C7X15_27370″ |
| CDS | 3831..5423 |
| | /locus_tag=″C7X15_27370″ |
| | /inference=″COORDINATES: similar to AA |
| | sequence:RefSeq: WP_019842142.1″ |
| | /note=″Derived by automated computational analysis |
| | using |
| | gene prediction method: Protein Homology.″ |
| | /codon_start=1 |
| | /transl_table=11 |
| | /product=″ABC transporter ATP-binding protein″ |
| | /protein_id=″QBZ10884.1″ |
| | SEQ ID NO: 5 |

/translation=″MNFIEKYIITFNKCNLFFIILLSTISSFLFVLFGYLIKTVIDNK
DSIGEINSLLIFIACFLSIRFLMPAGYSISEYLTQKTNIELSVKLREQVIDNIQNSHQ
EHFLKKNKGELNKVIESMLSSASSLFYTICSDVVPLLIQMIGIIITICISVNTLIAIE
FIIIMAIYIIFVIKMTQRRFPMMKSVALSSKFASGRMFDMMHMYPMDKAFHTTDKSRK
RVIQAVNTHSEKQRKVNNEFFLFGISSAFLSVIFSSLIILSAYWMFLHGRASRGSIIM
LATFLFQVFLPLNRIGYLFRQIKMARTEIDLYCAEINDIKKTNYTDKQYLHVKDNICN
IEIYNKSFNHKLILTKGVVTFITGENGSGKTTIAKILSGNITTEENTIKINGIQQEKA
NSPFVNVLYVPQDLDLMPGTIEENILHYSGINNLNTIKKLLHRFKFDKPLDYEIKGYG
HNLSGGQKQKLGVSLTSGKNVDLIIFDEPTKGFDSLGIKIISDYIKEENKEKHIIVIS
HDEQLINNIPKAQIIDITKHEV″

| | |
|---|---|
| gene | 5426..6319 |
| | /locus_tag=″C7X15_27375″ |
| CDS | 5426..6319 |
| | /locus_tag=″C7X15_27375″ |
| | /inference=″COORDINATES: similar to AA |
| | sequence:RefSeq: WP_001547179.1″ |
| | /note=″Derived by automated computational analysis |
| | using |
| | gene prediction method: Protein Homology.″ |
| | /codon_start=1 |
| | /transl_table=11 |
| | /product=″cupin″ |
| | /protein_id=″QBZ10885.1″ SEQ ID NO: 6 |

/translation=″MNNLIKKEIIEKFKKYNFQKYPFVFTDVNYKNLINWNDLNKLLE
KDILHYPRVRMANDNFPEIRGYKGFIRYTYSQTGDRTPHINRHQLYKCLRDGATLIVD
RCQSFFESVDESRLWLSKELECTCSANLYAAFTATPSFGLHFDNHDVIAVQIEGIKKW
KVYNPTYSYPLEDERSFDYLPPNTSPDYEFDITPGQAIYLPAGYWHNVTTQSKHSLHI
SFTVIRPRRLELFKTLFDELKNNPYMREPIEHGDSLSDKEKIKTIITNAINSFDIQFA
ENMLKAQSRTYRYKKIKLEHI″

| | |
|---|---|
| gene | complement(6344..6706) |
| | /locus_tag=″C7X15_27380″ |
| CDS | complement(6344..6706) |
| | /locus_tag=″C7X15_27380″ |
| | /inference=″COORDINATES: similar to AA |
| | sequence:RefSeq: WP_001547178.1″ |
| | /note=″Derived by automated computational analysis |
| | using |
| | gene prediction method: Protein Homology.″ |
| | /codon_start=1 |
| | /transl_table=11 |
| | /product=″DUF4440 domain-containing protein″ |
| | /protein_id=″QBZ10886.1″ SEQ ID NO: 7 |

/translation=″MTNFKSDLNLIRKEWYNAFYSGNTEMLDYLEADWFISTNGQKVI
YKKHQLRKIDMRPARILPSIERYEYDVVMREFKGIACVSGKAEIKQNDGNILMAFIEN
WIKVNGGWKMQFISYEKI″

| | |
|---|---|
| gene | complement(6703..6942) |
| | /locus_tag=″C7X15_27385″ |
| CDS | complement(6703..6942) |
| | /locus_tag=″C7X15_27385″ |
| | /inference=″COORDINATES: similar to AA |
| | sequence:RefSeq: WP_000703021.1″ |
| | /note=″Derived by automated computational analysis |
| | using |
| | gene prediction method: Protein Homology.″ |
| | /codon_start=1 |
| | /transl_table=11 |
| | /product=″DUF2164 domain-containing protein″ |
| | /protein_id=″QBZ10887.1″ SEQ ID NO: 8 |

/translation=″MKISDIEKNIYKGLAEFISQKIRDEHDICLGQFESEELLDEILK
RVIPVIYNVAIDDAIKSVRNVGDRLEEELDMRKVV″

| | |
|---|---|
| gene | complement(7048..7227) |
| | /locus_tag=″C7X15_27390″ |
| CDS | complement(7048..7227) |
| | /locus_tag=″C7X15_27390″ |
| | /inference=″COORDINATES: similar to AA |
| | sequence:RefSeq: WP_001386734.1″ |
| | /note=″Derived by automated computational analysis |
| | using |
| | gene prediction method: Protein Homology.″ |
| | /codon_start=1 |
| | /transl_table=11 |
| | /product=″plasmid mobilization protein″ |
| | /protein_id=″QBZ10888.1″ SEQ ID NO: 9 |

/translation=″MWLKFTGCDRGSFSGGLLPFLPVCCRDRAERVSAVRTIRDYGKL
SCSLEYDSRWYPALI″

| | |
|---|---|
| gene | complement(7425..7622) |
| | /locus_tag=″C7X15_27395″ |
| CDS | complement(7425..7622) |
| | /locus_tag=″C7X15_27395″ |
| | /inference=″COORDINATES: similar to AA |
| | sequence:RefSeq: WP_001565221.1″ |
| | /note=″Derived by automated computational analysis |
| | using |
| | gene prediction method: Protein Homology.″ |
| | /codon_start=1 |
| | /transl_table=11 |
| | /product=″hypothetical protein″ |
| | /protein id=″QBZ10889.1″ |
| | SEQ ID NO: 10 |

/translation=″MSYPWSVSASWKYRNGHRMALRWDGNQRQAKMLSARFRCIFWDN
GWFGGTGNALQSDLAENTGTR″

| | |
|---|---|
| gene | complement(7546..9099) |
| | /locus_tag=″C7X15_27400″ |
| CDS | complement(7546..9099) |
| | /locus_tag=″C7X15_27400″ |
| | /inference=″COORDINATES: similar to AA |
| | sequence:RefSeq: WP_001557251.1″ |
| | /note=″Derived by automated computational analysis |
| | using |
| | gene prediction method: Protein Homology.″ |
| | /codon_start=1 |
| | /transl_table=11 |
| | /product=″nuclease″ |
| | /protein_id=″QBZ10890.1″ SEQ ID NO: 11 |

/translation=″MIVKFHARGKGGGSGPVDYLLGRERNREGATVLQGNPEEVRELI
DATPFAKKYTSGVLSFAEKELPPGGREKVMASFERVLMPGLEKNQYSILWVEHQDKGR
LELNFVIPNMELQTGKRLQPYYDRADRPRIDAWQTLVNHHYGLHDPNAPENRRILTLP
DNLPETKQALAESVTRGIDALYHVGEIKGRQDVIQALTEAGLEVVRVMRSSISIADPN
GGKNIRLKGAFYEQSFTDGRGVREKAERESRIYRDNAERRVQQARKICKQGCDIKRDE
NQRRYSPVHSFDRGITEKTPGRGERGDDAAQEGRVKAGREYGHDVTGDGPFPVYREWR
DALVSWRDDTGEPGRNQDTGRNIAETEREDMGRGVCAGREKEISGYPAGEIGNGDSLS
GEGLGTTDGVTQSDGAGKTFAERLRATATGLYAAAERMGERLRGIAENVFAYATGQRD
AERAGHAVESAGAALERADRTLEPVIQREQEIRDERLIQEREHELSLERERQLEIQER
TQDGPSLGW″

| | |
|---|---|
| gene | complement(9089..9412) |
| | /locus_tag=″C7X15_27405″ |
| CD3 | complement(9089..9412) |
| | /locus_tag=″C7X15_27405″ |
| | /inference=″COORDINATES: similar to AA |
| | sequence:RefSeq: WP_000955993.1″ |
| | /note=″Derived by automated computational analysis |
| | using |
| | gene prediction method: Protein Homology.″ |
| | /codon_start=1 |
| | /transl_table=11 |
| | /product=″plasmid mobilization relaxosome protein |
| | MobC″ |
| | /protein_id=″QBZ10891.1″ SEQ ID NO: 12 |

/translation=″MLTIRVTDDEHARLLERCEGKQLAVWMRRVCLGEPVARSGKLPT
LAPPLLRQLAAIGNNLNQTARKVNSGQWSSSDRVQVVAALMAIERELCSLRQVVREQG
ARNDC″

| | |
|---|---|
| gene | 9477..9668 |
| | /locus_tag=″C7X15_27410″ |
| CDS | 9477..9668 |
| | /locus_tag=″C7X15_27410″ |
| | /inference=″COORDINATES: similar to AA |
| | sequence:RefSeq: WP_000165985.1″ |
| | /note=″Derived by automated computational analysis |
| | using |
| | gene prediction method: Protein Homology.″ |
| | /codon_start=1 |
| | /transl_table=11 |
| | /product=″Rop family plasmid primer RNA-binding |
| | protein″ |
| | /protein_id=″QBZ10892.1″ SEQ ID NO: 13 |

/translation=″MTKQEKTALNMARFIRSQTLTLLEKLNELDADEQADICESLHDH
ADELYRSCLARFGDDGENL″

| | |
|---|---|
| gene | complement(9920..10159) |
| | /locus_tag=″C7X15_27415″ |
| CDS | complement(9920..10159) |
| | /locus_tag=″C7X15_27415″ |
| | /inference=″COORDINATES: similar to AA |
| | sequence:RefSeq: WP_001399822.1″ |
| | /note=″Derived by automated computational analysis |
| | using |
| | gene prediction method: Protein Homology.″ |
| | /codon_start=1 |
| | /transl_table=11 |
| | /product=″hypothetical protein″ |
| | /protein_id=″QBZ10892.1″ SEQ ID NO: 14 |

/translation=″MFIVLSGFATSDLSVDFYDARQGGGAYGKTPTAQPFPGSCFLLT
CFFWRYPLILWITAPPLLSEQTPLAAAERPSVASQ″

| | |
|---|---|
| gene | complement(10150..10417) |
| | /locus_tag=″C7X15_27420″ |
| | /pseudo |
| CDS | complement(10150..10417) |
| | /locus_tag=″C7X15_27420″ |
| | /inference=″COORDINATES: similar to AA |
| | sequence:RefSeq: WP_012000844.1″ |
| | /note=″frameshifted; Derived by automated |
| | computational |
| | analysis using gene prediction method: Protein |
| | Homology.″ |
| | /pseudo |
| | /codon_start=1 |
| | /transl_table=11 |
| | /product=″hypothetical protein″ |
| | 10327..10569 |
| | /locus_tag=″C7X15_27425″ |
| | /pseudo |
| CD3 | 10327..10569 |
| | /locus_tag=″C7X15_27425″ |
| | /inference=″COORDINATES: similar to AA |
| | sequence:RefSeq: WP_006573365.1″ |
| | /note=″frameshifted; internal stop; Derived by |
| | automated |
| | computational analysis using gene prediction method: |
| | Protein Homology.″ |
| | /pseudo |
| | /codon_start=1 |
| | /transl_table=11 |
| | /product=″hypothetical protein″ |
| gene | complement(10836..11696) |
| | /gene=″blaTEM″ |
| | /locus_tag=″C7X15_27430″ |
| CDS | complement(10836..11696) |
| | /gene=″blaTEM″ |
| | /locus_tag=″C7X15_27430″ |
| | /inference=″COORDINATES: similar to AA |
| | sequence:RefSeq: WP_032489893.1″ |
| | /note=″Derived by automated computational analysis |
| | using |
| | gene prediction method: Protein Homology.″ |
| | /codon_start=1 |
| | /transl_table=1 |
| | /product=″class A broad-spectrum beta-lactamase TEM-1″ |
| | /protein_id='QBZ10894.1″ SEQ ID NO: 15 |

/translation=″MSIQHFRVALIPFFAAFCLPVFAHPETLVKVKDAEDQLGARVGY
IELDLNSGKILESFRPEERFPMMSTFKVLLCGAVLSRVDAGQEQLGRRIHYSQNDLVE
YSPVTEKHLTDGMTVRELCSAAITMSDNTAANLLLTTIGGPKELTAFLHNMGDHVTRL
DRWEPELNEAIPNDERDTTMPAAMATTLRKLLTGELLTLASRQQLIDWMEADKVAGPL
LRSALPAGWFIADKSGAGERGSRGIIAALGPDGKPSRIVVIYTTGSQATMDERNRQIA
EIGASLIKHW″

| | |
|---|---|
| gene | complement(11879..12436) |
| | /locus_tag=″C7X15_27435″ |
| CD3 | complement(11879..12436) |
| | /locus_tag=″C7X15_27435″ |
| | /inference=″COORDINATES: similar to AA |
| | sequence:RefSeq: WP_005687610.1″ |
| | /note=″Derived by automated computational analysis |
| | using |
| | gene prediction method: Protein Homology.″ |
| | /codon_start=1 |
| | /transl_table=11 |
| | /product=″recombinase″ |
| | /protein_id=″QBZ10895.1″ |
| | SEQ ID NO: 16 |

/translation=″MRLFGYARVSTSQQSLDLQVRALKDAGVKANRIFTDKASGSSTD
REGLDLLRMKVEEGDVILVKKLDRLGRDTADMIQLIKEFDAQGVAVRFIDDGISTDGD
MGQMVVTILSAVAQAERRRILERTNEGRQEAKLKGIKFGRRRTVDRNVVLTLHQKGTG
ATEIAHQLSIARSTVYKILEDERAS″
ORIGIN SEQ ID NO: 2

| | |
|---|---|
| 1 | aaaatacccg ccgtgagcat gcagcgctga tacgtcagca ctatcagtat cgtgaatttg |
| 61 | cctggccctg gacatttcgc cttacccgtc ttttatatac ccggagctgg ataagcaacg |
| 121 | aacgtcctgg cctgcttttc gatctggcga cagggtggct tatgcaacat cgtattattc |
| 181 | tccccggagc cactacgctg acccggttga tttcagaggt aagggaaaag gcgacgttgc |
| 241 | gcctgtggaa caaactggca ctgataccgt cagccgaaca gcgttcacag ctggagatgc |
| 301 | tgctggggcc aactgattgc agccgcctgt ctttactgga atcactgaaa aagggccctg |
| 361 | tgaccatcag tggtccggcg tttaatgaag caattgaacg ctggaaaact ctgaacgatt |
| 421 | ttggcctgca tgctgaaaac ctgagtacac tcccggctgt gcgcctgaaa aatctcgcac |
| 481 | gttatgctgg tatgacttcg gtgttcaata ttgccaggat gtcaccgcag aaaaggatgg |
| 541 | cggttctggt tgcctttgtc cttgcatggg aaacgctggc gctggatgat gcattggacg |
| 601 | ttctggacgc catgctggcc gttatcatcc gtgacgccag aaagattggg cagaaaaaac |
| 661 | ggctccgctc gctgaaggat ctggataaat ctgcattggc gctcgccagc gcatgttcgt |
| 721 | acctgctgaa agaagaaaca ccggacgaat cgattcgtgc tgaggtgttc agctacatcc |
| 781 | caaggcaaaa gctggctgaa atcatcacgc ttgtccgtga aattgcccgg ccctcagacg |
| 841 | ataattttca tgaagaaatg gtggagcagt acgggcgcgt tcgtcgtttc ctgccccatc |
| 901 | tgctgaatac cgttaaattt tcatccgcac ctgccggggt taccactctg aatgcctgtg |
| 961 | actacctcag ccgggagttc agctcacggc ggcagttttt tgacgacgca ccaacggaaa |
| 1021 | ttatcagtcg gtcatggaaa cggctggtga ttaacaagga aaaacatatc acccgcaggg |
| 1081 | gatacacgct ctgctttctc agtaaactgc aggatagtct gaggcggagg gatgtctacg |
| 1141 | ttaccggcag taaccggtgg ggagatcctc gtgcaagatt actacagggt gctgactggc |
| 1201 | aggcaaaccg gattaaggtt tatcgttctt tggggcaccc gacagacccg caggaagcaa |
| 1261 | taaaatctct gggtcatcag cttgatagtc gttacagaca ggttgctgca cgtctttgcg |
| 1321 | aaaatgaggc tgtcgaactc gatgtttctg gcccgaagcc ccggttgaca atttctcccc |
| 1381 | tcgccagtct tgatgagccg gacagtctga aacgactgag caaaatgatc agtgatctac |
| 1441 | tccctccggt ggatttaacg gagttgctgc tcgaaattaa cgcccatacc ggatttgctg |
| 1501 | atgagttttt ccatgctagt gaagccagtg ccagagttga tgatctgccc gtcagcatca |
| 1561 | gcgccgtgct gatggctgaa gcctgcaata tcggtctgga accactgatc agatcaaatg |
| 1621 | ttcctgcact gacccgacac cggctgaact ggacaaaagc gaactatctg cgggctgaaa |
| 1681 | ctatcaccag cgctaatgcc agactggttg attttcaggc aacgctgcca ctggcacaga |
| 1741 | tatggggtgg aggagaagtg gcatctgcag atggaatgcg ctttgttacg ccagtcagaa |
| 1801 | caatcaatgc cggaccgaac cgcaaatact ttggtaataa cagagggatc acctggtaca |
| 1861 | actttgtgtc cgatcagtat tccggctttc atggcatcgt tataccgggg acgctgaggg |
| 1921 | actctatctt tgtgctggaa ggtcttctgg aacaggagac cgggctgaat ccaaccgaaa |
| 1981 | ttatgaccga tacagcaggt gccagcgaac ttgtctttgg ccttttctgg ctgctgggat |
| 2041 | accagttttc tccacgcctg gctgatgccg gtgcttcggt tttctggcga atggaccatg |
| 2101 | atgccgacta tggcgtgctg aatgatattg ccagagggca atcagatccc cgaaaaatag |
| 2161 | tccttcagtg ggacgaaatg atccggaccg ctggctccct gaagctgggc aaagtacagg |
| 2221 | tttcagtgct ggtccgttca ttgctgaaaa gtgaacgtcc ttccggactg actcaggcaa |
| 2281 | tcattgaagt ggggcgcatc aacaaaacgc tgtatctgct taattatatt gatgatgaag |
| 2341 | attaccgccg gcgcattctg acccagctta atcggggaga aagtcgccat gccgttgcca |
| 2401 | gagccatctg tcacggtcaa aaaggtgaga taagaaaacg atataccgac ggtcaggaag |
| 2461 | atcaactggg cacactgggg ctggtcacta acgccgtcgt gttatggaac actatttata |
| 2521 | tgcaggcagc cctggatcat ctccgggcgc agggtgaaac actgaatgat gaagatatcg |
| 2581 | cacgcctctc cccgctttgc cacggacata tcaatatgct cggccattat tccttcacgc |
| 2641 | tggcagaact ggtgaccaaa ggacatctga gaccattaaa agaggcgtca gaggcagaaa |
| 2701 | acgttgctta acgtgagttt tcgttccact gagcgtcaga ccccggaacc tacaacacat |
| 2761 | gtgtaaaacg tcaatggagg gggctattat cggactgcaa cacatttcgt acagccttta |
| 2821 | cactcggtga attagcggcc ctagatgcat ccactcattc aaccaactca attctcttct |
| 2881 | cttagagtgg aaaaattttg ttttccttgc agcccctacc cgtaaaaact ggctgcaaaa |
| 2941 | accttgtatt actaatggat cctataacca ttaaaaaact tgattactat ctcatttata |
| 3001 | gaattaggca tattgacaca agcacaaatt taacaatact ataataaatg ataactattc |
| 3061 | tcatctacat tcaaatatat aattgggggt gttatgctac aacataagat gaacagcagt |
| 3121 | tcttatgcaa aagttcataa tgttagctca ttagaagata tcatgagtta tcacaatgat |
| 3181 | gatgttcttc taaaatttcg taaagaatgg aatgtaacac cagaagaagc tgatgatatc |
| 3241 | tttaatgaaa caaagaaatt catctggcta gcctcaacat gcctaacgga atgctacaat |
| 3301 | ataaaagttc acgagcaatt acaaattata gatgaaatgt ggcatacgtt tattcaattt |
| 3361 | acagatgctt ataccagctt ttgtgaaaaa tatcttggtg cttacctcca ccattatcca |
| 3421 | aatacaaatg acatgctaaa aaatgagata aggcatgtta atgagcatgg tataacattc |
| 3481 | caagaatatc gttttaacga atataaaaat caaattgaaa aaatcgcttt ttacctgggt |
| 3541 | catgaaactg tcgcaaagtg gtatggtgat tatgctgtaa gatacagcat aaagaacatt |
| 3601 | aatactataa gaattccaaa agaatccata tccagtgatt cttacatcga aaaagtaaag |
| 3661 | agtataactc accttccagc cgcagaattt gtaaaaataa taatgcgaaa agatgtttgg |
| 3721 | aatgataatg gttctgtttg tggttgcagc ggtaaaggat gtggcgctgg gtgctcatgt |
| 3781 | aattctagat aacataaagc ccgtaatata cgggctttaa ggattataaa ttgaatttta |
| 3841 | ttgaaaagta catcatcaca tttaataaat gtaatttgtt ttttattata ttattatcaa |
| 3901 | ctataagtag ttttcttttc gttctattcg gatatttaat aaaaacagtt attgacaata |
| 3961 | aagattcaat aggagaaata aactcattat taatatttat agcatgtttc ttatcaatta |
| 4021 | gattccttat gcctgctgga tatagcatat cggaatactt gactcaaaaa acaaatatag |
| 4081 | aattatctgt aaagcttaga gagcaagtta ttgataatat acaaaattct caccaagagc |
| 4141 | attttttaaa gaaaaataaa ggagaactaa ataaggttat agaaagcatg ctttcttctg |
| 4201 | catcatcatt attttataca atatgttctg atgtagtacc attattaata caaatgattg |
| 4261 | gaataatcat aacaatttgt ataagtgtta atacattaat agccattgag tttataataa |
| 4321 | taatggcaat ctatataata tttgtcataa aaatgacaca acgtagattt ccaatgatga |
| 4381 | aatcagttgc acttagctcc aaattcgcat ctggaagaat gtttgatatg atgcacatgt |
| 4441 | atcctatgga taaagctttc cataccacgg ataaaagtcg taaacgagta attcaagctg |
| 4501 | taaatacaca ttcagaaaaa caaagaaaag taaataatga atttttcctg tttggtatca |
| 4561 | gctccgcttt tctttcagtc atttttagta gtctgattat cctatcagca tactggatgt |
| 4621 | ttcttcatgg tagagcaagc cgtggtagta ttattatgct tgccactttt ttattccaag |
| 4681 | tttttctccc attaaatcgc attggttatc tatttagaca aataaaaatg gcaagaacag |
| 4741 | aaattgattt atactgtgcc gaaataaacg acataaaaaa aacaaattac acagacaagc |
| 4801 | aatatcttca tgtaaaagac aatatttgca atattgaaat atacaacaaa tcattcaacc |
| 4861 | ataaattaat attaacaaaa ggcgttgtta cctttataac cggtgagaat ggttcgggaa |
| 4921 | aaacaactat tgcaaaaatt ctctctggca atataacaac agaagaaaac acaataaaaa |
| 4981 | tcaatgggat acaacaagaa aaagcaaatt ctccttttgt taatgtctta tacgttcctc |
| 5041 | aagatctaga tctcatgcct ggaactatag aggaaaatat attacattat tccggcatta |
| 5101 | ataatttaaa tacaattaaa aagctgctac acagatttaa gttcgacaaa ccactagatt |
| 5161 | atgagatcaa aggttacgga cataatttat ctggtggaca aaaacaaaaa ttaggagtat |
| 5221 | ctcttacatc tggaaaaaac gtagacttga ttattttcga tgaaccgaca aaagggtttg |
| 5281 | attcacttgg tataaaaata atcagtgatt atataaaaga ggaaaacaag gaaaagcata |
| 5341 | tcattgttat atctcatgat gaacaactta taaataatat acctaaagca cagattatag |
| 5401 | atatcacaaa acatgaggtt taaaaatgaa taatcttata aaaaaggaaa tcatagaaaa |
| 5461 | atttaagaaa tataatttcc agaaatatcc gtttgtattc acggatgtta attataaaaa |
| 5521 | cttaatcaac tggaatgatc ttaataaatt gcttgaaaaa gatatattgc actatcctag |
| 5581 | agttagaatg gcaaatgaca actttcccga aattaggggg tataaaggat ttataagata |
| 5641 | cacatatagc caaacaggcg atagaacacc acatataaat cgccatcagc tatataaatg |
| 5701 | cttacgtgat ggcgcaaccc ttatagtaga tcgatgccag tcattctttg aatctgtaga |
| 5761 | tgaaagcaga ctatggttat ctaaagagtt agaatgtaca tgtagtgcta atctatatgc |
| 5821 | tgcatttaca gcaacaccaa gctttgggct tcattttgac aatcatgatg taatagctgt |
| 5881 | tcaaattgag ggaataaaaa aatggaaagt ttacaaccct acctattcat accctctcga |
| 5941 | agatgaaaga agcttcgatt atctaccacc taatacctcc ccagattatg agtttgatat |
| 6001 | aaccccaggt caggccatat atcttcctgc tggatactgg cacaatgtta cgactcagag |
| 6061 | caaacactct cttcatatat cttttacagt tataagacct cgtcgattag aactatttaa |
| 6121 | aacattattt gatgaattaa aaaacaatcc atatatgcgg gaacctatag agcatggtga |
| 6181 | ttcattatct gataaagaaa aaataaaaac tataataact aatgctataa atagttttga |
| 6241 | tattcagttt gcagaaaaca tgcttaaagc tcaaagcaga acttatcgct ataaaaaaat |
| 6301 | aaaacttgaa catatataac cattaatcta agggggcttc cccttagatt ttttcatacg |
| 6361 | atataaactg cattttccat ccaccattaa cctttatcca attctcaata aatgccatta |
| 6421 | atatatttcc atcattttgc tttatttcag ctttccctga aacacaagca atgcctttaa |
| 6481 | actcacgcat tacgacatca tattcatatc tttctatgct aggtaaaatc ctcgctggac |
| 6541 | gcatatcaat ctttcgtaac tgatgttttt tatagataac tttttgacca tttgttgaaa |
| 6601 | taaaccaatc agcttccaag taatctagca tttcagtatt acctgaataa aatgcattat |
| 6661 | accattcttt tcttattaga tttaagtctg atttaaaatt agtcacacta ccttcctcat |
| 6721 | atctaattcc tcctctaatc tatccccaac attccttact gattttatag catcatcaat |
| 6781 | agccacatta tatatgactg ggattactct tttaagaatc tcatcaagta attcctctga |
| 6841 | ttcaaactgt ccaaggcata tatcatgttc atctctgatt ttttgtgaaa taaattcagc |
| 6901 | tagtccttta tatatgtttt tttcaatatc agaaattttc attctttatg aagcctcaca |
| 6961 | aataaattaa taatataaca tttttcctat cataggaaaa ttacaactca accatactgc |
| 7021 | aacctggaat ttcccaagca agcatattta aataagtgca ggataccacc tcgaatcata |
| 7081 | ctctaaggaa caagataatt taccataatc ccttattgta cgcaccgctg aaacgcgttc |
| 7141 | ggcgcgatca cggcagcaga caggtaaaaa tggcaacaaa ccacccgaaa aactgccgcg |
| 7201 | atcgcacccg gtaaatttta accacatgca tagctatgca gccatgtgaa tcacgcagga |
| 7261 | attgccagcc agagacagct gaaacggatt tttttcatgc tttcggaagc gaagaagacg |
| 7321 | gggaccggag cgggaaaaca gaagttcagg ggaatgagcg cgatctggca atagaggcaa |
| 7381 | aacacagcaa caaaagacac accagaatcg cgcccgtatg cgttttaacg cgttcctgtg |
| 7441 | ttttcagcaa ggtctgactg aagcgcgttc ccggtgcccc cgaaccaacc attatcccaa |
| 7501 | aaaatgcacc ggaaacgcgc tgacaacatt ttagcctgtc tctgattacc atcccagcga |
| 7561 | agggccatcc tgtgtccgtt cctgtatttc cagctggcgc tcacgctcca gggataactc |
| 7621 | atgttcgcgt tcctgtatca gccgttcatc ccttatctcc tgttctcgct gtataactgg |
| 7681 | ctcaagcgtt ctgtctgctc gctcaagtgc tgcacctgct gactcaactg catgacccgc |
| 7741 | tcgttcagca tcgcgttgtc ccgttgcgta agcgaaaaca ttttctgcaa ttccacgaag |
| 7801 | gcgctctccc attcgttcag ccgctgcata tagtcctgtt gcagttgctc taaggcgttc |
| 7861 | agcaaatgtc tttccagctc cgtcactctg tgtcactccg tcagttgtac ccagtccttc |
| 7921 | ccctgatagg gaatcaccgt tgccgatttc ccctgcggga taaccagaaa tttccttctc |
| 7981 | ccgtcctgca caaactccac gccccatgtc ttcgcgttca gtttctgcaa tgtttcttcc |
| 8041 | tgtatcctga tttcttccag gttcgcctgt atcatcccgc caagatacca gagcgtcccg |
| 8101 | ccactcacgg taaacaggaa aaggaccatc cccagtaaca tcatgcccgt attccctgcc |
| 8161 | agctttaaca cgtccttcct gtgctgcatc atcgcctctt tcaccccttc ccggtgtttt |
| 8221 | ttcagtgatt cctctgtcga agctgtgaac agggctatag cgtctttgat tttcgtctcg |
| 8281 | tttgatgtca cagccttgct tacagatttt tcgagcttgc tgaactcgtc gttcagcatt |
| 8341 | gtctctgtag attcggctct ctctttcagc tttttctcga actccgcgcc cgtctgtaaa |
| 8401 | agattgctca taaaatgctc ctttcagcct gatattcttc ccaccgttcg ggtctgcaat |
| 8461 | gctgatactg cttcgcatca ccctgaccac ttcaagccct gcctctgtga gcgcctgaat |
| 8521 | cacatcctga cggcctttta tctccccgac atgataaaga gcgtctatcc cgcgtgtgac |
| 8581 | gctctctgca agcgcctgtt tcgtttccgg caggttatca gggagagtca gtatccggcg |
| 8641 | gttctccggg gcgttggggt catgcagccc gtaatggtga ttaaccagcg tctgccaggc |
| 8701 | atcaattctc ggcctgtctg cccggtcgta gtacggctgg aggcgttttc cggtctgtag |
| 8761 | ctccatgttc gggatgacaa aattcagctc aagccgtccc ttgtcctggt gctccaccca |
| 8821 | caggatgctg tactgattct tttcgagacc gggcatcagt acacgctcaa agctcgccat |
| 8881 | caccttttca cgtcctcccg gcggcagctc cttctccgcg aacgacagaa cacctgacgt |
| 8941 | gtatttcttc gcaaatggcg tggcatcgat gagttcccga acttcttccg gattaccctg |
| 9001 | aagcaccgtt gccccttccc ggtttcgctc ccttcccagc aggtaatcaa ccggaccact |
| 9061 | gccaccgcct tttcccctgg catgaaattt aacaatcatt ccgcgctccc tgttccctga |
| 9121 | cgacctgccg taagctgcac aactctctct cgatggccat cagtgcggcc accacctgaa |
| 9181 | cccggtcact ggaagaccac tgcccgctat tcaccttcct cgctgtctga ttcaggttat |
| 9241 | tcccgatggc ggccagctga cgcagtaacg gcggtgccag tgtcggcagc ttcccggagc |
| 9301 | gtgcaaccgg ctcacccagg cagacccgcc gcatccatac cgccagctgt ttaccctcac |
| 9361 | agcgttccag taaccgggca tgttcatcat cagtaacccg tatggtgagc atcctctcgc |
| 9421 | gtttcatcgg tatcattacc ccataaaaca gaaatccccc ttacacggag gcatcagtga |
| 9481 | ctaaacagga aaaaaccgcc cttaacatgg cccgttttat cagaagccag acattaacgc |
| 9541 | tgctggagaa actcaacgaa ctggacgcag atgaacaggc cgatatttgt gaatcacttc |
| 9601 | acgaccacgc cgacgagctt taccgcagct gcctcgcacg cttcggggat gacggtgaaa |
| 9661 | acctctgaca catacagctc ccggagacgg tcacagcttg tctgtgagcg gatgccggga |
| 9721 | gcagacaagc ccgtcagggc gcgtcagggg gtttcggggc gaagccctga accagtcacg |
| 9781 | tagcgatagc ggagtgtata ctggcttaac tatgcggcat cagtgcgaat tgtatggaaa |
| 9841 | gcgcaccatg tccgatgtga aatgccgcac agatgcgtaa ggagaaaatg cacgttccgg |
| 9901 | cgctcttccg cttcctcgct cactgactag ctacactcgg tcgttcggct gcggcgagcg |
| 9961 | gtgtctgctc actcaaaagc ggaggtgcgg ttatccacag aatcagggga taacgccaaa |
| 10021 | agaaacatgt gagcaaaaaa caagaaccag gaaaaggctg cgccgttggc gtttttccat |
| 10081 | aggctccgcc cccctgacga gcatcataaa aatcgacgct caagtcagag gtggcgaaac |
| 10141 | ccgacaggac tataaagata ccaggcgttt ccccctggaa gctccctcgt gcgctctcct |
| 10201 | gttccgaccc tgccgcttac cggatacctg tccgcctttc tcccttcggg aagcgtggcg |
| 10261 | ctttctcata gctcacgctg taggtatctc ggctcggtgt aggtcgttcg ctccaagctg |
| 10321 | ggctgtgtgc acgaaccccc cgttcagccc gaccgctgcg ccttatccgg taactatcgt |
| 10381 | cttgagtcca acccggtaag acacgacaaa gcgccactgg cagcagccac tggtaactgg |
| 10441 | acaaggtgga ttgagatatt aagagttctt gaagtggtgg cctaactgcg gctacactag |
| 10501 | aaggacagta tttggtatct gtgctccacc aagccagtta cccggttaag cagtccccaa |
| 10561 | ctgacttaac cttcgactaa accgcctccc caggcggttt tttcgtttac tggcagcaga |
| 10621 | ttacgcgcag aaaaaaagga tctcaagaag atcctttgat ctttttgaga tccgcccact |
| 10681 | cctggaacgg ggtctgacgc tcagtggaac gaaaactcac gttaagggat tttggtcatg |
| 10741 | agattatcaa aaaggatctt cacctagatc cttttaaatt aaaaatgaag ttttaaatca |
| 10801 | atctaaagta tatatgagta aacttggtct gacagttacc aatgcttaat cagtgaggca |
| 10861 | cctatctcag cgatctgtct atttcgttca tccatagttg cctgactccc cgtcgtgtag |
| 10921 | ataactacga tacgggaggg cttaccatct ggccccagtg ctgcaatgat accgcgagac |
| 10981 | ccacgctcac cggctccaga tttatcagca ataaaccagc cagccggaag ggccgagcgc |
| 11041 | agaagtggtc ctgcaacttt atccgcctcc atccagtcta ttaattgttg ccgggaagct |
| 11101 | agagtaagta gttcgccagt taatagtttg cgcaacgttg ttgccattgc tgcaggcatc |
| 11161 | gtggtgtcac gctcgtcgtt tggtatggct tcattcagct ccggttccca acgatcaagg |
| 11221 | cgagttacat gatcccccat gttgtgcaaa aaagcggtta gctccttcgg tcctccgatc |
| 11281 | gttgtcagaa gtaagttggc agcagtgtta tcactcatgg ttatggcagc actgcataat |
| 11341 | tctcttactg tcatgccatc cgtaagatgc ttttctgtga ctggtgagta ctcaaccaag |
| 11401 | tcattctgag aatagtgtat gcggcgaccg agttgctctt gcccggcgtc aacacgggat |
| 11461 | aataccgcac cacatagcag aactttaaaa gtgctcatca ttggaaaacg ttcttcgggg |
| 11521 | cgaaaactct caaggatctt accgctgttg agatccagtt cgatgtaacc cactcgtgca |
| 11581 | cccaactgat cttcagcatc ttttactttc accagcgttt ctgggtgagc aaaaacagga |
| 11641 | aggcaaaatg ccgcaaaaaa gggaataagg gcgacacgaa aatgttgaat actcatactc |
| 11701 | ttcctttttc aatattattg aagcatttac cagggttatt gtctcatgag cggatacata |
| 11761 | tttgaatgta tttagaaaaa taaacaaata ggggttccgc gcacatttcc ccgaaaagtg |
| 11821 | ccacctgacg tctaagaaac cattattatc atgacattaa cctataaaaa taggcgtatc |
| 11881 | acgaggccct ttcgtcttca agaattttat aaaccgtgga gcgggcaata ctgagctgat |
| 11941 | gagcaatttc cgttgcacca gtgcccttct gatgaagcgt cagcacgacg ttcctgtcca |
| 12001 | cggtacgcct gcggccaaat ttgattcctt tcagctttgc ttcctgtcgg ccctcattcg |
| 12061 | tgcgttctag gatcctccgg cgttcagcct gtgccacagc cgacaggatg gtgaccacca |
| 12121 | tttgccccat atcaccgtcg gtactgatcc cgtcatcaat gaaccggact gccacgccct |
| 12181 | gagcgtcaaa ttcctttatc agttggatca tatcggcagt gtcgcggcca agacggtcga |
| 12241 | gcttcttaac cagaatgaca tcaccttcct ccaccttcat cctcagcaaa tccagccctt |
| 12301 | cccggtctgt tgaactgccg gatgccttat cggtaaatat acggtttgct ttcacacctg |
| 12361 | cgtctttgag tgctctgacc tgaagatcaa gagactgctg actggttgag acccgagcgt |
| 12421 | aaccaaaaag tcgcataaaa atgtacctta aatcgaatat cggacaactc atgtctatta |
| 12481 | ttacaaattt acgatttaat agacatatta atgtaacagt tttacgatgt ccgataattt |
| 12541 | ataacatttc gtacggttgg aaaaatgtta ctaaatgccc gtcaggcagg gaggccgata |
| 12601 | tgcccgttga ctttctgacc actgagcaga ctgaaagcta tggcagattc accggtgaac |
| 12661 | cggatgagct tcagctggca cgatattttc accttgatga agcagacaag gaatttatcg |
| 12721 | gaaaaagcag aggtgatcac aaccgtctgg gcattgccct gcaaattgga tgtgtccgtt |
| 12781 | ttctgggcac cttcctcacc gatatgaatc atattccttc cggcgtccgg cattttaccg |
| 12841 | ccagacagct cgggattcgt gatatcaccg ttcttgcaga atacggtcag aggg |
| // | |

What is claimed is:

1. A method of killing or preventing or decreasing adverse effects of pathogenic Escherichia coli (E. coli) and/or Shigella bacteria, comprising identifying a surface or subject known to or suspected of contamination with said pathogenic E. coli and/or Shigella bacteria; and contacting said pathogenic E. coli and/or Shigella bacteria with a p0.1229_3 containing microcin having one or more of an hp1, abc, cupin, and hp2 or a functional variant thereof.

2. The method of claim 1, wherein the E. coli plasmid 01229_3 containing microcin is a 5 kb (nt 3094 to 7622) region of plasmid 0.1229_3 or a functional variant thereof.

3. The method of claim 1, wherein said 5.2 Kb region encodes SEQ ID NO: 4, 5, 6, and 10.

4. The method of claim 1, wherein said bacteria are selected from the group consisting of: enterohaemorrhagic E. coli (EHEC), enteropathogenic E. coli (EPEC), enterotoxigenic E. coli (ETEC), enteroinvasive E. coli (EIEC), enteroaggregative E. coli (EAEC), diffusively adherent E. coli (DAEC), uropathogenic E. coli (UPEC) and neonatal meningitis E. coli (NMEC).

5. The method of claim 3, wherein said bacteria is enterohaemorrhagic E. coli (EHEC).

6. The method of claim 3, wherein said EHEC is serogroup O157.

7. The method of claim 1, wherein said organism that produces E. coli plasmid 0.1229_3 containing microcin is a naturally occurring non-pathogenic bacteria.

8. The method of claim 7, wherein said naturally occurring bacteria E. coli 0.1229.

9. The method of claim 1, wherein said organism that produces E. coli plasmid 0.1229_3 containing microcin is a genetically modified organism harboring a heterologous nucleic acid which is expressed to produce said E. coli plasmid 0.1229_3 containing microcin having sequences which encode one or more of SEQ ID NOS: 4, 5, 6, and/or 10 or said functional variant thereof.

10. A genetically modified organism selected from the group consisting of virus, bacteria, and yeast, wherein said genetically modified organism harbors a heterologous nucleic acid which is expressed to produce E. coli plasmid 0.1229_3 containing microcin SEQ ID NO:2 nt 3094-7622.

11. The genetically modified organism of claim 10, wherein said organism is bacteria.

12. The genetically modified organism of claim 11, wherein said bacteria is selected from the group consisting of Lactobacillus and Bacteroides.

13. The genetically modified organism of claim 11, wherein said heterologous nucleic acid is present on a plasmid.

14. The genetically modified organism of claim 10, wherein said organism is a virus.

15. The genetically modified organism of claim 14, wherein said virus is an adenovirus or baculovirus.

16. An antimicrobial agent or material comprising the genetically modified organism of claim 10 incorporated into a cleaning agent or material.

17. The antimicrobial agent or material of claim 16, wherein said genetically modified organism is incorporated into a cleaning agent, and said cleaning agent is selected from the group consisting of a soap, gel, spray, and detergent.

18. The antimicrobial agent of material of claim 16, wherein said genetically modified organism is incorporated into a material, and said material is a fabric or sheet.

19. A composition comprising E. coli plasmid 01229_3 containing microcin; and oxidizing agent.

20. A pharmaceutical composition comprising E. coli plasmid 01229 3 containing microcin and a pharmaceutically acceptable carrier.

21. A method for treating or preventing a microbial infection, the method comprising administering to a subject a therapeutically effect amount of the pharmaceutical composition of claim 20.

22. The method of claim 21, wherein the microbial infection is selected from the group consisting of an infection with enteropathogenic E. coli (EPEC), an infection with enterohemorrhagic E. coli (EHEC), and an infection associated with hemolytic-uremic syndrome (HUS).

23. A method for treating or preventing a microbial infection, the method comprising administering to a subject a therapeutically effect amount of the pharmaceutical composition of claim 20.

24. The method of claim 21, wherein the microbial infection is selected from the group consisting of an infection with enteropathogenic E. coli (EPEC), an infection with enterohemorrhagic E. coli (EHEC), and an infection associated with hemolytic-uremic syndrome (HUS).

25. A method for treating or preventing a gastrointestinal disorder, the method comprising administering to a subject a therapeutically effect amount of the pharmaceutical composition of claim 20.

26. A method for treating or preventing a gastrointestinal disorder, the method comprising administering to a subject a therapeutically effect amount of the pharmaceutical composition of claim 20.


SEQUENCE LISTING (81 sequences)

SEQ ID NO: 1 - DNA, 12894 bp, Escherichia coli
aaaatacccgccgtgagcatgcagcgctgatacgtcagcactatcagtatcgtgaatttgcctggccctggacatttcgccttacccgtcttttatatacccggagctggataagcaacgaacgtcctggcctgcttttcgatctggcgacagggtggcttatgcaacatcgtattattctccccggagccactacgctgacccggttgatttcagaggtaagggaaaaggcgacgttgcgcctgtggaacaaactggcactgataccgtcagccgaacagcgttcacagctggagatgctgctggggccaactgattgcagccgcctgtctttactggaatcactgaaaaagggccctgtgaccatcagtggtccggcgtttaatgaagcaattgaacgctggaaaactctgaacgattttggcctgcatgctgaaaacctgagtacactcccggctgtgcgcctgaaaaatctcgcacgttatgctggtatgacttcggtgttcaatattgccaggatgtcaccgcagaaaaggatggcggttctggttgcctttgtccttgcatgggaaacgctggcgctggatgatgcattggacgttctggacgccatgctggccgttatcatccgtgacgccagaaagattgggcagaaaaaacggctccgctcgctgaaggatctggataaatctgcattggcgctcgccagcgcatgttcgtacctgctgaaagaagaaacaccggacgaatcgattcgtgctgaggtgttcagctacatcccaaggcaaaagctggctgaaatcatcacgcttgtccgtgaaattgcccggccctcagacgataattttcatgaagaaatggtggagcagtacgggcgcgttcgtcgtttcctgccccatctgctgaataccgttaaattttcatccgcacctgccggggttaccactctgaatgcctgtgactacctcagccgggagttcagctcacggcggcagttttttgacgacgcaccaacggaaattatcagtcggtcatggaaacggctggtgattaacaaggaaaaacatatcacccgcaggggatacacgctctgctttctcagtaaactgcaggatagtctgaggcggagggatgtctacgttaccggcagtaaccggtggggagatcctcgtgcaagattactacagggtgctgactggcaggcaaaccggattaaggtttatcgttctttggggcacccgacagacccgcaggaagcaataaaatctctgggtcatcagcttgatagtcgttacagacaggttgctgcacgtctttgcgaaaatgaggctgtcgaactcgatgtttctggcccgaagccccggttgacaatttctcccctcgccagtcttgatgagccggacagtctgaaacgactgagcaaaatgatcagtgatctactccctccggtggatttaacggagttgctgctcgaaattaacgcccataccggatttgctgatgagtttttccatgctagtgaagccagtgccagagttgatgatctgcccgtcagcatcagcgccgtgctgatggctgaagcctgcaatatcggtctggaaccactgatcagatcaaatgttcctgcactgacccgacaccggctgaactggacaaaagcgaactatctgcgggctgaaactatcaccagcgctaatgccagactggttgattttcaggcaacgctgccactggcacagatatggggtggaggagaagtggcatctgcagatggaatgcgctttgttacgccagtcagaacaatcaatgccggaccgaaccgcaaatactttggtaataacagagggatcacctggtacaactttgtgtccgatcagtattccggctttcatggcatcgttataccggggacgctgagggactctatctttgtgctggaaggtcttctggaacaggagaccgggctgaatccaaccgaaattatgaccgatacagcaggtgccagcgaacttgtctttggccttttctggctgctgggataccagttttctccacgcctggctgatgccggtgcttcggttttctggcgaatggaccatgatgccgactatggcgtgctgaatgatattgccagagggcaatcagatccccgaaaaatagtccttcagtgggacgaaatgatccggaccgctggctccctgaagctgggcaaagtacaggtttcagtgctggtccgttcattgctgaaaagtgaacgtccttccggactgactcaggcaatcattgaagtggggcgcatcaacaaaacgctgtatctgcttaattatattgatgatgaagattaccgccggcgcattctgacccagcttaatcggggagaaagtcgccatgccgttgccagagccatctgtcacggtcaaaaaggtgagataagaaaacgatataccgacggtcaggaagatcaactgggcacactggggctggtcactaacgccgtcgtgttatggaacactatttatatgcaggcagccctggatcatctccgggcgcagggtgaaacactgaatgatgaagatatcgcacgcctctccccgctttgccacggacatatcaatatgctcggccattattccttcacgctggcagaactggtgaccaaaggacatctgagaccattaaaagaggcgtcagaggcagaaaacgttgcttaacgtgagttttcgttccactgagcgtcagaccccggaacctacaacacatgtgtaaaacgtcaatggagggggctattatcggactgcaacacatttcgtacagcctttacactcggtgaattagcggccctagatgcatccactcattcaaccaactcaattctcttctcttagagtggaaaaattttgttttccttgcagcccctacccgtaaaaactggctgcaaaaaccttgtattactaatggatcctataaccattaaaaaacttgattactatctcatttatagaattaggcatattgacacaagcacaaatttaacaatactataataaatgataactattctcatctacattcaaatatataattgggggtgttatgctacaacataagatgaacagcagttcttatgcaaaagttcataatgttagctcattagaagatatcatgagttatcacaatgatgatgttcttctaaaatttcgtaaagaatggaatgtaacaccagaagaagctgatgatatctttaatgaaacaaagaaattcatctggctagcctcaacatgcctaacggaatgctacaatataaaagttcacgagcaattacaaattatagatgaaatgtggcatacgtttattcaatttacagatgcttataccagcttttgtgaaaaatatcttggtgcttacctccaccattatccaaatacaaatgacatgctaaaaaatgagataaggcatgttaatgagcatggtataacattccaagaatatcgttttaacgaatataaaaatcaaattgaaaaaatcgctttttacctgggtcatgaaactgtcgcaaagtggtatggtgattatgctgtaagatacagcataaagaacattaatactataagaattccaaaagaatccatatccagtgattcttacatcgaaaaagtaaagagtataactcaccttccagccgcagaatttgtaaaaataataatgcgaaaagatgtttggaatgataatggttctgtttgtggttgcagcggtaaaggatgtggcgctgggtgctcatgtaattctagataacataaagcccgtaatatacgggctttaaggattataaattgaattttattgaaaagtacatcatcacatttaataaatgtaatttgttttttattatattattatcaactataagtagttttcttttcgttctattcggatatttaataaaaacagttattgacaataaagattcaataggagaaataaactcattattaatatttatagcatgtttcttatcaattagattccttatgcctgctggatatagcatatcggaatacttgactcaaaaaacaaatatagaattatctgtaaagcttagagagcaagttattgataatatacaaaattctcaccaagagcattttttaaagaaaaataaaggagaactaaataaggttatagaaagcatgctttcttctgcatcatcattattttatacaatatgttctgatgtagtaccattattaatacaaatgattggaataatcataacaatttgtataagtgttaatacattaatagccattgagtttataataataatggcaatctatataatatttgtcataaaaatgacacaacgtagatttccaatgatgaaatcagttgcacttagctccaaattcgcatctggaagaatgtttgatatgatgcacatgtatcctatggataaagctttccataccacggataaaagtcgtaaacgagtaattcaagctgtaaatacacattcagaaaaacaaagaaaagtaaataatgaatttttcctgtttggtatcagctccgcttttctttcagtcatttttagtagtctgattatcctatcagcatactggatgtttcttcatggtagagcaagccgtggtagtattattatgcttgccacttttttattccaagtttttctcccattaaatcgcattggttatctatttagacaaataaaaatggcaagaacagaaattgatttatactgtgccgaaataaacgacataaaaaaaacaaattacacagacaagcaatatcttcatgtaaaagacaatatttgcaatattgaaatatacaacaaatcattcaaccataaattaatattaacaaaaggcgttgttacctttataaccggtgagaatggttcgggaaaaacaactattgcaaaaattctctctggcaatataacaacagaagaaaacacaataaaaatcaatgggatacaacaagaaaaagcaaattctccttttgttaatgtcttatacgttcctcaagatctagatctcatgcctggaactatagaggaaaatatattacattattccggcattaataatttaaatacaattaaaaagctgctacacagatttaagttcgacaaaccactagattatgagatcaaaggttacggacataatttatctggtggacaaaaacaaaaattaggagtatctcttacatctggaaaaaacgtagacttgattattttcgatgaaccgacaaaagggtttgattcacttggtataaaaataatcagtgattatataaaagaggaaaacaaggaaaagcatatcattgttatatctcatgatgaacaacttataaataatatacctaaagcacagattatagatatcacaaaacatgaggtttaaaaatgaataatcttataaaaaaggaaatcatagaaaaatttaagaaatataatttccagaaatatccgtttgtattcacggatgttaattataaaaacttaatcaactggaatgatcttaataaattgcttgaaaaagatatattgcactatcctagagttagaatggcaaatgacaactttcccgaaattagggggtataaaggatttataagatacacatatagccaaacaggcgatagaacaccacatataaatcgccatcagctatataaatgcttacgtgatggcgcaacccttatagtagatcgatgccagtcattctttgaatctgtagatgaaagcagactatggttatctaaagagttagaatgtacatgtagtgctaatctatatgctgcatttacagcaacaccaagctttgggcttcattttgacaatcatgatgtaatagctgttcaaattgagggaataaaaaaatggaaagtttacaaccctacctattcataccctctcgaagatgaaagaagcttcgattatctaccacctaatacctccccagattatgagtttgatataaccccaggtcaggccatatatcttcctgctggatactggcacaatgttacgactcagagcaaacactctcttcatatatcttttacagttataagacctcgtcgattagaactatttaaaacattatttgatgaattaaaaaacaatccatatatgcgggaacctatagagcatggtgattcattatctgataaagaaaaaataaaaactataataactaatgctataaatagttttgatattcagtttgcagaaaacatgcttaaagctcaaagcagaacttatcgctataaaaaaataaaacttgaacatatataaccattaatctaagggggcttccccttagattttttcatacgatataaactgcattttccatccaccattaacctttatccaattctcaataaatgccattaatatatttccatcattttgctttatttcagctttccctgaaacacaagcaatgcctttaaactcacgcattacgacatcatattcatatctttctatgctaggtaaaatcctcgctggacgcatatcaatctttcgtaactgatgttttttatagataactttttgaccatttgttgaaataaaccaatcagcttccaagtaatctagcatttcagtattacctgaataaaatgcattataccattcttttcttattagatttaagtctgatttaaaattagtcacactaccttcctcatatctaattcctcctctaatctatccccaacattccttactgattttatagcatcatcaatagccacattatatatgactgggattactcttttaagaatctcatcaagtaattcctctgattcaaactgtccaaggcatatatcatgttcatctctgattttttgtgaaataaattcagctagtcctttatatatgtttttttcaatatcagaaattttcattctttatgaagcctcacaaataaattaataatataacatttttcctatcataggaaaattacaactcaaccatactgcaacctggaatttcccaagcaagcatatttaaataagtgcaggataccacctcgaatcatactctaaggaacaagataatttaccataatcccttattgtacgcaccgctgaaacgcgttcggcgcgatcacggcagcagacaggtaaaaatggcaacaaaccacccgaaaaactgccgcgatcgcacccggtaaattttaaccacatgcatagctatgcagccatgtgaatcacgcaggaattgccagccagagacagctgaaacggatttttttcatgctttcggaagcgaagaagacggggaccggagcgggaaaacagaagttcaggggaatgagcgcgatctggcaatagaggcaaaacacagcaacaaaagacacaccagaatcgcgcccgtatgcgttttaacgcgttcctgtgttttcagcaaggtctgactgaagcgcgttcccggtgcccccgaaccaaccattatcccaaaaaatgcaccggaaacgcgctgacaacattttagcctgtctctgattaccatcccagcgaagggccatcctgtgtccgttcctgtatttccagctggcgctcacgctccagggataactcatgttcgcgttcctgtatcagccgttcatcccttatctcctgttctcgctgtataactggctcaagcgttctgtctgctcgctcaagtgctgcacctgctgactcaactgcatgacccgctcgttcagcatcgcgttgtcccgttgcgtaagcgaaaacattttctgcaattccacgaaggcgctctcccattcgttcagccgctgcatatagtcctgttgcagttgctctaaggcgttcagcaaatgtctttccagctccgtcactctgtgtcactccgtcagttgtacccagtccttcccctgatagggaatcaccgttgccgatttcccctgcgggataaccagaaatttccttctcccgtcctgcacaaactccacgccccatgtcttcgcgttcagtttctgcaatgtttcttcctgtatcctgatttcttccaggttcgcctgtatcatcccgccaagataccagagcgtcccgccactcacggtaaacaggaaaaggaccatccccagtaacatcatgcccgtattccctgccagctttaacacgtccttcctgtgctgcatcatcgcctctttcaccccttcccggtgttttttcagtgattcctctgtcgaagctgtgaacagggctatagcgtctttgattttcgtctcgtttgatgtcacagccttgcttacagatttttcgagcttgctgaactcgtcgttcagcattgtctctgtagattcggctctctctttcagctttttctcgaactccgcgcccgtctgtaaaagattgctcataaaatgctcctttcagcctgatattcttcccaccgttcgggtctgcaatgctgatactgcttcgcatcaccctgaccacttcaagccctgcctctgtgagcgcctgaatcacatcctgacggccttttatctccccgacatgataaagagcgtctatcccgcgtgtgacgctctctgcaagcgcctgtttcgtttccggcaggttatcagggagagtcagtatccggcggttctccggggcgttggggtcatgcagcccgtaatggtgattaaccagcgtctgccaggcatcaattctcggcctgtctgcccggtcgtagtacggctggaggcgttttccggtctgtagctccatgttcgggatgacaaaattcagctcaagccgtcccttgtcctggtgctccacccacaggatgctgtactgattcttttcgagaccgggcatcagtacacgctcaaagctcgccatcaccttttcacgtcctcccggcggcagctccttctccgcgaacgacagaacacctgacgtgtatttcttcgcaaatggcgtggcatcgatgagttcccgaacttcttccggattaccctgaagcaccgttgccccttcccggtttcgctcccttcccagcaggtaatcaaccggaccactgccaccgccttttcccctggcatgaaatttaacaatcattccgcgctccctgttccctgacgacctgccgtaagctgcacaactctctctcgatggccatcagtgcggccaccacctgaacccggtcactggaagaccactgcccgctattcaccttcctcgctgtctgattcaggttattcccgatggcggccagctgacgcagtaacggcggtgccagtgtcggcagcttcccggagcgtgcaaccggctcacccaggcagacccgccgcatccataccgccagctgtttaccctcacagcgttccagtaaccgggcatgttcatcatcagtaacccgtatggtgagcatcctctcgcgtttcatcggtatcattaccccataaaacagaaatcccccttacacggaggcatcagtgactaaacaggaaaaaaccgcccttaacatggcccgttttatcagaagccagacattaacgctgctggagaaactcaacgaactggacgcagatgaacaggccgatatttgtgaatcacttcacgaccacgccgacgagctttaccgcagctgcctcgcacgcttcggggatgacggtgaaaacctctgacacatacagctcccggagacggtcacagcttgtctgtgagcggatgccgggagcagacaagcccgtcagggcgcgtcagggggtttcggggcgaagccctgaaccagtcacgtagcgatagcggagtgtatactggcttaactatgcggcatcagtgcgaattgtatggaaagcgcaccatgtccgatgtgaaatgccgcacagatgcgtaaggagaaaatgcacgttccggcgctcttccgcttcctcgctcactgactagctacactcggtcgttcggctgcggcgagcggtgtctgctcactcaaaagcggaggtgcggttatccacagaatcaggggataacgccaaaagaaacatgtgagcaaaaaacaagaaccaggaaaaggctgcgccgttggcgtttttccataggctccgcccccctgacgagcatcataaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcggctcggtgtaggtcgttcgctccaagctgggctgtgtgcacgaaccccccgttcagcccgaccgctgcgccttatccggtaactatcgtcttgagtccaacccggtaagacacgacaaagcgccactggcagcagccactggtaactggacaaggtggattgagatattaagagttcttgaagtggtggcctaactgcggctacactagaaggacagtatttggtatctgtgctccaccaagccagttacccggttaagcagtccccaactgacttaaccttcgactaaaccgcctccccaggcggttttttcgtttactggcagcagattacgcgcagaaaaaaaggatctcaagaagatcctttgatctttttgagatccgcccactcctggaacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggcagcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcaccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacgaaaatgttgaatactcatactcttcctttttcaatattattgaagcatttaccagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattttataaaccgtggagcgggcaatactgagctgatgagcaatttccgttgcaccagtgcccttctgatgaagcgtcagcacgacgttcctgtccacggtacgcctgcggccaaatttgattcctttcagctttgcttcctgtcggccctcattcgtgcgttctaggatcctccggcgttcagcctgtgccacagccgacaggatggtgaccaccatttgccccatatcaccgtcggtactgatcccgtcatcaatgaaccggactgccacgccctgagcgtcaaattcctttatcagttggatcatatcggcagtgtcgcggccaagacggtcgagcttcttaaccagaatgacatcaccttcctccaccttcatcctcagcaaatccagcccttcccggtctgttgaactgccggatgccttatcggtaaatatacggtttgctttcacacctgcgtctttgagtgctctgacctgaagatcaagagactgctgactggttgagacccgagcgtaaccaaaaagtcgcataaaaatgtaccttaaatcgaatatcggacaactcatgtctattattacaaatttacgatttaatagacatattaatgtaacagttttacgatgtccgataatttataacatttcgtacggttggaaaaatgttactaaatgcccgtcaggcagggaggccgatatgcccgttgactttctgaccactgagcagactgaaagctatggcagattcaccggtgaaccggatgagcttcagctggcacgatattttcaccttgatgaagcagacaaggaatttatcggaaaaagcagaggtgatcacaaccgtctgggcattgccctgcaaattggatgtgtccgttttctgggcaccttcctcaccgatatgaatcatattccttccggcgtccggcattttaccgccagacagctcgggattcgtgatatcaccgttcttgcagaatacggtcagaggg

SEQ ID NO: 2 - DNA, 12894 bp, Escherichia coli; identical to SEQ ID NO: 1

SEQ ID NO: 3 - protein, 1001 aa, Escherichia coli
MPVDFLTTEQTESYGRFTGEPDELQLARYFHLDEADKEFIGKSRGDHNRLGIALQIGCVRFLGTFLTDMNHIPSGVRHFTARQLGIRDITVLAEYGQRENTRREHAALIRQHYQYREFAWPWTFRLTRLLYTRSWISNERPGLLFDLATGWLMQHRIILPGATTLTRLISEVREKATLRLWNKLALIPSAEQRSQLEMLLGPTDCSRLSLLESLKKGPVTISGPAFNEAIERWKTLNDFGLHAENLSTLPAVRLKNLARYAGMTSVFNIARMSPQKRMAVLVAFVLAWETLALDDALDVLDAMLAVIIRDARKIGQKKRLRSLKDLDKSALALASACSYLLKEETPDESIRAEVFSYIPRQKLAEIITLVREIARPSDDNFHEEMVEQYGRVRRFLPHLLNTVKFSSAPAGVTTLNACDYLSREFSSRRQFFDDAPTEIISRSWKRLVINKEKHITRRGYTLCFLSKLQDSLRRRDVYVTGSNRWGDPRARLLQGADWQANRIKVYRSLGHPTDPQEAIKSLGHQLDSRYRQVAARLCENEAVELDVSGPKPRLTISPLASLDEPDSLKRLSKMISDLLPPVDLTELLLEINAHTGFADEFFHASEASARVDDLPVSISAVLMAEACNIGLEPLIRSNVPALTRHRLNWTKANYLRAETITSANARLVDFQATLPLAQIWGGGEVASADGMRFVTPVRTINAGPNRKYFGNNRGITWYNFVSDQYSGFHGIVIPGTLRDSIFVLEGLLEQETGLNPTEIMTDTAGASELVFGLFWLLGYQFSPRLADAGASVFWRMDHDADYGVLNDIARGQSDPRKIVLQWDEMIRTAGSLKLGKVQVSVLVRSLLKSERPSGLTQAIIEVGRINKTLYLLNYIDDEDYRRRILTQLNRGESRHAVARAICHGQKGEIRKRYTDGQEDQLGTLGLVTNAVVLWNTIYMQAALDHLRAQGETLNDEDIARLSPLCHGHINMLGHYSFTLAELVTKGHLRPLKEASEAENVA

SEQ ID NO: 4 - protein, 232 aa, Escherichia coli
MLQHKMNSSSYAKVHNVSSLEDIMSYHNDDVLLKFRKEWNVTPEEADDIFNETKKFIWLASTCLTECYNIKVHEQLQIIDEMWHTFIQFTDAYTSFCEKYLGAYLHHYPNTNDMLKNEIRHVNEHGITFQEYRFNEYKNQIEKIAFYLGHETVAKWYGDYAVRYSIKNINTIRIPKESISSDSYIEKVKSITHLPAAEFVKIIMRKDVWNDNGSVCGCSGKGCGAGCSCNSR

SEQ ID NO: 5 - protein, 530 aa, Escherichia coli
MNFIEKYIITFNKCNLFFIILLSTISSFLFVLFGYLIKTVIDNKDSIGEINSLLIFIACFLSIRFLMPAGYSISEYLTQKTNIELSVKLREQVIDNIQNSHQEHFLKKNKGELNKVIESMLSSASSLFYTICSDVVPLLIQMIGIIITICISVNTLIAIEFIIIMAIYIIFVIKMTQRRFPMMKSVALSSKFASGRMFDMMHMYPMDKAFHTTDKSRKRVIQAVNTHSEKQRKVNNEFFLFGISSAFLSVIFSSLIILSAYWMFLHGRASRGSIIMLATFLFQVFLPLNRIGYLFRQIKMARTEIDLYCAEINDIKKTNYTDKQYLHVKDNICNIEIYNKSFNHKLILTKGVVTFITGENGSGKTTIAKILSGNITTEENTIKINGIQQEKANSPFVNVLYVPQDLDLMPGTIEENILHYSGINNLNTIKKLLHRFKFDKPLDYEIKGYGHNLSGGQKQKLGVSLTSGKNVDLIIFDEPTKGFDSLGIKIISDYIKEENKEKHIIVISHDEQLINNIPKAQIIDITKHEV

SEQ ID NO: 6 - protein, 297 aa, Escherichia coli
MNNLIKKEIIEKFKKYNFQKYPFVFTDVNYKNLINWNDLNKLLEKDILHYPRVRMANDNFPEIRGYKGFIRYTYSQTGDRTPHINRHQLYKCLRDGATLIVDRCQSFFESVDESRLWLSKELECTCSANLYAAFTATPSFGLHFDNHDVIAVQIEGIKKWKVYNPTYSYPLEDERSFDYLPPNTSPDYEFDITPGQAIYLPAGYWHNVTTQSKHSLHISFTVIRPRRLELFKTLFDELKNNPYMREPIEHGDSLSDKEKIKTIITNAINSFDIQFAENMLKAQSRTYRYKKIKLEHI

SEQ ID NO: 7 - protein, 120 aa, Escherichia coli
MTNFKSDLNLIRKEWYNAFYSGNTEMLDYLEADWFISTNGQKVIYKKHQLRKIDMRPARILPSIERYEYDVVMREFKGIACVSGKAEIKQNDGNILMAFIENWIKVNGGWKMQFISYEKI

SEQ ID NO: 8 - protein, 79 aa, Escherichia coli
MKISDIEKNIYKGLAEFISQKIRDEHDICLGQFESEELLDEILKRVIPVIYNVAIDDAIKSVRNVGDRLEEELDMRKVV

SEQ ID NO: 9 - protein, 59 aa, Escherichia coli
MWLKFTGCDRGSFSGGLLPFLPVCCRDRAERVSAVRTIRDYGKLSCSLEYDSRWYPALI

SEQ ID NO: 10 - protein, 65 aa, Escherichia coli
MSYPWSVSASWKYRNGHRMALRWDGNQRQAKMLSARFRCIFWDNGWFGGTGNALQSDLAENTGTR

SEQ ID NO: 11 - protein, 517 aa, Escherichia coli
MIVKFHARGKGGGSGPVDYLLGRERNREGATVLQGNPEEVRELIDATPFAKKYTSGVLSFAEKELPPGGREKVMASFERVLMPGLEKNQYSILWVEHQDKGRLELNFVIPNMELQTGKRLQPYYDRADRPRIDAWQTLVNHHYGLHDPNAPENRRILTLPDNLPETKQALAESVTRGIDALYHVGEIKGRQDVIQALTEAGLEVVRVMRSSISIADPNGGKNIRLKGAFYEQSFTDGRGVREKAERESRIYRDNAERRVQQARKICKQGCDIKRDENQRRYSPVHSFDRGITEKTPGRGERGDDAAQEGRVKAGREYGHDVTGDGPFPVYREWRDALVSWRDDTGEPGRNQDTGRNIAETEREDMGRGVCAGREKEISGYPAGEIGNGDSLSGEGLGTTDGVTQSDGAGKTFAERLRATATGLYAAAERMGERLRGIAENVFAYATGQRDAERAGHAVESAGAALERADRTLEPVIQREQEIRDERLIQEREHELSLERERQLEIQERTQDGPSLGW

SEQ ID NO: 12 - protein, 107 aa, Escherichia coli
MLTIRVTDDEHARLLERCEGKQLAVWMRRVCLGEPVARSGKLPTLAPPLLRQLAAIGNNLNQTARKVNSGQWSSSDRVQVVAALMAIERELCSLRQVVREQGARNDC

SEQ ID NO: 13 - protein, 63 aa, Escherichia coli
MTKQEKTALNMARFIRSQTLTLLEKLNELDADEQADICESLHDHADELYRSCLARFGDDGENL

SEQ ID NO: 14 - protein, 79 aa, Escherichia coli
MFIVLSGFATSDLSVDFYDARQGGGAYGKTPTAQPFPGSCFLLTCFFWRYPLILWITAPPLLSEQTPLAAAERPSVASQ

SEQ ID NO: 15 - protein, 286 aa, Escherichia coli [sequence omitted]

SEQ ID NO: 16 - protein, 185 aa, Escherichia coli
MRLFGYARVSTSQQSLDLQVRALKDAGVKANRIFTDKASGSSTDREGLDLLRMKVEEGDVILVKKLDRLGRDTADMIQLIKEFDAQGVAVRFIDDGISTDGDMGQMVVTILSAVAQAERRRILERTNEGRQEAKLKGIKFGRRRTVDRNVVLTLHQKGTGATEIAHQLSIARSTVYKILEDERAS

SEQ ID NO: 17 - DNA, 70 bp, Escherichia coli
atactattcagatgtcataagcattaatttcccttaaaaaaggagtccttgtgtaggctggagctgcttc

SEQ ID NO: 18 - DNA, 70 bp, Escherichia coli
ttttttaatatcagggagcaccatgctccctgaacggttaattcaacgtacatatgaatatcctccttag

SEQ ID NO: 19 - DNA, 20 bp, Escherichia coli
ggggcttaaaggggtagtgt

SEQ ID NO: 20 - DNA, 21 bp, Escherichia coli
aagcgattcgtccagtagttt

SEQ ID NO: 21 - DNA, 70 bp, Escherichia coli
gtccggttctgaggaggggcccgtccgggcaaccggcgggtctactcacccatatgaatatcctccttag

SEQ ID NO: 22 - DNA, 20 bp, Escherichia coli
cctaacaacgccacgacttt

SEQ ID NO: 23 - DNA, 70 bp, Escherichia coli
acacatttcgtacagcctttacactcggtgaattagcggccctagatgcagtgtaggctggagctgcttc

SEQ ID NO: 24 - DNA, 70 bp, Escherichia coli
ttaaacctcatgttttgtgatatctataatctgtgctttaggtatattatcatatgaatatcctccttag

SEQ ID NO: 25 - DNA, 20 bp, Escherichia coli
gaagatatcgcacgcctctc

SEQ ID NO: 26 - DNA, 20 bp, Escherichia coli
cgcctgtttggctatatgtg

SEQ ID NO: 27 - DNA, 70 bp, Escherichia coli
aatatacctaaagcacagattatagatatcacaaaacatgaggtttaaaagtgtaggctggagctgcttc

SEQ ID NO: 28 - DNA, 70 bp, Escherichia coli
tggagtttgtgcaggacgggagaaggaaatttctggttatcccgcaggggcatatgaatatcctccttag

SEQ ID NO: 29 - DNA, 20 bp, Escherichia coli
ttcgatgaaccgacaaaagg

SEQ ID NO: 30 - DNA, 19 bp, Escherichia coli
gggtgaaagaggcgatgat

SEQ ID NO: 31 - DNA, 70 bp, Escherichia coli
tttaccgcagctgcctcgcacgcttcggggatgacggtgaaaacctctgagtgtaggctggagctgcttc

SEQ ID NO: 32 - DNA, 70 bp, Escherichia coli
agcagacaccgctcgccgcagccgaacgaccgagtgtagctagtcagtgacatatgaatatcctccttag

SEQ ID NO: 33 - DNA, 20 bp, Escherichia coli
cacggaggcatcagtgacta

SEQ ID NO: 34 - DNA, 20 bp, Escherichia coli
cagccttttcctggttcttg

SEQ ID NO: 35 - DNA, 70 bp, Escherichia coli
aattctagataacataaagcccgtaatatacgggctttaaggattataaagtgtaggctggagctgcttc

SEQ ID NO: 36 - DNA, 70 bp, Escherichia coli
atatttcttaaatttttctatgatttccttttttataagattattcatttcatatgaatatcctccttag

SEQ ID NO: 37 - DNA, 21 bp, Escherichia coli
gcgaaaagatgtttggaatga

SEQ ID NO: 38 - DNA, 20 bp, Escherichia coli
tcgggaaagttgtcatttgc

SEQ ID NO: 39 - DNA, 70 bp, Escherichia coli
ataaatgataactattctcatctacattcaaatatataattgggggtgttgtgtaggctggagctgcttc

SEQ ID NO: 40 - DNA, 70 bp, Escherichia coli
aataaaattcaatttataatccttaaagcccgtatattacgggctttatgcatatgaatatcctccttag

SEQ ID NO: 41 - DNA, 20 bp, Escherichia coli
actggctgcaaaaaccttgt

SEQ ID NO: 42 - DNA, 26 bp, Escherichia coli
tttctcctattgaatctttattgtca

SEQ ID NO: 43 - DNA, 70 bp, Escherichia coli; identical to SEQ ID NO: 27

SEQ ID NO: 44 - DNA, 70 bp, Escherichia coli
agtttatatcgtatgaaaaaatctaaggggaagcccccttagattaatggcatatgaatatcctccttag

SEQ ID NO: 45 - DNA, 23 bp, Escherichia coli
aaagaggaaaacaaggaaaagca

SEQ ID NO: 46 - DNA, 20 bp, Escherichia coli
gcattgcttgtgtttcaggg

SEQ ID NO: 47 - DNA, 69 bp, Escherichia coli
aaaaataaaacttgaacatatataaccattaatctaagggggcttccccgtgtaggctggagctgcttc

SEQ ID NO: 48 - DNA, 70 bp, Escherichia coli
aggaatgttggggatagattagaggaggaattagatatgaggaaggtagtcatatgaatatcctccttag

SEQ ID NO: 49 - DNA, 70 bp, Escherichia coli
tgcattataccattcttttcttattagatttaagtctgatttaaaattaggtgtaggctggagctgcttc

SEQ ID NO: 50 - DNA, 70 bp, Escherichia coli
tgataggaaaaatgttatattattaatttatttgtgaggcttcataaagacatatgaatatcctccttag

SEQ ID NO: 51 - DNA, 21 bp, Escherichia coli
ggcacaatgttacgactcaga

SEQ ID NO: 52 - DNA, 20 bp, Escherichia coli
gtttcagcggtgcgtacaat

SEQ ID NO: 53 - DNA, 70 bp, Escherichia coli
aaattacaactcaaccatactgcaacctggaatttcccaagcaagcatatgtgtaggctggagctgcttc

SEQ ID NO: 54 - DNA, 70 bp, Escherichia coli
tgtctctggctggcaattcctgcgtgattcacatggctgcatagctatgccatatgaatatcctccttag

SEQ ID NO: 55 - DNA, 23 bp, Escherichia coli
tcctctgattcaaactgtccaag

SEQ ID NO: 56 - DNA, 20 bp, Escherichia coli
tgttgctgtgttttgcctct

SEQ ID NO: 57 - DNA, 70 bp, Escherichia coli
aggcaaaacacagcaacaaaagacacaccagaatcgcgcccgtatgcgttgtgtaggctggagctgcttc

SEQ ID NO: 58 - DNA, 70 bp, Escherichia coli
acagcgagaacaggagataagggatgaacggctgatacaggaacgcgaaccatatgaatatcctccttag

SEQ ID NO: 59 - DNA, 20 bp, Escherichia coli
gaattgccagccagagacag

SEQ ID NO: 60 - DNA, 20 bp, Escherichia coli
ggtcatgcagttgagtcagc

SEQ ID NO: 61 - DNA, 70 bp, Escherichia coli
tgcatttaaaatcgagacctggtttttctactgaaatgattatgacttcagtgtaggctggagctgcttc

SEQ ID NO: 62 - DNA, 70 bp, Escherichia coli
ctgttgagtaatagtcaaaagcctccggtcggaggcttttgactttctgccatatgaatatcctccttag

SEQ ID NO: 63 - DNA, 20 bp, Escherichia coli
aacatacaacacgggcacaa

SEQ ID NO: 64 - DNA, 20 bp, Escherichia coli
gacgacatcggtcagcatta

SEQ ID NO: 65 - DNA, 70 bp, Escherichia coli
aattttacagtttgatcgcgctaaatactgcttcaccacaaggaatgcaagtgtaggctggagctgcttc

SEQ ID NO: 66 - DNA, 70 bp, Escherichia coli
atctttacgttgccttacgttcagacggggccgaagccccgtcgtcgtcacatatgaatatcctccttag

SEQ ID NO: 67 - DNA, 20 bp, Escherichia coli
ccaaatgtaacgggcaggtt

SEQ ID NO: 68 - DNA, 20 bp, Escherichia coli
gcgtggcgtatggattttgt

SEQ ID NO: 69 - DNA, 60 bp, Escherichia coli
gctagcgaattcgagctcggtacccgggggaatccgcaataattttacagtttgatcgcg

SEQ ID NO: 70 - DNA, 58 bp, Escherichia coli
gcttgcatgcctgcaggtcgactctagaggataacccgtatctttacgttgccttacg

SEQ ID NO: 71 - DNA, 58 bp, Escherichia coli
cgtaaggcaacgtaaagatacgggttatcctctagagtcgacctgcaggcatgcaagc

SEQ ID NO: 72 - DNA, 60 bp, Escherichia coli
cgcgatcaaactgtaaaattattgcggattcccccgggtaccgagctcgaattcgctagc

SEQ ID NO: 73 - DNA, 19 bp, Escherichia coli
ctgtttctccatacccgtt

SEQ ID NO: 74 - DNA, 17 bp, Escherichia coli
ctcatccgccaaaacag

SEQ ID NO: 75 - DNA, 43 bp, Escherichia coli
gtatatatgagtaaacttggtctgacagcattaaaagaggcgt

SEQ ID NO: 76 - DNA, 15 bp, Escherichia coli
cagaggcagaaaacg

SEQ ID NO: 77 - DNA, 56 bp, Escherichia coli
gcggcattttgccttcctgtttttgcgaaatcggcaacggtgattccctatcaggg

SEQ ID NO: 78 - DNA, 56 bp, Escherichia coli
ccctgatagggaatcaccgttgccgatttcgcaaaaacaggaaggcaaaatgccgc

SEQ ID NO: 79 - DNA, 58 bp, Escherichia coli
cgttttctgcctctgacgcctcttttaatgctgtcagaccaagtttactcatatatac

SEQ ID NO: 80 - DNA, 20 bp, Escherichia coli
tttgcaagcagcagattacg

SEQ ID NO: 81 - DNA, 20 bp, Escherichia coli
gcctcgtgatacgcctattt